SEQUENCE LISTING (549 sequences)

SEQ ID NO: 1 - DNA, 3182 bp, synthetic construct
  source 1..3182: mol_type = other DNA
aattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtgg

SEQ ID NO: 2 - DNA, 3221 bp, synthetic construct
  source 1..3221: mol_type = other DNA
ttccactgccttccaccaagctctgcaggatcccagagtcaggggtctgtattttcctgctggtggctccagttcaggaacagtaaaccctgctccgaatattgcctctcacatctcgtcaatctccgcgaggactggggaccctgtgacgaacatggagaacatcacatcaggattcctaggacccctgctcgtgttacaggcggggtttttcttgttgacaagaatcctcacaataccgcagagtctagactcgtggtggacttctctcaattttctagggggatcacccgtgtgtcttggccaaaattcgcagtccccaacctccaatcactcaccaacctcctgtcctccaatttgtcctggttatcgctggatgtgtctgcggcgttttatcatattcctcttcatcctgctgctatgcctcatcttcttattggttcttctggattatcaaggtatgttgcccgtttgtcctctaattccaggatcaacaacaaccagtacgggaccatgcaaaacctgcacgactcctgctcaaggcaactctatgtttccctcatgttgctgtacaaaacctacggatggaaattgcacctgtattcccatcccatcgtcctgggctttcgcaaaatacctatgggagtgggcctcagtccgtttctcttggctcagtttactagtgccatttgttcagtggttcgtagggctttcccccactgtttggctttcagctatatggatgatgtggtattgggggccaagtctgtacagcatcgtgagtccctttataccgctgttaccaattttcttttgtctctgggtatacatttaaaccctaacaaaacaaaaagatggggttattccctaaacttcatgggttacataattggaagttggggaacattgccacaggatcatattgtacaaaagatcaaacactgttttagaaaacttcctgttaacaggcctattgattggaaagtatgtcaaagaattgtgggtcttttgggctttgctgctccatttacacaatgtggatatcctgccttaatgcctttgtatgcatgtatacaagctaaacaggctttcactttctcgccaacttacaaggcctttctaagtaaacagtacatgaacctttaccccgttgctcggcaacggcctggtctgtgccaagtgtttgctgacgcaacccccactggctggggcttggccataggccatcagcgcatgcgtggaacctttgtggctcctctgccgatccatactgcggaactcctagccgcttgttttgctcgcagccggtctggagcaaagctcatcggaactgacaattctgtcgtcctctcgcggaaatatacatcgtttccatggctgctaggctgtactgccaactggatccttcgcgggacgtcctttgtttacgtcccgtcggcgctgaatcccgcggacgacccctctcggggccgcttgggactctctcgtccccttctccgtctgccgttccagccgaccacggggcgcacctctctttacgcggtctccccgtctgtgccttctcatctgccggtccgtgtgcacttcgcttcacctctgcacgttgcatggagaccaccgtgaacgcccatcagatcctgcccaaggtcttacataagaggactcttggactcccagcaatgtcaacgaccgaccttgaggcctacttcaaagactgtgtgtttaaggactgggaggagctgggggaggagattaggttaaaggtctttgtattaggaggctgtaggcataaattggtctgcgcaccagcaccatgcaactttttcacctctgcctaatcatctcttgtacatgtcccactgttcaagcctccaagctgtgccttgggtggctttggggcatggacattgacccttataaagaatttggagctactgtggagttactctcgtttttgccttctgacttctttccttccgtcagagatctcctagacaccgcctcagctctgtatcgggaagccttagagtctcctgagcattgctcacctcaccatactgcactcaggcaagccattctctgctggggggaattgatgactctagctacctgggtgggtaataatttggaagatccagcatccagggatctagtagtcaattatgttaatactaacatgggtttaaagatcaggcaactattgtggtttcatatatcttgccttacttttggaagagagactgtacttgaatatttggtctctttcggagtgtggattcgcactcctccagcctatagaccaccaaatgcccctatcttatcaacacttccggaaactactgttgttagacgacgggaccgaggcaggtcccctagaagaagaactccctcgcctcgcagacgcagatctcaatcgccgcgtcgcagaagatctcaatctcgggaatctcaatgttagtattccttggactcataaggtgggaaactttactgggctttattcctctacagtacctatctttaatcctgaatggcaaactccttcctttcctaagattcatttacaagaggacattattaataggtgtcaacaatttgtgggccctctcactgtaaatgaaaagagaagattgaaattaattatgcctgctagattctatcctacccacactaaatatttgcccttagacaaaggaattaaaccttattatccagatcaggtagttaatcattacttccaaaccagacattatttacatactctttggaaggctggtattctatataagagggaaaccacacgtagcgcatcattttgcgggtcaccatattcttgggaacaagagctacagcatgggaggttggtcatcaaaacctcgcaaaggcatggggacgaatctttctgttcccaaccctctgggattctttcccgatcatcagttggaccctgcattcggagccaactcaaacaatccagattgggacttcaaccccatcaaggaccactggccagcagccaaccaggtaggagtgggagcattcgggccagggttcacccctccacacggcggtgttttggggtggagccctcaggctcagggcatattgaccacagtgtcaacaattcctcctcctgcctccaccaatcggcagtcaggaaggcagcctactcccatctctccacctctaagagacagtcatcctcaggccatgcagtggaa

SEQ ID NO: 3 - DNA, 3215 bp, synthetic construct
  source 1..3215: mol_type = other DNA
ctccaccactttccaccaaactcttcaagatcccagagtcagggccctgtactttcctgctggtggctccagttcaggaacagtgagccctgctcagaatactgtctctgccatatcgtcaatcttatcgaagactggggaccctgtaccgaacatggagaacatcgcatcaggactcctaggacccctgctcgtgttacaggcggggtttttcttgttgacaaaaatcctcacaataccacagagtctagactcgtggtggacttctctcaattttctagggggaacacccgtgtgtcttggccaaaattcgcagtcccaaatctccagtcactcaccaacctgttgtcctccaatttgtcctggttatcgctggatgtgtctgcggcgttttatcatcttcctctgcatcctgctgctatgcctcatcttcttgttggttcttctggactatcaaggtatgttgcccgtttgtcctctaattccaggatcatcaacaaccagcaccggaccatgcaaaacctgcacaactcctgctcaaggaacctctatgtttccctcatgttgctgtacaaaacctacggacggaaactgcacctgtattcccatcccatcatcttgggctttcgcaaaatacctatgggagtgggcctcagtccgtttctcttggctcagtttactagtgccatttgttcagtggttcgtagggctttcccccactgtctggctttcagttatatggatgatgtggttttgggggccaagtctgtacaacatcttgagtccctttatgccgctgttaccaattttcttttgtctttgggtatacatttaaaccctcacaaaacaaaaagatggggatattcccttaacttcatgggatatgtaattgggagttggggcacattgccacaggaacatattgtacaaaaaatcaaaatgtgttttaggaaacttcctgtaaacaggcctattgattggaaagtatgtcaacgaattgtgggtcttttggggtttgccgcccctttcacgcaatgtggatatcctgctttaatgcctttatatgcatgtatacaagcaaaacaggcttttactttctcgccaacttacaaggcctttctaagtaaacagtatctgaacctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggggcaaaactcatcgggactgacaattctgtcgtgctctcccgcaagtatacatcatttccatggctgctaggctgtgctgccaactggatcctgcgcgggacgtcctttgtttacgtcccgtcggcgctgaatcccgcggacgacccctcccggggccgcttggggctctaccgcccgcttctccgcctgttgtaccgaccgaccacggggcgcacctctctttacgcggactccccgtctgtgccttctcatctgccggaccgtgtgcacttcgcttcacctctgcacgtcgcatggagaccaccgtgaacgcccaccggaacctgcccaaggtcttgcataagaggactcttggactttcagcaatgtcaacgaccgaccttgaggcatacttcaaagactgtgtgtttactgagtgggaggagttgggggaggagattaggttaaaggtctttgtactaggaggctgtaggcataaattggtgtgttcaccagcaccatgcaactttttcacctctgcctaatcatctcatgttcatgtcctactgttcaagcctccaagctgtgccttgggtggctttggggcatggacattgacccgtataaagaatttggagcttctgtggagttactctcttttttgccttctgacttctttccttctattcgagatctcctcgacaccgcctctgctctgtatcgggaggccttagagtctccggaacattgttcacctcaccatacggcactcaggcaagctattctgtgttggggtgagttgatgaatctagccacctgggtgggaagtaatttggaagatccagcatccagggaattagtagtcagctatgtcaacgttaatatgggcctaaaaatcagacaactattgtggtttcacatttcctgtcttacttttgggagagaaactgttcttgaatatttggtgtcttttggagtgtggattcgcactcctcctgcatatagaccaccaaatgcccctatcttatcaacacttccggaaactactgttgttagacgaagaggcaggtcccctagaagaagaactccctcgcctcgcagacgaaggtctcaatcgccgcgtcgcagaagatctcaatctcgggaatctcaatgttagtattccttggacacataaggtgggaaactttacggggctttattcttctacggtaccttgctttaatcctaaatggcaaactccttcttttcctgacattcatttgcaggaggacattgttgatagatgtaagcaatttgtggggccccttacagtaaatgaaaacaggagactaaaattaattatgcctgctaggttttatcccaatgttactaaatatttgcccttagataaagggatcaaaccgtattatccagagcatgtagttaatcattacttccagacgagacattatttacacactctttggaaggcgggtatcttatataaaagagagtccacacgtagcgcctcattttgcgggtcaccatattcttgggaacaagatctacagcatgggaggttggtcttccaaacctcgaaaaggcatggggacaaatctttctgtccccaatcccctgggattcttccccgatcatcagttggaccctgcattcaaagccaactcagaaaatccagattgggacctcaacccgcacaaggacaactggccggacgccaacaaggtgggagtgggagcattcgggccagggttcacccctccccatgggggactgttggggtggagccctcaggctcagggcatactcacaactgtgccagcagctcctcctcctgcctccaccaatcggcagtcaggaaggcagcctactcccttatctccacctctaagggacactcatcctcaggccatgcagtggaa

SEQ ID NO: 4 - DNA, 3223 bp, synthetic construct
  source 1..3223: mol_type = other DNA
ctccacaacattccaccaagctctgctagatcccagagtgaggggcctatactttcctgctggtggctccagttccggaacagtaaaccctgttccgactactgcctcacccatatcgtcaatcttctcgaggactggggaccctgcaccgaacatggagaacacaacatcaggattcctaggacccctgctcgtgttacaggcggggtttttcttgttgacaagaatcctcacaataccacagagtctagactcgtggtggacttctctcaattttctagggggagcacccacgtgtcctggccaaaattcgcagtccccaacctccaatcactcaccaacctcttgtcctccaatttgtcctggctatcgctggatgtgtctgcggcgttttatcatattcctcttcatcctgctgctatgcctcatcttcttgttggttcttctggactaccaaggtatgttgcccgtttgtcctctacttccaggaacatcaactaccagcacgggaccatgcaagacctgcacgattcctgctcaaggaacctctatgtttccctcttgttgctgtacaaaaccttcggacggaaactgcacttgtattcccatcccatcatcctgggctttcgcaagattcctatgggagtgggcctcagtccgtttctcctggctcagtttactagtgccatttgttcagtggttcgtagggctttcccccactgtttggctttcagttatatggatgatgtggtattgggggccaagtctgtacaacatcttgagtccctttttacctctattaccaattttcttttgtctttgggtatacatttgaaccctaataaaaccaaacgttggggctactcccttaacttcatgggatatgtaattggaagttggggtactttaccacaggaacatattgtactaaaaatcaagcaatgttttcgaaaactgcctgtaaatagacctattgattggaaagtatgtcaaagaattgtgggtcttttgggctttgctgccccttttacacaatgtggctatcctgccttaatgcctttatatgcatgtatacaatctaagcaggctttcactttctcgccaacttacaaggcctttctgtgtaaacaatatctgaacctttaccccgttgcccggcaacggtcaggtctctgccaagtgtttgctgacgcaacccccactggatggggcttggccataggccatcggcgcatgcgtggaacctttgtggctcctctgccgatccatactgcggaactcctagcagcttgttttgctcgcagccggtctggagcgaaacttatcggaaccgacaactctgttgtcctctctcggaaatacacctcctttccatggctgctagggtgtgctgccaactggatcctgcgcgggacgtcctttgtctacgtcccgtcggcgctgaatcccgcggacgacccgtctcggggccgtttgggactctaccgtccccttcttcatctgccgttccggccgaccacggggcgcacctctctttacgcggtctccccgtctgtgccttctcatctgccggaccgtgtgcacttcgcttcacctctgcacgtcgcatggagaccaccgtgaacgcccaccaggtctccaaggtttgcccaaggtcttacataagaggactcttggactctcagcaatgtcaacgaccgaccttgaggcatacttcaaagactgtttgtttaaagactgggaggagttgggggaggagattaggttaatgatctttgtactaggaggctgtaggcataaattggtctgttcaccagcaccatgcaactttttcacctctgcctaatcatctcatgttcatgtcctactgttcaagcctccaagctgtgccttgggtggctttggggcatggacattgacccgtataaagaatttggagcttctgtggagttactctcttttttgccttctgacttctttccttctattcgagatctcctcgacaccgcctctgctctgtatcgggaggccttagagtctccggaacattgttcacctcaccatacagcactcaggcaagctattctgtgttggggtgagttgatgaatctggccacctgggtgggaagtaatttggaagacccagcatccagggaattagtagtcagctatgtcaatgttaatatgggcctaaaaatcagacaactattgtggtttcacatttcctgtcttacttttggaagagaaactgttcttgagtatttggtgtcttttggagtgtggattcgcactcctcccgcttacagaccaccaaatgcccctatcttatcaacacttccggaaactactgttgttagacgacgaggcaggtcccctagaagaagaactccctcgcctcgcagacgaaggtctcaatcgccgcgtcgcagaagatctcaatctcgggaatctcaatgttagtatcccttggactcataaggtgggaaactttactgggctttattcttctactgtacctgtctttaatcctgagtggcaaactccctcctttcctcacattcatttacaggaggacattattaatagatgtcaacaatatgtgggccctcttacagttaatgaaaaaaggagattaaaattaattatgcctgctaggttctatcctaaccttaccaaatatttgcccttggacaaaggcattaaaccttattatcctgaacatgcagttaatcattacttcaaaactaggcattatttacatactctgtggaaggctggcattctatataagagagaaactacacgcagcgcctcattttgtgggtcaccatattcttgggaacaagagctacagcatgggaggttggtcttccaaacctcgacaaggcatggggacgaatctttctgttcccaatcctctgggattctttcccgatcaccagttggaccctgcgttcggagccaactcaaacaatccagattgggacttcaaccccaacaaggatcactggccagaggcaaatcaggtaggagcgggagcattcgggccagggttcaccccaccacacggcggtcttttggggtggagccctcaggctcagggcatattgacaacagtgccagcagcacctcctcctgcctccaccaatcggcagtcaggaagacagcctactcccatctctccacctctaagagacagtcatcctcaggccatgcagtggaa

SEQ ID NO: 5 - DNA, 3219 bp, synthetic construct
  source 1..3219: mol_type = other DNA
ctccaccacattccaccaagctctgctagatcccagagtgaggggcctatactttcctgctggtggctccagttccggaacagtaaaccctgttccgactactgcctctcccatatcgtcaatcttctcgaggactggggaccctgcaccgaacatggagaacaccacatcaggattcctaggacccctgctcgtgttacaggcggggtttttcttgttgacaagaatcctcacaataccacagagtctagactcgtggtggacttctctcaattttctagggggagcacccacgtgtcctggccaaaattcgcagtccccaacctccaatcactcaccaacctcttgtcctccaatttgtcctggttatcgctggatgtgtctgcggcgttttatcatcttcctcttcatcctgctgctatgcctcatcttcttgttggttcttctggactaccaaggtatgttgcccgtttgtcctctacttccaggaacatcaacacccagcacgggaccatgcaagacctgcacgactcctgctcaaggaacctctatgtttccctcttgttgctgtacaaaaccttcggacggaaactgcacttgtattcccatcccatcatcttgggctttcgcaagattcctatgggagtgggcctcagtccgtttctcctggctcagtttactagtgccatttgttcagtggttcgtagggctttcccccactgtttggctttcagttatatggatgatgtggtattgggggccaagtctgtacaacatcttgagtccctttttaccgctattaccaattttcttttgtctttgggtatacatttaaaccctaataaaaccaaacgttggggctactcccttaacttcatgggatatgtaattggaagttggggtactttaccacaggaacatattgtacaaaaaatcaaacaatgttttcggaaacttcctgtaaatagacctattgattggaaagtatgtcaaagaattgtgggtcttttgggctttgctgccccttttacacaatgtggctatcctgctttaatgcctttatatgcatgtatacaagctaagcaggctttcactttctcgccaacttacaaggcctttctgtgtaaacaatatctgaacctttaccccgttgctcggcaacggtcaggtctgtgccaagtgtttgctgacgcaacccccactggttggggcttggccataggccatcagcgcatgcgtggaacctttgtggctcctctgccgatccatactgcggaactcctagcagcttgttttgctcgcagccggtctggagcaaaacttatcgggactgacaactctgttgtcctctctcggaaatacacctcctttccatggctgctaggctgtgctgccaactggatcctgcgcgggacgtcctttgtctacgtcccgtcggcgctgaatcccgcggacgacccgtctcggggccgtttggggctctaccgtccccttcttcgtctgccgttccggccgaccacggggcgcacctctctttacgcggtctccccgtctgtgccttctcatctgccggaccgtgtgcacttcgcttcacctctgcacgtcgcatggagaccaccgtgaacgcccaccggaacaccctggtcaaggtcttacataagaggactcttggactctcagcaatgtcaacgaccgaccttgaggcatacttcaaagactgtgtgtttaatgactgggaggagttgggggaggagattaggttaaagcttctttgtactaggaggctgtaggcataaattggtctgttcaccagcaccatgcaactttttcacctctgcctaatcatctcatgttcatgtcctactgttcaagcctccaagctgtgccttgggtggctttggggcatggacattgacccgtataaagaatttggagcttctgtggagttactctcttttttgccttctgacttctttccttctattcgagatctcctcgacaccgcctctgctctgtatcgggaggccttagagtctccggaacattgttcacctcaccatacagcactcaggcaagctattctgtgttggggtgagttgatgaatctggccacctgggtgggaagtaatttggaagacccagcatccagggaattagtagtcagctatgtcaatgttaatatgggcctaaaaatcagacaactattgtggtttcacatttcctgtcttacttttggaagagaaactgttcttgagtatttggtgtcttttggagtgtggattcgcactcctcctgcttacagaccaccaaatgcccctatcttatcaacacttccggaaactactgttgttagacgacgaggcaggtcccctagaagaagaactccctcgcctcgcagacgaaggtctcaatcgccgcgtcgcagaagatctcaatctcgggaatctcaatgttagtatcccttggactcataaggtgggaaactttactgggctttattcttctactgtacctgtctttaatcctgagtggcaaactccctcttttcctcacattcatttgcaggaggacattattaatagatgtcaacaatatgtgggccctcttacagttaatgaaaaaaggagattaaaattaattatgcctgctaggttctatcctaaccttaccaaatatttgcccttagataaaggcattaaaccttattatcctgaacatgcagttaatcattacttccaaactaggcattatttacatactctgtggaaggcgggcattttatataagagagaaactacacgcagcgcctcattttgtgggtcaccatattcttgggaacaagagctacagcatgggaggttggtcttccaaacctcgacaaggcatggggacaaatctttctgttcccaatcctctgggattctttcccgatcaccagttggaccctgcattcggagccaactcaaacaatccagattgggacttcaaccccaacaaggatcactggccagaggcaaaccaggtaggagtgggagcattcgggccagggttcaccccaccacacggcggtcttttggggtggagccctcaggctcagggcatattgacaacagtgccagcagctcctcctcctgcctccaccaatcggcagtcaggaagacagcctactcccatctctccacctctaagagacagtcatcctcaggccatgcagtggaa

SEQ ID NO: 6 - DNA, 20 bp, synthetic construct
  source 1..20: mol_type = other DNA
caccaaactcttcaagatcc

SEQ ID NO: 7 - DNA, 20 bp, synthetic construct
  source 1..20: mol_type = other DNA
caggaacagtgagccctgct

SEQ ID NO: 8 - DNA, 20 bp, synthetic construct
  source 1..20: mol_type = other DNA
acccctgctcgtgttacagg

SEQ ID NO: 9 - DNA, 20 bp, synthetic construct
  source 1..20: mol_type = other DNA
caaaaatcctcacaatacca

SEQ ID NO: 10 - DNA, 20 bp, synthetic construct
  source 1..20: mol_type = other DNA
ccaatttgtcctggttatcg

SEQ ID NO: 11 - DNA, 20 bp, synthetic construct
  source 1..20: mol_type = other DNA
gcccgtttgtcctctaattc

SEQ ID NO: 12 - DNA, 20 bp, synthetic construct
  source 1..20: mol_type = other DNA
ggctttcgcaaaatacctat

SEQ ID NO: 13 - DNA, 20 bp, synthetic construct
  source 1..20: mol_type = other DNA
actgtctggctttcagttat

SEQ ID NO: 14 - DNA, 20 bp, synthetic construct
  source 1..20: mol_type = other DNA
ggccaagtctgtacaacatc

SEQ ID NO: 15 - DNA, 20 bp, synthetic construct
  source 1..20: mol_type = other DNA
ttaccaattttcttttgtct

SEQ ID NO: 16 - DNA, 20 bp, synthetic construct
  source 1..20: mol_type = other DNA
ccctcacaaaacaaaaagat

SEQ ID NO: 17 - DNA, 20 bp, synthetic construct
  source 1..20: mol_type = other DNA
gggatattcccttaacttca

SEQ ID NO: 18 - DNA, 20 bp, synthetic construct
  source 1..20: mol_type = other DNA
acttcatgggatatgtaatt

SEQ ID NO: 19 - DNA, 20 bp, synthetic construct
  source 1..20: mol_type = other DNA
attgattggaaagtatgtca

SEQ ID NO: 20 - DNA, 20 bp, synthetic construct
  source 1..20: mol_type = other DNA
ggaaagtatgtcaacgaatt

SEQ ID NO: 21 - DNA, 20 bp, synthetic construct
  source 1..20: mol_type = other DNA
tcaacgaattgtgggtcttt

SEQ ID NO: 22 - DNA, 20 bp, synthetic construct
  source 1..20: mol_type = other DNA
tgccgcccctttcacgcaat

SEQ ID NO: 23 - DNA, 20 bp, synthetic construct
  source 1..20: mol_type = other DNA
ctgctaggctgtgctgccaa

SEQ ID NO: 24 - DNA, 20 bp, synthetic construct
  source 1..20: mol_type = other DNA
ttgtttacgtcccgtcggcg

SEQ ID NO: 25 - DNA, 20 bp, synthetic construct
  source 1..20: mol_type = other DNA
cttcaaagactgtgtgttta

SEQ ID NO: 26 - DNA, 20 bp, synthetic construct
  source 1..20: mol_type = other DNA
ctgtgtgtttactgagtggg

SEQ ID NO: 27 - DNA, 20 bp, synthetic construct
  source 1..20: mol_type = other DNA
tcaagcctccaagctgtgcc

SEQ ID NO: 28 - DNA, 20 bp, synthetic construct
  source 1..20: mol_type = other DNA
taaagaatttggagcttctg

SEQ ID NO: 29 - DNA, 20 bp, synthetic construct
  source 1..20: mol_type = other DNA
tgctctgtatcgggaggcct

SEQ ID NO: 30 - DNA, 20 bp, synthetic construct
  source 1..20: mol_type = other DNA
tcaggcaagctattctgtgt

SEQ ID NO: 31 - DNA, 20 bp, synthetic construct
  source 1..20: mol_type = other DNA
ttctgtgttggggtgagttg

SEQ ID NO: 32 - DNA, 20 bp, synthetic construct
  source 1..20: mol_type = other DNA
gagttgatgaatctagccac

SEQ ID NO: 33 - DNA, 20 bp, synthetic construct
  source 1..20: mol_type = other DNA
tttggaagatccagcatcca

SEQ ID NO: 34 - DNA, 20 bp, synthetic construct
  source 1..20: mol_type = other DNA
ttttgggagagaaactgttc

SEQ ID NO: 35 - DNA, 20 bp, synthetic construct
  source 1..20: mol_type = other DNA
cttgaatatttggtgtcttt

SEQ ID NO: 36 - DNA, 20 bp, synthetic construct
  source 1..20: mol_type = other DNA
tatttggtgtcttttggagt

SEQ ID NO: 37 - DNA, 20 bp, synthetic construct
  source 1..20: mol_type = other DNA
aaatatttgcccttagataa

SEQ ID NO: 38 - DNA, 20 bp, synthetic construct
  source 1..20: mol_type = other DNA
atttacacactctttggaag

SEQ ID NO: 39 - DNA, 20 bp, synthetic construct
  source 1..20: mol_type = other DNA
ggcgggtatcttatataaaa

SEQ ID NO: 40 - DNA, 20 bp, synthetic construct
  source 1..20: mol_type = other DNA
cacacgtagcgcctcatttt

SEQ ID NO: 41 - DNA, 20 bp, synthetic construct
  source 1..20: mol_type = other DNA
tctttctgtccccaatcccc

SEQ ID NO: 42 - DNA, 20 bp, synthetic construct
  source 1..20: mol_type = other DNA
tggccggacgccaacaaggt

SEQ ID NO: 43 - DNA, 20 bp, synthetic construct
  source 1..20: mol_type = other DNA
gggagtgggagcattcgggc

SEQ ID NO: 44 - DNA, 20 bp, synthetic construct
  source 1..20: mol_type = other DNA
ccctccccatgggggactgt

SEQ ID NO: 45 - DNA, 20 bp, synthetic construct
  source 1..20: mol_type = other DNA
gccctgactctgggatcttg

SEQ ID NO: 46 - DNA, 20 bp, synthetic construct
  source 1..20: mol_type = other DNA
aaagtacagggccctgactc

SEQ ID NO: 47 - DNA, 20 bp, synthetic construct
  source 1..20: mol_type = other DNA
gatgttctccatgttcggta

SEQ ID NO: 48 - DNA, 20 bp, synthetic construct
  source 1..20: mol_type = other DNA
gtaacacgagcaggggtcct

SEQ ID NO: 49 - DNA, 20 bp, synthetic construct
  source 1..20: mol_type = other DNA
accccgcctgtaacacgagc

SEQ ID NO: 50 - DNA, 20 bp, synthetic construct
  source 1..20: mol_type = other DNA
tctagactctgtggtattgt

SEQ ID NO: 51 - DNA, 20 bp, synthetic construct
  source 1..20: mol_type = other DNA
aaaattgagagaagtccacc

SEQ ID NO: 52 - DNA, 20 bp, synthetic construct
  source 1..20: mol_type = other DNA
gcgaattttggccaagacac

SEQ ID NO: 53 - DNA, 20 bp, synthetic construct
  source 1..20: mol_type = other DNA
gtgactggagatttgggact

SEQ ID NO: 54 - DNA, 20 bp, synthetic construct
  source 1..20: mol_type = other DNA
aattggaggacaacaggttg

SEQ ID NO: 55 - DNA, 20 bp, synthetic construct
  source 1..20: mol_type = other DNA
aagaagatgaggcatagcag

SEQ ID NO: 56 - DNA, 20 bp, synthetic construct
  source 1..20: mol_type = other DNA
gtgctggttgttgatgatcc

SEQ ID NO: 57 - DNA, 20 bp, synthetic construct
  source 1..20: mol_type = other DNA
aacatagaggttccttgagc

SEQ ID NO: 58 - DNA, 20 bp, synthetic construct
  source 1..20: mol_type = other DNA
aaagcccaagatgatgggat

SEQ ID NO: 59 - DNA, 20 bp, synthetic construct
  source 1..20: mol_type = other DNA
tttgcgaaagcccaagatga

SEQ ID NO: 60 - DNA, 20 bp, synthetic construct
  source 1..20: mol_type = other DNA
tccccatctttttgttttgt

SEQ ID NO: 61 - DNA, 20 bp, synthetic construct
  source 1..20: mol_type = other DNA
tacatatcccatgaagttaa

SEQ ID NO: 62 - DNA, 20 bp, synthetic construct
  source 1..20: mol_type = other DNA
tgcatataaaggcattaaag

SEQ ID NO: 63 - DNA, 20 bp, synthetic construct
  source 1..20: mol_type = other DNA
accaggccgttgccgagcaa

SEQ ID NO: 64 - DNA, 20 bp, synthetic construct
  source 1..20: mol_type = other DNA
atggccaagccccaaccagt

SEQ ID NO: 65 - DNA, 20 bp, synthetic construct
  source 1..20: mol_type = other DNA
gcggctaggagttccgcagt

SEQ ID NO: 66 - DNA, 20 bp, synthetic construct
  source 1..20: mol_type = other DNA
tgcgagcaaaacaagcggct

SEQ ID NO: 67 - DNA, 20 bp, synthetic construct
  source 1..20: mol_type = other DNA
cgacagaattgtcagtcccg

SEQ ID NO: 68 - DNA, 20 bp, synthetic construct
  source 1..20: mol_type = other DNA
atacttgcgggagagcacga

SEQ ID NO: 69 - DNA, 20 bp, synthetic construct
  source 1..20: mol_type = other DNA
taaacaaaggacgtcccgcg

SEQ ID NO: 70 - DNA, 20 bp, synthetic construct
  source 1..20: mol_type = other DNA
gccccgggaggggtcgtccg

SEQ ID NO: 71 - DNA, 20 bp, synthetic construct
  source 1..20: mol_type = other DNA
gagccccaagcggccccggg

SEQ ID NO: 72 - DNA, 20 bp, synthetic construct
  source 1..20: mol_type = other DNA
cagatgagaaggcacagacg

SEQ ID NO: 73 - DNA, 20 bp, synthetic construct
  source 1..20: mol_type = other DNA
gttgacattgctgaaagtcc

SEQ ID NO: 74 - DNA, 20 bp, synthetic construct
  source 1..20: mol_type = other DNA
agaagctccaaattctttat

SEQ ID NO: 75 - DNA, 20 bp, synthetic construct
  source 1..20: mol_type = other DNA
gaggcggtgtcgaggagatc

SEQ ID NO: 76 - DNA, 20 bp, synthetic construct
  source 1..20: mol_type = other DNA
cccaacacagaatagcttgc

SEQ ID NO: 77 - DNA, 20 bp, synthetic construct
  source 1..20: mol_type = other DNA
ttcatcaactcaccccaaca

SEQ ID NO: 78 - DNA, 20 bp, synthetic construct
  source 1..20: mol_type = other DNA
gactactaattccctggatg

SEQ ID NO: 79 - DNA, 20 bp, synthetic construct
  source 1..20: mol_type = other DNA
catagctgactactaattcc

SEQ ID NO: 80 - DNA, 20 bp, synthetic construct
  source 1..20: mol_type = other DNA
ggtctatatgcaggaggagt

SEQ ID NO: 81 - DNA, 20 bp, synthetic construct
  source 1..20: mol_type = other DNA
tttggtggtctatatgcagg

SEQ ID NO: 82 - DNA, 20 bp, synthetic construct
  source 1..20: mol_type = other DNA
gaccttcgtctgcgaggcga

SEQ ID NO: 83 - DNA, 20 bp, synthetic construct
  source 1..20: mol_type = other DNA
tttcccaccttatgtgtcca

SEQ ID NO: 84 - DNA, 20 bp, synthetic construct
  source 1..20: mol_type = other DNA
attaaagcaaggtaccgtag

SEQ ID NO: 85 - DNA, 20 bp, synthetic construct
  source 1..20: mol_type = other DNA
aaaagaaggagtttgccatt

SEQ ID NO: 86 - DNA, 20 bp, synthetic construct
  source 1..20: mol_type = other DNA
aaatgaatgtcaggaaaaga

SEQ ID NO: 87 - DNA, 20 bp, synthetic construct
  source 1..20: mol_type = other DNA
tcaacaatgtcctcctgcaa

SEQ ID NO: 88 - DNA, 20 bp, synthetic construct
  source 1..20: mol_type = other DNA
gggcaaatatttagtaacat

SEQ ID NO: 89 - DNA, 20 bp, synthetic construct
  source 1..20: mol_type = other DNA
gtaatgattaactacatgct

SEQ ID NO: 90 - DNA, 20 bp, synthetic construct
  source 1..20: mol_type = other DNA
tataagatacccgccttcca

SEQ ID NO: 91 - DNA, 20 bp, synthetic construct
  source 1..20: mol_type = other DNA
atgctgtagatcttgttccc

SEQ ID NO: 92 - DNA, 20 bp, synthetic construct
  source 1..20: mol_type = other DNA
atgatcggggaagaatccca

SEQ ID NO: 93 - DNA, 20 bp, synthetic construct
  source 1..20: mol_type = other DNA
ggtccaactgatgatcgggg

SEQ ID NO: 94 - DNA, 20 bp, synthetic construct
  source 1..20: mol_type = other DNA
ttctgagttggctttgaatg

SEQ ID NO: 95 - DNA, 20 bp, synthetic construct
  source 1..20: mol_type = other DNA
ctggattttctgagttggct

SEQ ID NO: 96 - DNA, 20 bp, synthetic construct
  source 1..20: mol_type = other DNA
gaggtcccaatctggatttt

SEQ ID NO: 97 - DNA, 20 bp, synthetic construct
  source 1..20: mol_type = other DNA
gtgcgggttgaggtcccaat

SEQ ID NO: 98 - DNA, 20 bp, synthetic construct
  source 1..20: mol_type = other DNA
gtccggccagttgtccttgt

SEQ ID NO: 99 - DNA, 20 bp, synthetic construct
  source 1..20: mol_type = other DNA
ggggaggggtgaaccctggc

SEQ ID NO: 100 - DNA, 20 bp, synthetic construct
  source 1..20: mol_type = other DNA
cccaacagtcccccatgggg

SEQ ID NO: 101 - DNA, 20 bp, synthetic construct
  source 1..20: mol_type = other DNA
gaggagctgctggcacagtt

SEQ ID NO: 102 - DNA, 20 bp, synthetic construct
  source 1..20: mol_type = other DNA
tcccttagaggtggagataa

SEQ ID NO: 103 - DNA, 20 bp, synthetic construct
  source 1..20: mol_type = other DNA
caccaagctctgctagatcc

SEQ ID NO: 104 - DNA, 20 bp, synthetic construct
  source 1..20: mol_type = other DNA
gaacatggagaacacaacat

SEQ ID NO: 105 - DNA, 20 bp, synthetic construct
  source 1..20: mol_type = other DNA
gcggggtttttcttgttgac

SEQ ID NO: 106 - DNA, 20 bp, synthetic construct
  source 1..20: mol_type = other DNA
caagaatcctcacaatacca

SEQ ID NO: 107 - DNA, 20 bp, synthetic construct
  source 1..20: mol_type = other DNA
ccaatttgtcctggctatcg

SEQ ID NO: 108 - DNA, 20 bp, synthetic construct
  source 1..20: mol_type = other DNA
aacctcgacaaggcatgggg

SEQ ID NO: 109 - DNA, 20 bp, synthetic construct
  source 1..20: mol_type = other DNA
ggctttcgcaagattcctat

SEQ ID NO: 110 - DNA, 20 bp, synthetic construct
  source 1..20: mol_type = other DNA
actgtttggctttcagttat

SEQ ID NO: 111 - DNA, 20 bp, synthetic construct
  source 1..20: mol_type = other DNA
gggctactcccttaacttca

SEQ ID NO: 112 - DNA, 20 bp, synthetic construct
  source 1..20: mol_type = other DNA
tgggatatgtaattggaagt

SEQ ID NO: 113 - DNA, 20 bp, synthetic construct
  source 1..20: mol_type = other DNA
ggaaagtatgtcaaagaatt

SEQ ID NO: 114 - DNA, 20 bp, synthetic construct
  source 1..20: mol_type = other DNA
gtttgctgacgcaaccccca

SEQ ID NO: 115 - DNA, 20 bp, synthetic construct
  source 1..20: mol_type = other DNA
cacctcctttccatggctgc

SEQ ID NO: 116 - DNA, 20 bp, synthetic construct
  source 1..20: mol_type = other DNA
ctgctagggtgtgctgccaa

SEQ ID NO: 117 - DNA, 20 bp, synthetic construct
  source 1..20: mol_type = other DNA
ttgtctacgtcccgtcggcg

SEQ ID NO: 118 - DNA, 20 bp, synthetic construct
  source 1..20: mol_type = other DNA
ctgtttgtttaaagactggg

SEQ ID NO: 119 - DNA, 20 bp, synthetic construct
  source 1..20: mol_type = other DNA
catggacattgacccgtata

SEQ ID NO: 120 - DNA, 20 bp, synthetic construct
  source 1..20: mol_type = other DNA
gagttgatgaatctggccac

SEQ ID NO: 121 - DNA, 20 bp, synthetic construct
  source 1..20: mol_type = other DNA
tttggaagacccagcatcca

SEQ ID NO: 122 - DNA, 20 bp, synthetic construct
  source 1..20: mol_type = other DNA
ttttggaagagaaactgttc

SEQ ID NO: 123 - DNA, 20 bp, synthetic construct
  source 1..20: mol_type = other DNA
cttgagtatttggtgtcttt

SEQ ID NO: 124 - DNA, 20 bp, synthetic construct
  source 1..20: mol_type = other DNA
tcgcagaagatctcaatctc

SEQ ID NO: 125 - DNA, 20 bp, synthetic construct
  source 1..20: mol_type = other DNA
tactgtacctgtctttaatc

SEQ ID NO: 126 - DNA, 20 bp, synthetic construct
  source 1..20: mol_type = other DNA
tacacgcagcgcctcatttt

SEQ ID NO: 127 - DNA, 20 bp, synthetic construct
  source 1..20: mol_type = other DNA
tctttctgttcccaatcctc

SEQ ID NO: 128 - DNA, 20 bp, synthetic construct
  source 1..20: mol_type = other DNA
attgggacttcaaccccaac

SEQ ID NO: 129 - DNA, 20 bp, synthetic construct
  source 1..20: mol_type = other DNA
aggagcgggagcattcgggc

SEQ ID NO: 130 - DNA, 20 bp, synthetic construct
  source 1..20: mol_type = other DNA
tgggatctagcagagcttgg

SEQ ID NO: 131 - DNA, 20 bp, synthetic construct
  source 1..20: mol_type = other DNA
aaagtataggcccctcactc

SEQ ID NO: 132 - DNA, 20 bp, synthetic construct
  source 1..20: mol_type = other DNA
gggtgaggcagtagtcggaa

SEQ ID NO: 133 - DNA, 20 bp, synthetic construct
  source 1..20: mol_type = other DNA
tcctcgagaagattgacgat

SEQ ID NO: 134 - DNA, 20 bp, synthetic construct
  source 1..20: mol_type = other DNA
tgtgttctccatgttcggtg

SEQ ID NO: 135 - DNA, 20 bp, synthetic construct
  source 1..20: mol_type = other DNA
gcgaattttggccaggacac

SEQ ID NO: 136 - DNA, 20 bp, synthetic construct
  source 1..20: mol_type = other DNA
gtgattggaggttggggact

SEQ ID NO: 137 - DNA, 20 bp, synthetic construct
  source 1..20: mol_type = other DNA
aattggaggacaagaggttg

SEQ ID NO: 138 - DNA, 20 bp, synthetic construct
  source 1..20: mol_type = other DNA
ggcatagcagcaggatgaag

SEQ ID NO: 139 - DNA, 20 bp, synthetic construct
  source 1..20: mol_type = other DNA
aaagcccaggatgatgggat

SEQ ID NO: 140 - DNA, 20 bp, synthetic construct
  source 1..20: mol_type = other DNA
cttgcgaaagcccaggatga

SEQ ID NO: 141 - DNA, 20 bp, synthetic construct
  source 1..20: mol_type = other DNA
ataggaatcttgcgaaagcc

SEQ ID NO: 142 - DNA, 20 bp, synthetic construct
  source 1..20: mol_type = other DNA
ggactgaggcccactcccat

SEQ ID NO: 143 - DNA, 20 bp, synthetic construct
  source 1..20: mol_type = other DNA
gccccaacgtttggttttat

SEQ ID NO: 144 - DNA, 20 bp, synthetic construct
  source 1..20: mol_type = other DNA
tgcatataaaggcattaagg

SEQ ID NO: 145 - DNA, 20 bp, synthetic construct
  source 1..20: mol_type = other DNA
acctgaccgttgccgggcaa

SEQ ID NO: 146 - DNA, 20 bp, synthetic construct
  source 1..20: mol_type = other DNA
atggccaagccccatccagt

SEQ ID NO: 147 - DNA, 20 bp, synthetic construct
  source 1..20: mol_type = other DNA
gctgctaggagttccgcagt

SEQ ID NO: 148 - DNA, 20 bp, synthetic construct
  source 1..20: mol_type = other DNA
tgcgagcaaaacaagctgct

SEQ ID NO: 149 - DNA, 20 bp, synthetic construct
  source 1..20: mol_type = other DNA
gtatttccgagagaggacaa

SEQ ID NO: 150 - DNA, 20 bp, synthetic construct
  source 1..20: mol_type = other DNA
tagacaaaggacgtcccgcg

SEQ ID NO: 151 - DNA, 20 bp, synthetic construct
  source 1..20: mol_type = other DNA
gccccgagacgggtcgtccg

SEQ ID NO: 152 - DNA, 20 bp, synthetic construct
  source 1..20: mol_type = other DNA
gagtcccaaacggccccgag

SEQ ID NO: 153 - DNA, 20 bp, synthetic construct
  source 1..20: mol_type = other DNA
gcagatgaagaaggggacgg

SEQ ID NO: 154 - DNA, 20 bp, synthetic construct
  source 1..20: mol_type = other DNA
gttgacattgctgagagtcc

SEQ ID NO: 155 - DNA, 20 bp, synthetic construct
  source 1..20: mol_type = other DNA
ggtcggtcgttgacattgct

SEQ ID NO: 156 - DNA, 20 bp, synthetic construct
  source 1..20: mol_type = other DNA
ggtctgtaagcgggaggagt

SEQ ID NO: 157 - DNA, 20 bp, synthetic construct
  source 1..20: mol_type = other DNA
tttggtggtctgtaagcggg

SEQ ID NO: 158 - DNA, 20 bp, synthetic construct
  source 1..20: mol_type = other DNA
gtttgagttggctccgaacg

SEQ ID NO: 159 - DNA, 20 bp, synthetic construct
  source 1..20: mol_type = other DNA
tttcccaccttatgagtcca

SEQ ID NO: 160 - DNA, 20 bp, synthetic construct
  source 1..20: mol_type = other DNA
ccagtaaagtttcccacctt

SEQ ID NO: 161 - DNA, 20 bp, synthetic construct
  source 1..20: mol_type = other DNA
attaaagacaggtacagtag

SEQ ID NO: 162 - DNA, 20 bp, synthetic construct
  source 1..20: mol_type = other DNA
aaaggagggagtttgccact

SEQ ID NO: 163 - DNA, 20 bp, synthetic construct
  source 1..20: mol_type = other DNA
aaatgaatgtgaggaaagga

SEQ ID NO: 164 - DNA, 20 bp, synthetic construct
  source 1..20: mol_type = other DNA
ttaataatgtcctcctgtaa

SEQ ID NO: 165 - DNA, 20 bp, synthetic construct
  source 1..20: mol_type = other DNA
gggcaaatatttggtaaggt

SEQ ID NO: 166 - DNA, 20 bp, synthetic construct
  source 1..20: mol_type = other DNA
gtaatgattaactgcatgtt

SEQ ID NO: 167 - DNA, 20 bp, synthetic construct
  source 1..20: mol_type = other DNA
tatagaatgccagccttcca

SEQ ID NO: 168 - DNA, 20 bp, synthetic construct
  source 1..20: mol_type = other DNA
cgtgtagtttctctcttata

SEQ ID NO: 169 - DNA, 20 bp, synthetic construct
  source 1..20: mol_type = other DNA
atgctgtagctcttgttccc

SEQ ID NO: 170 - DNA, 20 bp, synthetic construct
  source 1..20: mol_type = other DNA
gtgatcgggaaagaatccca

SEQ ID NO: 171 - DNA, 20 bp, synthetic construct
  source 1..20: mol_type = other DNA
ggtccaactggtgatcggga

SEQ ID NO: 172 - DNA, 20 bp, synthetic construct
  source 1..20: mol_type = other DNA
gaagtcccaatctggattgt

SEQ ID NO: 173 - DNA, 20 bp, synthetic construct
  source 1..20: mol_type = other DNA
gttggggttgaagtcccaat

SEQ ID NO: 174 - DNA, 20 bp, synthetic construct
  source 1..20: mol_type = other DNA
ctctggccagtgatccttgt

SEQ ID NO: 175 - DNA, 20 bp, synthetic construct
  source 1..20: mol_type = other DNA
gtggtggggtgaaccctggc

SEQ ID NO: 176 - DNA, 20 bp, synthetic construct
  source 1..20: mol_type = other DNA
tctcttagaggtggagagat

SEQ ID NO: 177 - DNA, 20 bp, synthetic construct
  source 1..20: mol_type = other DNA
ctgccttccaccaagctctg

SEQ ID NO: 178 - DNA, 20 bp, synthetic construct
  source 1..20: mol_type = other DNA
caccaagctctgcaggatcc

SEQ ID NO: 179 - DNA, 20 bp, synthetic construct
  source 1..20: mol_type = other DNA
ctctgcaggatcccagagtc

SEQ ID NO: 180 - DNA, 20 bp, synthetic construct
  source 1..20: mol_type = other DNA
caggaacagtaaaccctgct

SEQ ID NO: 181 - DNA, 20 bp, synthetic construct
  source 1..20: mol_type = other DNA
gaacatggagaacatcacat

SEQ ID NO: 182 - DNA, 20 bp, synthetic construct
  source 1..20: mol_type = other DNA
gaagttggggaacattgcca

SEQ ID NO: 183 - DNA, 20 bp, synthetic construct
  source 1..20: mol_type = other DNA
caagaatcctcacaataccg

SEQ ID NO: 184 - DNA, 20 bp, synthetic construct
  source 1..20: mol_type = other DNA
gacttctctcaattttctag

SEQ ID NO: 185 - DNA, 20 bp, synthetic construct
  source 1..20: mol_type = other DNA
tcatcttcttattggttctt

SEQ ID NO: 186 - DNA, 20 bp, synthetic construct
  source 1..20: mol_type = other DNA
aacctcgcaaaggcatgggg

SEQ ID NO: 187 - DNA, 20 bp, synthetic construct
  source 1..20: mol_type = other DNA
catgttgctgtacaaaacct

SEQ ID NO: 188 - DNA, 20 bp, synthetic construct
  source 1..20: mol_type = other DNA
actgtttggctttcagctat

SEQ ID NO: 189 - DNA, 20 bp, synthetic construct
  source 1..20: mol_type = other DNA
ggccaagtctgtacagcatc

SEQ ID NO: 190 - DNA, 20 bp, synthetic construct
  source 1..20: mol_type = other DNA
accctaacaaaacaaaaaga

SEQ ID NO: 191 - DNA, 20 bp, synthetic construct
  source 1..20: mol_type = other DNA
ggggttattccctaaacttc

SEQ ID NO: 192 - DNA, 20 bp, synthetic construct
  source 1..20: mol_type = other DNA
tgctgctccatttacacaat

SEQ ID NO: 193 - DNA, 20 bp, synthetic construct
  source 1..20: mol_type = other DNA
ctgctaggctgtactgccaa

SEQ ID NO: 194 - DNA, 20 bp, synthetic construct
  source 1..20: mol_type = other DNA
catggacattgacccttata

SEQ ID NO: 195 - DNA, 20 bp, synthetic construct
  source 1..20: mol_type = other DNA
taaagaatttggagctactg

SEQ ID NO: 196 - DNA, 20 bp, synthetic construct
  source 1..20: mol_type = other DNA
agctctgtatcgggaagcct

SEQ ID NO: 197 - DNA, 20 bp, synthetic construct
  source 1..20: mol_type = other DNA
gcaagccattctctgctggg

SEQ ID NO: 198 - DNA, 20 bp, synthetic construct
  source 1..20: mol_type = other DNA
gaattgatgactctagctac

SEQ ID NO: 199 - DNA, 20 bp, synthetic construct
  source 1..20: mol_type = other DNA
atttggaagatccagcatcc

SEQ ID NO: 200 - DNA, 20 bp, synthetic construct
  source 1..20: mol_type = other DNA
tcaattatgttaatactaac

SEQ ID NO: 201 - DNA, 20 bp, synthetic construct
  source 1..20: mol_type = other DNA
ttttggaagagagactgtac

SEQ ID NO: 202 - DNA, 20 bp, synthetic construct
  source 1..20: mol_type = other DNA
cttgaatatttggtctcttt

SEQ ID NO: 203 - DNA, 20 bp, synthetic construct
  source 1..20: mol_type = other DNA
tatttggtctctttcggagt

SEQ ID NO: 204 - DNA, 20 bp, synthetic construct
  source 1..20: mol_type = other DNA
tacagtacctatctttaatc

SEQ ID NO: 205 - DNA, 20 bp, synthetic construct
  source 1..20: mol_type = other DNA
aaatatttgcccttagacaa

SEQ ID NO: 206 - DNA, 20 bp, synthetic construct
  source 1..20: mol_type = other DNA
cacacgtagcgcatcatttt

SEQ ID NO: 207 - DNA, 20 bp, synthetic construct
  source 1..20: mol_type = other DNA
tctttctgttcccaaccctc

SEQ ID NO: 208 - DNA, 20 bp, synthetic construct
  source 1..20: mol_type = other DNA
tggccagcagccaaccaggt

SEQ ID NO: 209 - DNA, 20 bp, synthetic construct
  source 1..20: mol_type = other DNA
aggagtgggagcattcgggc

SEQ ID NO: 210 - DNA, 20 bp, synthetic construct
  source 1..20: mol_type = other DNA
ccctccacacggcggtgttt

SEQ ID NO: 211 - DNA, 20 bp, synthetic construct
  source 1..20: mol_type = other DNA
aaaatacagacccctgactc

SEQ ID NO: 212 - DNA, 20 bp, synthetic construct
  source 1..20: mol_type = other DNA
gtgagaggcaatattcggag

SEQ ID NO: 213 - DNA, 20 bp, synthetic construct
  source 1..20: mol_type = other DNA
gatgttctccatgttcgtca

SEQ ID NO: 214 - DNA, 20 bp, synthetic construct
  source 1..20: mol_type = other DNA
tctagactctgcggtattgt

SEQ ID NO: 215 - DNA, 20 bp, synthetic construct
  source 1..20: mol_type = other DNA
aattggaggacaggaggttg

SEQ ID NO: 216 - DNA, 20 bp, synthetic construct
  source 1..20: mol_type = other DNA
gtactggttgttgttgatcc

SEQ ID NO: 217 - DNA, 20 bp, synthetic construct
  source 1..20: mol_type = other DNA
aacatagagttgccttgagc

SEQ ID NO: 218 - DNA, 20 bp, synthetic construct
  source 1..20: mol_type = other DNA
acagcaacatgagggaaaca

SEQ ID NO: 219 - DNA, 20 bp, synthetic construct
  source 1..20: mol_type = other DNA
gtttgagttggctccgaatg

SEQ ID NO: 220 - DNA, 20 bp, synthetic construct
  source 1..20: mol_type = other DNA
aaagcccaggacgatgggat

SEQ ID NO: 221 - DNA, 20 bp, synthetic construct
  source 1..20: mol_type = other DNA
tttgcgaaagcccaggacga

SEQ ID NO: 222 - DNA, 20 bp, synthetic construct
  source 1..20: mol_type = other DNA
accccatctttttgttttgt

SEQ ID NO: 223 - DNA, 20 bp, synthetic construct
  source 1..20: mol_type = other DNA
tatgtaacccatgaagttta

SEQ ID NO: 224 - DNA, 20 bp, synthetic construct
  source 1..20: mol_type = other DNA
tgcatacaaaggcattaagg

SEQ ID NO: 225 - DNA, 20 bp, synthetic construct
  source 1..20: mol_type = other DNA
atggccaagccccagccagt

SEQ ID NO: 226 - DNA, 20 bp, synthetic construct
  source 1..20: mol_type = other DNA
atatttccgcgagaggacga

SEQ ID NO: 227 - DNA, 20 bp, synthetic construct
  source 1..20: mol_type = other DNA
gccccgagaggggtcgtccg

SEQ ID NO: 228 - DNA, 20 bp, synthetic construct
  source 1..20: mol_type = other DNA
gagtcccaagcggccccgag

SEQ ID NO: 229 - DNA, 20 bp, synthetic construct
  source 1..20: mol_type = other DNA
gcagacggagaaggggacga

SEQ ID NO: 230 - DNA, 20 bp, synthetic construct
  source 1..20: mol_type = other DNA
tctaagggcaaatatttagt

SEQ ID NO: 231 - DNA, 20 bp, synthetic construct
  source 1..20: mol_type = other DNA
ctcttatgtaagaccttggg

SEQ ID NO: 232 - DNA, 20 bp, synthetic construct
  source 1..20: mol_type = other DNA
gttgacattgctgggagtcc

SEQ ID NO: 233 - DNA, 20 bp, synthetic construct
  source 1..20: mol_type = other DNA
agtagctccaaattctttat

SEQ ID NO: 234 - DNA, 20 bp, synthetic construct
  source 1..20: mol_type = other DNA
agaagtcagaaggcaaaaac

SEQ ID NO: 235 - DNA, 20 bp, synthetic construct
  source 1..20: mol_type = other DNA
cccagcagagaatggcttgc

SEQ ID NO: 236 - DNA, 20 bp, synthetic construct
  source 1..20: mol_type = other DNA
gtcatcaattccccccagca

SEQ ID NO: 237 - DNA, 20 bp, synthetic construct
  source 1..20: mol_type = other DNA
ttattacccacccaggtagc

SEQ ID NO: 238 - DNA, 20 bp, synthetic construct
  source 1..20: mol_type = other DNA
gactactagatccctggatg

SEQ ID NO: 239 - DNA, 20 bp, synthetic construct
  source 1..20: mol_type = other DNA
cataattgactactagatcc

SEQ ID NO: 240 - DNA, 20 bp, synthetic construct
  source 1..20: mol_type = other DNA
ggtctataggctggaggagt

SEQ ID NO: 241 - DNA, 20 bp, synthetic construct
  source 1..20: mol_type = other DNA
tttggtggtctataggctgg

SEQ ID NO: 242 - DNA, 20 bp, synthetic construct
  source 1..20: mol_type = other DNA
gatctgcgtctgcgaggcga

SEQ ID NO: 243 - DNA, 20 bp, synthetic construct
  source 1..20: mol_type = other DNA
attaaagataggtactgtag

SEQ ID NO: 244 - DNA, 20 bp, synthetic construct
  source 1..20: mol_type = other DNA
aaaggaaggagtttgccatt

SEQ ID NO: 245 - DNA, 20 bp, synthetic construct
  source 1..20: mol_type = other DNA
aaatgaatcttaggaaagga

SEQ ID NO: 246 - DNA, 20 bp, synthetic construct
  source 1..20: mol_type = other DNA
ttaataatgtcctcttgtaa

SEQ ID NO: 247 - DNA, 20 bp, synthetic construct
  source 1..20: mol_type = other DNA
aatatttagtgtgggtagga

SEQ ID NO: 248 - DNA, 20 bp, synthetic construct
  source 1..20: mol_type = other DNA
gtaatgattaactacctgat

SEQ ID NO: 249 - DNA, 20 bp, synthetic construct
  source 1..20: mol_type = other DNA
tatagaataccagccttcca

SEQ ID NO: 250 - DNA, 20 bp, synthetic construct
  source 1..20: mol_type = other DNA
cgtgtggtttccctcttata

SEQ ID NO: 251 - DNA, 20 bp, synthetic construct
  source 1..20: mol_type = other DNA
atgatcgggaaagaatccca

SEQ ID NO: 252 - DNA, 20 bp, synthetic construct
  source 1..20: mol_type = other DNA
ggtccaactgatgatcggga

SEQ ID NO: 253 - DNA, 20 bp, synthetic construct
  source 1..20: mol_type = other DNA
ctggattgtttgagttggct

SEQ ID NO: 254 - DNA, 20 bp, synthetic construct
  source 1..20: mol_type = other DNA
gatggggttgaagtcccaat

SEQ ID NO: 255 - DNA, 20 bp, synthetic construct
  source 1..20: mol_type = other DNA
tgctggccagtggtccttga

SEQ ID NO: 256 - DNA, 20 bp, synthetic construct
  source 1..20: mol_type = other DNA
gtggaggggtgaaccctggc

SEQ ID NO: 257 - DNA, 20 bp, synthetic construct
  source 1..20: mol_type = other DNA
cccaaaacaccgccgtgtgg

SEQ ID NO: 258 - DNA, 20 bp, synthetic construct
  source 1..20: mol_type = other DNA
cgattggtggaggcaggagg

SEQ ID NO: 259 - DNA, 20 bp, synthetic construct
  source 1..20: mol_type = other DNA
gaacatggagaacaccacat

SEQ ID NO: 260 - DNA, 20 bp, synthetic construct
  source 1..20: mol_type = other DNA
ctgtgtgtttaatgactggg

SEQ ID NO: 261 - DNA, 20 bp, synthetic construct
  source 1..20: mol_type = other DNA
tggccagaggcaaaccaggt

SEQ ID NO: 262 - DNA, 20 bp, synthetic construct
  source 1..20: mol_type = other DNA
gggagaggcagtagtcggaa

SEQ ID NO: 263 - DNA, 20 bp, synthetic construct
  source 1..20: mol_type = other DNA
ggtgttctccatgttcggtg

SEQ ID NO: 264 - DNA, 20 bp, synthetic construct
  source 1..20: mol_type = other DNA
cttgcgaaagcccaagatga

SEQ ID NO: 265 - DNA, 20 bp, synthetic construct
  source 1..20: mol_type = other DNA
acctgaccgttgccgagcaa

SEQ ID NO: 266 - DNA, 20 bp, synthetic construct
  source 1..20: mol_type = other DNA
gagccccaaacggccccgag

SEQ ID NO: 267 - DNA, 20 bp, synthetic construct
  source 1..20: mol_type = other DNA
ggtctgtaagcaggaggagt

SEQ ID NO: 268 - DNA, 20 bp, synthetic construct
  source 1..20: mol_type = other DNA
tttggtggtctgtaagcagg

SEQ ID NO: 269 - DNA, 20 bp, synthetic construct
  source 1..20: mol_type = other DNA
aaaagagggagtttgccact

SEQ ID NO: 270 - DNA, 20 bp, synthetic construct
  source 1..20: mol_type = other DNA
aaatgaatgtgaggaaaaga

SEQ ID NO: 271 - DNA, 20 bp, synthetic construct
  source 1..20: mol_type = other DNA
ttaataatgtcctcctgcaa

SEQ ID NO: 272 - DNA, 20 bp, synthetic construct
  source 1..20: mol_type = other DNA
tataaaatgcccgccttcca

SEQ ID NO: 273 - DNA, 22 bp, synthetic construct
  source 1..22: mol_type = other DNA
cctctgcacgtcgcatggagac

SEQ ID NO: 274 - DNA, 22 bp, synthetic construct
  source 1..22: mol_type = other DNA
cccactgtctggctttcagtta

SEQ ID NO: 275 - DNA, 22 bp, synthetic construct
  source 1..22: mol_type = other DNA
gagttggctttgaatgcagggt

SEQ ID NO: 276 - DNA, 22 bp, synthetic construct
  source 1..22: mol_type = other DNA
ccatgttcggtacagggtcccc

SEQ ID NO: 277 - DNA, 22 bp, synthetic construct
  source 1..22: mol_type = other DNA
gatactgtttacttagaaaggc

SEQ ID NO: 278 - DNA, 22 bp, synthetic construct
  source 1..22: mol_type = other DNA
catttcctgtcttacttttggg

SEQ ID NO: 279 - DNA, 22 bp, synthetic construct
  source 1..22: mol_type = other DNA
tgttgacaaaaatcctcacaat

SEQ ID NO: 280 - DNA, 22 bp, synthetic construct
  source 1..22: mol_type = other DNA
catctgccggaccgtgtgcact

SEQ ID NO: 281 - DNA, 22 bp, synthetic construct
  source 1..22: mol_type = other DNA
tctggactatcaaggtatgttg

SEQ ID NO: 282 - DNA, 22 bp, synthetic construct
  source 1..22: mol_type = other DNA
catcccatcatcttgggctttc

SEQ ID NO: 283 - DNA, 22 bp, synthetic construct
  source 1..22: mol_type = other DNA
gcaatgtcaacgaccgaccttg

SEQ ID NO: 284 - DNA, 22 bp, synthetic construct
  source 1..22: mol_type = other DNA
gcagtatggatcggcagaggag

SEQ ID NO: 285 - DNA, 22 bp, synthetic construct
  source 1..22: mol_type = other DNA
tgtggcaatgtgccccaactcc

SEQ ID NO: 286 - DNA, 22 bp, synthetic construct
  source 1..22: mol_type = other DNA
cagtcccaaatctccagtcact

SEQ ID NO: 287 - DNA, 22 bp, synthetic construct
  source 1..22: mol_type = other DNA
cactcctcctgcatatagacca

SEQ ID NO: 288 - DNA, 22 bp, synthetic construct
  source 1..22: mol_type = other DNA
tgttggttcttctggactatca

SEQ ID NO: 289 - DNA, 22 bp, synthetic construct
  source 1..22: mol_type = other DNA
caccttatgtgtccaaggaata

SEQ ID NO: 290 - DNA, 22 bp, synthetic construct
  source 1..22: mol_type = other DNA
tctgcgaggcgagggagttctt

SEQ ID NO: 291 - DNA, 22 bp, synthetic construct
  source 1..22: mol_type = other DNA
gcgccgacgggacgtaaacaaa

SEQ ID NO: 292 - DNA, 22 bp, synthetic construct
  source 1..22: mol_type = other DNA
cacccaggtggctagattcatc

SEQ ID NO: 293 - DNA, 22 bp, synthetic construct
  source 1..22: mol_type = other DNA
tctgcatcctgctgctatgcct

SEQ ID NO: 294 - DNA, 22 bp, synthetic construct
  source 1..22: mol_type = other DNA
tctacggtaccttgctttaatc

SEQ ID NO: 295 - DNA, 22 bp, synthetic construct
  source 1..22: mol_type = other DNA
caaaatacctatgggagtgggc

SEQ ID NO: 296 - DNA, 22 bp, synthetic construct
  source 1..22: mol_type = other DNA
attcgagatctcctcgacaccg

SEQ ID NO: 297 - DNA, 22 bp, synthetic construct
  source 1..22: mol_type = other DNA
ggaacagtgagccctgctcaga

SEQ ID NO: 298 - DNA, 22 bp, synthetic construct
  source 1..22: mol_type = other DNA
gcgacgcggcgattgagacctt

SEQ ID NO: 299 - DNA, 22 bp, synthetic construct
  source 1..22: mol_type = other DNA
tgtcttacttttgggagagaaa

SEQ ID NO: 300 - DNA, 22 bp, synthetic construct
  source 1..22: mol_type = other DNA
cccctccccatgggggactgtt

SEQ ID NO: 301 - DNA, 22 bp, synthetic construct
  source 1..22: mol_type = other DNA
ccctagaaaattgagagaagtc

SEQ ID NO: 302 - DNA, 22 bp, synthetic construct
  source 1..22: mol_type = other DNA
cttaacttcatgggatatgtaa

SEQ ID NO: 303 - DNA, 22 bp, synthetic construct
  source 1..22: mol_type = other DNA
cttcacctctgcacgtcgcatg

SEQ ID NO: 304 - DNA, 22 bp, synthetic construct
  source 1..22: mol_type = other DNA
tgctggtggctccagttcagga

SEQ ID NO: 305 - DNA, 22 bp, synthetic construct
  source 1..22: mol_type = other DNA
ctcccaaaagtaagacaggaaa

SEQ ID NO: 306 - DNA, 22 bp, synthetic construct
  source 1..22: mol_type = other DNA
ctcatgttgctgtacaaaacct

SEQ ID NO: 307 - DNA, 22 bp, synthetic construct
  source 1..22: mol_type = other DNA
cttggctcagtttactagtgcc

SEQ ID NO: 308 - DNA, 22 bp, synthetic construct
  source 1..22: mol_type = other DNA
ctcaattttctagggggaacac

SEQ ID NO: 309 - DNA, 22 bp, synthetic construct
  source 1..22: mol_type = other DNA
tgactgccgattggtggaggca

SEQ ID NO: 310 - DNA, 22 bp, synthetic construct
  source 1..22: mol_type = other DNA
cggtggtctccatgcgacgtgc

SEQ ID NO: 311 - DNA, 22 bp, synthetic construct
  source 1..22: mol_type = other DNA
tgggaacaagatctacagcatg

SEQ ID NO: 312 - DNA, 22 bp, synthetic construct
  source 1..22: mol_type = other DNA
tttgtctttgggtatacattta

SEQ ID NO: 313 - DNA, 22 bp, synthetic construct
  source 1..22: mol_type = other DNA
cgccaacttacaaggcctttct

SEQ ID NO: 314 - DNA, 22 bp, synthetic construct
  source 1..22: mol_type = other DNA
cgcaatgtggatatcctgcttt

SEQ ID NO: 315 - DNA, 22 bp, synthetic construct
  source 1..22: mol_type = other DNA
tgggatatgtaattgggagttg

SEQ ID NO: 316 - DNA, 22 bp, synthetic construct
  source 1..22: mol_type = other DNA
tgacattcatttgcaggaggac

SEQ ID NO: 317 - DNA, 22 bp, synthetic construct
  source 1..22: mol_type = other DNA
tgtaaacaggcctattgattgg

SEQ ID NO: 318 - DNA, 22 bp, synthetic construct
  source 1..22: mol_type = other DNA
cgagattgagatcttctgcgac

SEQ ID NO: 319 - DNA, 22 bp, synthetic construct
  source 1..22: mol_type = other DNA
tgaatatttggtgtcttttgga

SEQ ID NO: 320 - DNA, 22 bp, synthetic construct
  source 1..22: mol_type = other DNA
tgaactggagccaccagcagga

SEQ ID NO: 321 - DNA, 22 bp, synthetic construct
  source 1..22: mol_type = other DNA
gacttctttccttctattcgag

SEQ ID NO: 322 - DNA, 22 bp, synthetic construct
  source 1..22: mol_type = other DNA
tcaactcaccccaacacagaat

SEQ ID NO: 323 - DNA, 22 bp, synthetic construct
  source 1..22: mol_type = other DNA
cctcaccatacggcactcaggc

SEQ ID NO: 324 - DNA, 22 bp, synthetic construct
  source 1..22: mol_type = other DNA
gagcagggctcactgttcctga

SEQ ID NO: 325 - DNA, 22 bp, synthetic construct
  source 1..22: mol_type = other DNA
tgtcctactgttcaagcctcca

SEQ ID NO: 326 - DNA, 22 bp, synthetic construct
  source 1..22: mol_type = other DNA
ccgcctgttgtaccgaccgacc

SEQ ID NO: 327 - DNA, 22 bp, synthetic construct
  source 1..22: mol_type = other DNA
atggctgctaggctgtgctgcc

SEQ ID NO: 328 - DNA, 22 bp, synthetic construct
  source 1..22: mol_type = other DNA
ggaagtgttgataagatagggg

SEQ ID NO: 329 - DNA, 22 bp, synthetic construct
  source 1..22: mol_type = other DNA
caagaatatggtgacccgcaaa

SEQ ID NO: 330 - DNA, 22 bp, synthetic construct
  source 1..22: mol_type = other DNA
gtccgtaggttttgtacagcaa

SEQ ID NO: 331 - DNA, 22 bp, synthetic construct
  source 1..22: mol_type = other DNA
acgcatgcgctgatggcctatg

SEQ ID NO: 332 - DNA, 22 bp, synthetic construct
  source 1..22: mol_type = other DNA
ttggacacataaggtgggaaac

SEQ ID NO: 333 - DNA, 22 bp, synthetic construct
  source 1..22: mol_type = other DNA
gtccccaatcccctgggattct

SEQ ID NO: 334 - DNA, 22 bp, synthetic construct
  source 1..22: mol_type = other DNA
ggagactctaaggcctcccgat

SEQ ID NO: 335 - DNA, 22 bp, synthetic construct
  source 1..22: mol_type = other DNA
accaaactcttcaagatcccag

SEQ ID NO: 336 - DNA, 22 bp, synthetic construct
  source 1..22: mol_type = other DNA
gtcgtgctctcccgcaagtata

SEQ ID NO: 337 - DNA, 22 bp, synthetic construct
  source 1..22: mol_type = other DNA
gtggttcgtagggctttccccc

SEQ ID NO: 338 - DNA, 22 bp, synthetic construct
  source 1..22: mol_type = other DNA
gtgttggggtgagttgatgaat

SEQ ID NO: 339 - DNA, 22 bp, synthetic construct
  source 1..22: mol_type = other DNA
aatcaataggcctgtttacagg

SEQ ID NO: 340 - DNA, 22 bp, synthetic construct
  source 1..22: mol_type = other DNA
aagtaaacagtatctgaacctt

SEQ ID NO: 341 - DNA, 22 bp, synthetic construct
  source 1..22: mol_type = other DNA
gtacagggtccccagtcttcga

SEQ ID NO: 342 - DNA, 22 bp, synthetic construct
  source 1..22: mol_type = other DNA
gttatatggatgatgtggtttt

SEQ ID NO: 343 - DNA, 22 bp, synthetic construct
  source 1..22: mol_type = other DNA
tccccgatcatcagttggaccc

SEQ ID NO: 344 - DNA, 22 bp, synthetic construct
  source 1..22: mol_type = other DNA
aagactgtgtgtttactgagtg

SEQ ID NO: 345 - DNA, 22 bp, synthetic construct
  source 1..22: mol_type = other DNA
tttactgtaaggggccccacaa

SEQ ID NO: 346 - DNA, 22 bp, synthetic construct
  source 1..22: mol_type = other DNA
tttcctgacattcatttgcagg

SEQ ID NO: 347 - DNA, 22 bp, synthetic construct
  source 1..22: mol_type = other DNA
aaattacttcccacccaggtgg

SEQ ID NO: 348 - DNA, 22 bp, synthetic construct
  source 1..22: mol_type = other DNA
aaagagtgtgtaaataatgtct

SEQ ID NO: 349 - DNA, 22 bp, synthetic construct
  source 1..22: mol_type = other DNA
tttgcaggaggacattgttgat

SEQ ID NO: 350 - DNA, 22 bp, synthetic construct
  source 1..22: mol_type = other DNA
taaaacacattttgattttttg

SEQ ID NO: 351 - DNA, 22 bp, synthetic construct
  source 1..22: mol_type = other DNA
aaacctcgaaaaggcatgggga

SEQ ID NO: 352 - DNA, 22 bp, synthetic construct
  source 1..22: mol_type = other DNA
tagggctttcccccactgtctg

SEQ ID NO: 353 - DNA, 22 bp, synthetic construct
  source 1..22: mol_type = other DNA
aagccaactcagaaaatccaga

SEQ ID NO: 354 - DNA, 22 bp, synthetic construct
  source 1..22: mol_type = other DNA
actgcatggcctgaggatgagt

SEQ ID NO: 355 - DNA, 22 bp, synthetic construct
  source 1..22: mol_type = other DNA
ctggatgctggatcttccaaat

SEQ ID NO: 356 - DNA, 22 bp, synthetic construct
  source 1..22: mol_type = other DNA
aggtttggaagaccaacctccc

SEQ ID NO: 357 - DNA, 22 bp, synthetic construct
  source 1..22: mol_type = other DNA
tctaacaacagtagtttccgga

SEQ ID NO: 358 - DNA, 22 bp, synthetic construct
  source 1..22: mol_type = other DNA
ttccttctattcgagatctcct

SEQ ID NO: 359 - DNA, 22 bp, synthetic construct
  source 1..22: mol_type = other DNA
ttgacatactttccaatcaata

SEQ ID NO: 360 - DNA, 22 bp, synthetic construct
  source 1..22: mol_type = other DNA
agcctccaagctgtgccttggg

SEQ ID NO: 361 - DNA, 22 bp, synthetic construct
  source 1..22: mol_type = other DNA
ttctattcgagatctcctcgac

SEQ ID NO: 362 - DNA, 22 bp, synthetic construct
  source 1..22: mol_type = other DNA
ggtgggcgttcacggtggtctc

SEQ ID NO: 363 - DNA, 22 bp, synthetic construct
  source 1..22: mol_type = other DNA
aggatcatcaacaaccagcacc

SEQ ID NO: 364 - DNA, 22 bp, synthetic construct
  source 1..22: mol_type = other DNA
ttgagcaggagttgtgcaggtt

SEQ ID NO: 365 - DNA, 22 bp, synthetic construct
  source 1..22: mol_type = other DNA
agatctcctcgacaccgcctct

SEQ ID NO: 366 - DNA, 22 bp, synthetic construct
  source 1..22: mol_type = other DNA
agatcccagagtcagggccctg

SEQ ID NO: 367 - DNA, 22 bp, synthetic construct
  source 1..22: mol_type = other DNA
ataagattgacgatatggcaga

SEQ ID NO: 368 - DNA, 22 bp, synthetic construct
  source 1..22: mol_type = other DNA
agacgagacattatttacacac

SEQ ID NO: 369 - DNA, 22 bp, synthetic construct
  source 1..22: mol_type = other DNA
agaacagtttctctcccaaaag

SEQ ID NO: 370 - DNA, 22 bp, synthetic construct
  source 1..22: mol_type = other DNA
ggccagggttcacccctcccca

SEQ ID NO: 371 - DNA, 22 bp, synthetic construct
  source 1..22: mol_type = other DNA
agggggaacacccgtgtgtctt

SEQ ID NO: 372 - DNA, 22 bp, synthetic construct
  source 1..22: mol_type = other DNA
ggaacagtaaaccctgttccga

SEQ ID NO: 373 - DNA, 22 bp, synthetic construct
  source 1..22: mol_type = other DNA
taggacccctgctcgtgttaca

SEQ ID NO: 374 - DNA, 22 bp, synthetic construct
  source 1..22: mol_type = other DNA
cttccaaaagtaagacaggaaa

SEQ ID NO: 375 - DNA, 22 bp, synthetic construct
  source 1..22: mol_type = other DNA
ggccagggttcaccccaccaca

SEQ ID NO: 376 - DNA, 22 bp, synthetic construct
  source 1..22: mol_type = other DNA
ggataataaggtttaatgcctt

SEQ ID NO: 377 - DNA, 22 bp, synthetic construct
  source 1..22: mol_type = other DNA
tagggctttcccccactgtttg

SEQ ID NO: 378 - DNA, 22 bp, synthetic construct
  source 1..22: mol_type = other DNA
tgagtatttggtgtcttttgga

SEQ ID NO: 379 - DNA, 22 bp, synthetic construct
  source 1..22: mol_type = other DNA
tccccatgccttgtcgaggttt

SEQ ID NO: 380 - DNA, 22 bp, synthetic construct
  source 1..22: mol_type = other DNA
tatgggagtgggcctcagtccg

SEQ ID NO: 381 - DNA, 22 bp, synthetic construct
  source 1..22: mol_type = other DNA
tcatctgccgttccggccgacc

SEQ ID NO: 382 - DNA, 22 bp, synthetic construct
  source 1..22: mol_type = other DNA
ctttctcgccaacttacaaggc

SEQ ID NO: 383 - DNA, 22 bp, synthetic construct
  source 1..22: mol_type = other DNA
ctggatgctgggtcttccaaat

SEQ ID NO: 384 - DNA, 22 bp, synthetic construct
  source 1..22: mol_type = other DNA
gatattgtttacacagaaaggc

SEQ ID NO: 385 - DNA, 22 bp, synthetic construct
  source 1..22: mol_type = other DNA
tctactgtacctgtctttaatc

SEQ ID NO: 386 - DNA, 22 bp, synthetic construct
  source 1..22: mol_type = other DNA
tcctgctgctatgcctcatctt

SEQ ID NO: 387 - DNA, 22 bp, synthetic construct
  source 1..22: mol_type = other DNA
gataagtttcgctccagaccgg

SEQ ID NO: 388 - DNA, 22 bp, synthetic construct
  source 1..22: mol_type = other DNA
gtccgaaggttttgtacagcaa

SEQ ID NO: 389 - DNA, 22 bp, synthetic construct
  source 1..22: mol_type = other DNA
gagccaactcaaacaatccaga

SEQ ID NO: 390 - DNA, 22 bp, synthetic construct
  source 1..22: mol_type = other DNA
tcttcatcctgctgctatgcct

SEQ ID NO: 391 - DNA, 22 bp, synthetic construct
  source 1..22: mol_type = other DNA
gtgcagggtccccagtcctcga

SEQ ID NO: 392 - DNA, 22 bp, synthetic construct
  source 1..22: mol_type = other DNA
gttcccaatcctctgggattct

SEQ ID NO: 393 - DNA, 22 bp, synthetic construct
  source 1..22: mol_type = other DNA
tctggactaccaaggtatgttg

SEQ ID NO: 394 - DNA, 22 bp, synthetic construct
  source 1..22: mol_type = other DNA
gagagaggacaacagagttgtc

SEQ ID NO: 395 - DNA, 22 bp, synthetic construct
  source 1..22: mol_type = other DNA
gttatatggatgatgtggtatt

SEQ ID NO: 396 - DNA, 22 bp, synthetic construct
  source 1..22: mol_type = other DNA
ggccgaccacggggcgcacctc

SEQ ID NO: 397 - DNA, 22 bp, synthetic construct
  source 1..22: mol_type = other DNA
gactactgcctcacccatatcg

SEQ ID NO: 398 - DNA, 22 bp, synthetic construct
  source 1..22: mol_type = other DNA
gcgccgacgggacgtagacaaa

SEQ ID NO: 399 - DNA, 22 bp, synthetic construct
  source 1..22: mol_type = other DNA
gacggaaactgcacttgtattc

SEQ ID NO: 400 - DNA, 22 bp, synthetic construct
  source 1..22: mol_type = other DNA
gtgtaaacaatatctgaacctt

SEQ ID NO: 401 - DNA, 22 bp, synthetic construct
  source 1..22: mol_type = other DNA
ggaactggagccaccagcagga

SEQ ID NO: 402 - DNA, 22 bp, synthetic construct
  source 1..22: mol_type = other DNA
tttgtctttgggtatacatttg

SEQ ID NO: 403 - DNA, 22 bp, synthetic construct
  source 1..22: mol_type = other DNA
ctcttgttgctgtacaaaacct

SEQ ID NO: 404 - DNA, 22 bp, synthetic construct
  source 1..22: mol_type = other DNA
caatcctctgggattctttccc

SEQ ID NO: 405 - DNA, 22 bp, synthetic construct
  source 1..22: mol_type = other DNA
caagattcctatgggagtgggc

SEQ ID NO: 406 - DNA, 22 bp, synthetic construct
  source 1..22: mol_type = other DNA
caagaatatggtgacccacaaa

SEQ ID NO: 407 - DNA, 22 bp, synthetic construct
  source 1..22: mol_type = other DNA
tgctcaaggaacctctatgttt

SEQ ID NO: 408 - DNA, 22 bp, synthetic construct
  source 1..22: mol_type = other DNA
ttatacgggtcaatgtccatgc

SEQ ID NO: 409 - DNA, 22 bp, synthetic construct
  source 1..22: mol_type = other DNA
ttcccgatcaccagttggaccc

SEQ ID NO: 410 - DNA, 22 bp, synthetic construct
  source 1..22: mol_type = other DNA
atcctaaccttaccaaatattt

SEQ ID NO: 411 - DNA, 22 bp, synthetic construct
  source 1..22: mol_type = other DNA
atataagagagaaactacacgc

SEQ ID NO: 412 - DNA, 22 bp, synthetic construct
  source 1..22: mol_type = other DNA
agggggagcacccacgtgtcct

SEQ ID NO: 413 - DNA, 22 bp, synthetic construct
  source 1..22: mol_type = other DNA
ttgagcaggaatcgtgcaggtc

SEQ ID NO: 414 - DNA, 22 bp, synthetic construct
  source 1..22: mol_type = other DNA
actgcatggcctgaggatgact

SEQ ID NO: 415 - DNA, 22 bp, synthetic construct
  source 1..22: mol_type = other DNA
acgcatgcgccgatggcctatg

SEQ ID NO: 416 - DNA, 22 bp, synthetic construct
  source 1..22: mol_type = other DNA
accccaacaaggatcactggcc

SEQ ID NO: 417 - DNA, 22 bp, synthetic construct
  source 1..22: mol_type = other DNA
accaagctctgctagatcccag

SEQ ID NO: 418 - DNA, 22 bp, synthetic construct
  source 1..22: mol_type = other DNA
acagagtatgtaaataatgcct

SEQ ID NO: 419 - DNA, 22 bp, synthetic construct
  source 1..22: mol_type = other DNA
aattacatatcccatgaagtta

SEQ ID NO: 420 - DNA, 22 bp, synthetic construct
  source 1..22: mol_type = other DNA
aatgtatacccaaagacaaaag

SEQ ID NO: 421 - DNA, 22 bp, synthetic construct
  source 1..22: mol_type = other DNA
aatcaataggtctatttacagg

SEQ ID NO: 422 - DNA, 22 bp, synthetic construct
  source 1..22: mol_type = other DNA
tttacaggaggacattattaat

SEQ ID NO: 423 - DNA, 22 bp, synthetic construct
  source 1..22: mol_type = other DNA
aagactgtttgtttaaagactg

SEQ ID NO: 424 - DNA, 22 bp, synthetic construct
  source 1..22: mol_type = other DNA
aaactaggcattatttacatac

SEQ ID NO: 425 - DNA, 22 bp, synthetic construct
  source 1..22: mol_type = other DNA
aaacctcgacaaggcatgggga

SEQ ID NO: 426 - DNA, 22 bp, synthetic construct
  source 1..22: mol_type = other DNA
aaaagtaagacaggaaatgtga

SEQ ID NO: 427 - DNA, 22 bp, synthetic construct
  source 1..22: mol_type = other DNA
aaaactgcctgtaaatagacct

SEQ ID NO: 428 - DNA, 22 bp, synthetic construct
  source 1..22: mol_type = other DNA
aaaacattgcttgatttttagt

SEQ ID NO: 429 - DNA, 22 bp, synthetic construct
  source 1..22: mol_type = other DNA
cacccaggtggccagattcatc

SEQ ID NO: 430 - DNA, 22 bp, synthetic construct
  source 1..22: mol_type = other DNA
ttaactgtaagagggcccacat

SEQ ID NO: 431 - DNA, 22 bp, synthetic construct
  source 1..22: mol_type = other DNA
atggctgctagggtgtgctgcc

SEQ ID NO: 432 - DNA, 22 bp, synthetic construct
  source 1..22: mol_type = other DNA
cactcctcccgcttacagacca

SEQ ID NO: 433 - DNA, 22 bp, synthetic construct
  source 1..22: mol_type = other DNA
ctcttatatagaatgccagcct

SEQ ID NO: 434 - DNA, 22 bp, synthetic construct
  source 1..22: mol_type = other DNA
tgctggtggctccagttccgga

SEQ ID NO: 435 - DNA, 22 bp, synthetic construct
  source 1..22: mol_type = other DNA
ctccagaccggctgcgagcaaa

SEQ ID NO: 436 - DNA, 22 bp, synthetic construct
  source 1..22: mol_type = other DNA
tggaagtagaggacaaacgggc

SEQ ID NO: 437 - DNA, 22 bp, synthetic construct
  source 1..22: mol_type = other DNA
ctcaattttctagggggagcac

SEQ ID NO: 438 - DNA, 22 bp, synthetic construct
  source 1..22: mol_type = other DNA
tgggaacaagagctacagcatg

SEQ ID NO: 439 - DNA, 22 bp, synthetic construct
  source 1..22: mol_type = other DNA
tgggatatgtaattggaagttg

SEQ ID NO: 440 - DNA, 22 bp, synthetic construct
  source 1..22: mol_type = other DNA
cgatcaccagttggaccctgcg

SEQ ID NO: 441 - DNA, 22 bp, synthetic construct
  source 1..22: mol_type = other DNA
cgaggactggggaccctgcacc

SEQ ID NO: 442 - DNA, 22 bp, synthetic construct
  source 1..22: mol_type = other DNA
cctggctcagtttactagtgcc

SEQ ID NO: 443 - DNA, 22 bp, synthetic construct
  source 1..22: mol_type = other DNA
caccttatgagtccaagggata

SEQ ID NO: 444 - DNA, 22 bp, synthetic construct
  source 1..22: mol_type = other DNA
cctctgcctaatcatctcatgt

SEQ ID NO: 445 - DNA, 22 bp, synthetic construct
  source 1..22: mol_type = other DNA
tgtcaacaagaaaaaccccgcc

SEQ ID NO: 446 - DNA, 22 bp, synthetic construct
  source 1..22: mol_type = other DNA
cctcaccatacagcactcaggc

SEQ ID NO: 447 - DNA, 22 bp, synthetic construct
  source 1..22: mol_type = other DNA
tgtcttacttttggaagagaaa

SEQ ID NO: 448 - DNA, 22 bp, synthetic construct
  source 1..22: mol_type = other DNA
tcacattcatttacaggaggac

SEQ ID NO: 449 - DNA, 22 bp, synthetic construct
  source 1..22: mol_type = other DNA
tgttggttcttctggactacca

SEQ ID NO: 450 - DNA, 22 bp, synthetic construct
  source 1..22: mol_type = other DNA
cagtccccaacctccaatcact

SEQ ID NO: 451 - DNA, 22 bp, synthetic construct
  source 1..22: mol_type = other DNA
catcccatcatcctgggctttc

SEQ ID NO: 452 - DNA, 22 bp, synthetic construct
  source 1..22: mol_type = other DNA
tgtggtaaagtaccccaacttc

SEQ ID NO: 453 - DNA, 22 bp, synthetic construct
  source 1..22: mol_type = other DNA
tgttgacaagaatcctcacaat

SEQ ID NO: 454 - DNA, 22 bp, synthetic construct
  source 1..22: mol_type = other DNA
ccatgttcggtgcagggtcccc

SEQ ID NO: 455 - DNA, 22 bp, synthetic construct
  source 1..22: mol_type = other DNA
cccactgtttggctttcagtta

SEQ ID NO: 456 - DNA, 22 bp, synthetic construct
  source 1..22: mol_type = other DNA
catttcctgtcttacttttgga

SEQ ID NO: 457 - DNA, 22 bp, synthetic construct
  source 1..22: mol_type = other DNA
tctggattatcaaggtatgttg

SEQ ID NO: 458 - DNA, 22 bp, synthetic construct
  source 1..22: mol_type = other DNA
tttgtctctgggtatacattta

SEQ ID NO: 459 - DNA, 22 bp, synthetic construct
  source 1..22: mol_type = other DNA
tttgtctaagggcaaatattta

SEQ ID NO: 460 - DNA, 22 bp, synthetic construct
  source 1..22: mol_type = other DNA
tccccatgcctttgcgaggttt

SEQ ID NO: 461 - DNA, 22 bp, synthetic construct
  source 1..22: mol_type = other DNA
ttataagggtcaatgtccatgc

SEQ ID NO: 462 - DNA, 22 bp, synthetic construct
  source 1..22: mol_type = other DNA
tttcctaagattcatttacaag

SEQ ID NO: 463 - DNA, 22 bp, synthetic construct
  source 1..22: mol_type = other DNA
tttacagtgagagggcccacaa

SEQ ID NO: 464 - DNA, 22 bp, synthetic construct
  source 1..22: mol_type = other DNA
tgggttacataattggaagttg

SEQ ID NO: 465 - DNA, 22 bp, synthetic construct
  source 1..22: mol_type = other DNA
ttcccgatcatcagttggaccc

SEQ ID NO: 466 - DNA, 22 bp, synthetic construct
  source 1..22: mol_type = other DNA
tttacaagaggacattattaat

SEQ ID NO: 467 - DNA, 22 bp, synthetic construct
  source 1..22: mol_type = other DNA
tcctcctgcctccaccaatcgg

SEQ ID NO: 468 - DNA, 22 bp, synthetic construct
  source 1..22: mol_type = other DNA
ttggactcataaggtgggaaac

SEQ ID NO: 469 - DNA, 22 bp, synthetic construct
  source 1..22: mol_type = other DNA
tctacagtacctatctttaatc

SEQ ID NO: 470 - DNA, 22 bp, synthetic construct
  source 1..22: mol_type = other DNA
ttccgtcagagatctcctagac

SEQ ID NO: 471 - DNA, 22 bp, synthetic construct
  source 1..22: mol_type = other DNA
ttccttccgtcagagatctcct

SEQ ID NO: 472 - DNA, 22 bp, synthetic construct
  source 1..22: mol_type = other DNA
tgttaacaggcctattgattgg

SEQ ID NO: 473 - DNA, 22 bp, synthetic construct
  source 1..22: mol_type = other DNA
tgtactgtttacttagaaaggc

SEQ ID NO: 474 - DNA, 22 bp, synthetic construct
  source 1..22: mol_type = other DNA
aaaacagtgtttgatcttttgt

SEQ ID NO: 475 - DNA, 22 bp, synthetic construct
  source 1..22: mol_type = other DNA
ccgtctgccgttccagccgacc

SEQ ID NO: 476 - DNA, 22 bp, synthetic construct
  source 1..22: mol_type = other DNA
aagactgtgtgtttaaggactg

SEQ ID NO: 477 - DNA, 22 bp, synthetic construct
  source 1..22: mol_type = other DNA
cctctgcctaatcatctcttgt

SEQ ID NO: 478 - DNA, 22 bp, synthetic construct
  source 1..22: mol_type = other DNA
cctctgcacgttgcatggagac

SEQ ID NO: 479 - DNA, 22 bp, synthetic construct
  source 1..22: mol_type = other DNA
tcacagggtccccagtcctcgc

SEQ ID NO: 480 - DNA, 22 bp, synthetic construct
  source 1..22: mol_type = other DNA
cccctccacacggcggtgtttt

SEQ ID NO: 481 - DNA, 22 bp, synthetic construct
  source 1..22: mol_type = other DNA
ccccagcagagaatggcttgcc

SEQ ID NO: 482 - DNA, 22 bp, synthetic construct
  source 1..22: mol_type = other DNA
cccactgtttggctttcagcta

SEQ ID NO: 483 - DNA, 22 bp, synthetic construct
  source 1..22: mol_type = other DNA
ccatgttcgtcacagggtcccc

SEQ ID NO: 484 - DNA, 22 bp, synthetic construct
  source 1..22: mol_type = other DNA
ccaacttccaattatgtaaccc

SEQ ID NO: 485 - DNA, 22 bp, synthetic construct
  source 1..22: mol_type = other DNA
catctgccggtccgtgtgcact

SEQ ID NO: 486 - DNA, 22 bp, synthetic construct
  source 1..22: mol_type = other DNA
catcccatcgtcctgggctttc

SEQ ID NO: 487 - DNA, 22 bp, synthetic construct
  source 1..22: mol_type = other DNA
cactcctccagcctatagacca

SEQ ID NO: 488 - DNA, 22 bp, synthetic construct
  source 1..22: mol_type = other DNA
caccttatgagtccaaggaata

SEQ ID NO: 489 - DNA, 22 bp, synthetic construct
  source 1..22: mol_type = other DNA
caaccctctgggattctttccc

SEQ ID NO: 490 - DNA, 22 bp, synthetic construct
  source 1..22: mol_type = other DNA
atggctgctaggctgtactgcc

SEQ ID NO: 491 - DNA, 22 bp, synthetic construct
  source 1..22: mol_type = other DNA
atcttctcttttcatttacagt

SEQ ID NO: 492 - DNA, 22 bp, synthetic construct
  source 1..22: mol_type = other DNA
atcctacccacactaaatattt

SEQ ID NO: 493 - DNA, 22 bp, synthetic construct
  source 1..22: mol_type = other DNA
atccgtaggttttgtacagcaa

SEQ ID NO: 494 - DNA, 22 bp, synthetic construct
  source 1..22: mol_type = other DNA
atataagagggaaaccacacgt

SEQ ID NO: 495 - DNA, 22 bp, synthetic construct
  source 1..22: mol_type = other DNA
agtacagtctctcttccaaaag

SEQ ID NO: 496 - DNA, 22 bp, synthetic construct
  source 1..22: mol_type = other DNA
agggggatcacccgtgtgtctt

SEQ ID NO: 497 - DNA, 22 bp, synthetic construct
  source 1..22: mol_type = other DNA
aggatcaacaacaaccagtacg

SEQ ID NO: 498 - DNA, 22 bp, synthetic construct
  source 1..22: mol_type = other DNA
agccgaccacggggcgcacctc

SEQ ID NO: 499 - DNA, 22 bp, synthetic construct
  source 1..22: mol_type = other DNA
aagtaaacagtacatgaacctt

SEQ ID NO: 500 - DNA, 22 bp, synthetic construct
  source 1..22: mol_type = other DNA
actgccttccaccaagctctgc

SEQ ID NO: 501 - DNA, 22 bp, synthetic construct
  source 1..22: mol_type = other DNA
accccatcaaggaccactggcc

SEQ ID NO: 502 - DNA, 22 bp, synthetic construct
  source 1..22: mol_type = other DNA
accaagctctgcaggatcccag

SEQ ID NO: 503 - DNA, 22 bp, synthetic construct
  source 1..22: mol_type = other DNA
cgatacagagctgaggcggtgt

SEQ ID NO: 504 - DNA, 22 bp, synthetic construct
  source 1..22: mol_type = other DNA
aatcaataggcctgttaacagg

SEQ ID NO: 505 - DNA, 22 bp, synthetic construct
  source 1..22: mol_type = other DNA
aaattattacccacccaggtag

SEQ ID NO: 506 - DNA, 22 bp, synthetic construct
  source 1..22: mol_type = other DNA
cgggacgtcctttgtttacgtc

SEQ ID NO: 507 - DNA, 22 bp, synthetic construct
  source 1..22: mol_type = other DNA
tattggttcttctggattatca

SEQ ID NO: 508 - DNA, 22 bp, synthetic construct
  source 1..22: mol_type = other DNA
tatatcttgccttacttttgga

SEQ ID NO: 509 - DNA, 22 bp, synthetic construct
  source 1..22: mol_type = other DNA
taagattcatttacaagaggac

SEQ ID NO: 510 - DNA, 22 bp, synthetic construct
  source 1..22: mol_type = other DNA
gttcccaaccctctgggattct

SEQ ID NO: 511 - DNA, 22 bp, synthetic construct
  source 1..22: mol_type = other DNA
gtcgtcctctcgcggaaatata

SEQ ID NO: 512 - DNA, 22 bp, synthetic construct
  source 1..22: mol_type = other DNA
gtcagagatctcctagacaccg

SEQ ID NO: 513 - DNA, 22 bp, synthetic construct
  source 1..22: mol_type = other DNA
aaaagtaaggcaagatatatga

SEQ ID NO: 514 - DNA, 22 bp, synthetic construct
  source 1..22: mol_type = other DNA
ggccagggttcacccctccaca

SEQ ID NO: 515 - DNA, 22 bp, synthetic construct
  source 1..22: mol_type = other DNA
ggattaaagataggtactgtag

SEQ ID NO: 516 - DNA, 22 bp, synthetic construct
  source 1..22: mol_type = other DNA
ggaacagtaaaccctgctccga

SEQ ID NO: 517 - DNA, 22 bp, synthetic construct
  source 1..22: mol_type = other DNA
ggaaactactgttgttagacga

SEQ ID NO: 518 - DNA, 22 bp, synthetic construct
  source 1..22: mol_type = other DNA
gctatatggatgatgtggtatt

SEQ ID NO: 519 - DNA, 22 bp, synthetic construct
  source 1..22: mol_type = other DNA
gcgagaggacgacagaattgtc

SEQ ID NO: 520 - DNA, 22 bp, synthetic construct
  source 1..22: mol_type = other DNA
gcgacgcggcgattgagatctg

SEQ ID NO: 521 - DNA, 22 bp, synthetic construct
  source 1..22: mol_type = other DNA
gatgagctttgctccagaccgg

SEQ ID NO: 522 - DNA, 22 bp, synthetic construct
  source 1..22: mol_type = other DNA
gagtgtggattcgcactcctcc

SEQ ID NO: 523 - DNA, 22 bp, synthetic construct
  source 1..22: mol_type = other DNA
gagcagggtttactgttcctga

SEQ ID NO: 524 - DNA, 22 bp, synthetic construct
  source 1..22: mol_type = other DNA
gacttctttccttccgtcagag

SEQ ID NO: 525 - DNA, 22 bp, synthetic construct
  source 1..22: mol_type = other DNA
aaaccagacattatttacatac

SEQ ID NO: 526 - DNA, 22 bp, synthetic construct
  source 1..22: mol_type = other DNA
aaagagtatgtaaataatgtct

SEQ ID NO: 527 - DNA, 22 bp, synthetic construct
  source 1..22: mol_type = other DNA
cttttcatttacagtgagaggg

SEQ ID NO: 528 - DNA, 22 bp, synthetic construct
  source 1..22: mol_type = other DNA
cttcacctctgcacgttgcatg

SEQ ID NO: 529 - DNA, 22 bp, synthetic construct
  source 1..22: mol_type = other DNA
ctgctggggggaattgatgact

SEQ ID NO: 530 - DNA, 22 bp, synthetic construct
  source 1..22: mol_type = other DNA
ctcttatatagaataccagcct

SEQ ID NO: 531 - DNA, 22 bp, synthetic construct
  source 1..22: mol_type = other DNA
ctcaattttctagggggatcac

SEQ ID NO: 532 - DNA, 22 bp, synthetic construct
  source 1..22: mol_type = other DNA
ctaaacttcatgggttacataa

SEQ ID NO: 533 - DNA, 22 bp, synthetic construct
  source 1..22: mol_type = other DNA
cggtggtctccatgcaacgtgc

SEQ ID NO: 534 - DNA, 22 bp, synthetic construct
  source 1..22: mol_type = other DNA
cgatcatcagttggaccctgca

SEQ ID NO: 535 - DNA, 22 bp, synthetic construct
  source 1..22: mol_type = other DNA
aattatgtaacccatgaagttt

SEQ ID NO: 536 - DNA, 22 bp, synthetic construct
  source 1..22: mol_type = other DNA
aagactgtgtgtttaatgactg

SEQ ID NO: 537 - DNA, 22 bp, synthetic construct
  source 1..22: mol_type = other DNA
tttgcaggaggacattattaat

SEQ ID NO: 538 - DNA, 22 bp, synthetic construct
  source 1..22: mol_type = other DNA
tcacattcatttgcaggaggac

SEQ ID NO: 539 - DNA, 22 bp, synthetic construct
  source 1..22: mol_type = other DNA
ttgagcaggagtcgtgcaggtc

SEQ ID NO: 540 - DNA, 22 bp, synthetic construct
  source 1..22: mol_type = other DNA
cactcctcctgcttacagacca

SEQ ID NO: 541 - DNA, 22 bp, synthetic construct
  source 1..22: mol_type = other DNA
tgtaaatagacctattgattgg

SEQ ID NO: 542 - DNA, 22 bp, synthetic construct
  source 1..22: mol_type = other DNA
cgatcaccagttggaccctgca

SEQ ID NO: 543 - DNA, 22 bp, synthetic construct
  source 1..22: mol_type = other DNA
ctcttatataaaatgcccgcct

SEQ ID NO: 544 - DNA, 22 bp, synthetic construct
  source 1..22: mol_type = other DNA
gaaacttcctgtaaatagacct

SEQ ID NO: 545 - DNA, 22 bp, synthetic construct
  source 1..22: mol_type = other DNA
gactactgcctctcccatatcg

SEQ ID NO: 546 - DNA, 22 bp, synthetic construct
  source 1..22: mol_type = other DNA
tcgtctgccgttccggccgacc

SEQ ID NO: 547 - DNA, 22 bp, synthetic construct
  source 1..22: mol_type = other DNA
ttgtactaggaggctgtaggca

SEQ ID NO: 548 - DNA, 22 bp, synthetic construct
  source 1..22: mol_type = other DNA
gaaaacattgtttgattttttg

SEQ ID NO: 549 - DNA, 22 bp, synthetic construct
  source 1..22: mol_type = other DNA
cctctgcacgtcgcatggagac

(19) United States
(12) Patent Application Publication
(10) Pub. No.: US 2025/0257340 A1
(43) Pub. Date: Aug. 14, 2025

(54) GUIDE NUCLEIC ACID IDENTIFICATION AND METHODS OF USE

(71) Applicant: Excision BioTherapeutics Inc, San Francisco, CA (US)

(72) Inventors: Thomas James CRADICK, Belmont, MA (US); Ethan Yixun XU, Winchester, MA (US); Xinting YAO, Revere, MA (US)

(21) Appl. No.: 18/172,946

(22) Filed: Feb. 22, 2023

Related U.S. Application Data
(60) Provisional application No. 63/313,033, filed on Feb. 23, 2022.
(60) Provisional application No. 63/313,059, filed on Feb. 23, 2022.
(60) Provisional application No. 63/313,037, filed on Feb. 23, 2022.

(51) Int. Cl. C12N 9/22; C12N 15/11

(52) U.S. Cl. CPC C12N 9/22; C12N 15/11; C12N 2310/20; C12N 2800/80

(57) ABSTRACT

Provided herein are gene editing compositions, systems, vectors, and methods that effectively modulate and/or edit a Hepatitis B virus genome.

### CROSS-REFERENCE

This application claims the benefit of U.S. Provisional Application No. 63/313,033, filed Feb. 23, 2022; U.S. Provisional Application No. 63/313,059, filed Feb. 23, 2022; and U.S. Provisional Application No. 63/313,037, filed Feb. 23, 2022, which applications are incorporated herein by reference.

### SEQUENCE LISTING

The instant application contains a sequence listing which has been submitted electronically in xml format and is hereby incorporated by reference in its entirety. The xml copy, created on Feb. 20, 2022, is named 56852785201_SL.xml and is 501,136 bytes in size.

### BACKGROUND

Hepatitis B virus (HBV) is a cause of liver diseases and disorders including acute hepatitis that can lead to fulminate hepatic failure as well as chronic hepatitis, cirrhosis, and heptocellular carcinoma (HCC). There are several challenges associated with hepatitis B virus (HBV) therapy, including: Viral persistence (HBV can persist in the liver and continue to cause damage even after antiviral therapy), drug resistance (some patients may develop resistance to antiviral drugs, which can limit the effectiveness of treatment), and overall limited treatment. Accordingly, HBV can infection millions of people worldwide. Improved therapies are needed for targeting HBV.

### SUMMARY

Described herein, in certain embodiments, are compositions comprising: (a) a Clustered Regularly Interspaced Short Palindromic Repeat (CRISPR)-associated endonuclease or a nucleic acid sequence encoding the CRISPR-associated endonuclease; and (b) one or more guide RNAs (gRNAs) or a nucleic acid sequence encoding the one or more gRNAs, the one or more gRNA hybridizes or is complementary to a target nucleic acid sequence within a Hepatitis B Virus (HBV) genome, the HBV genome comprising at least about 90% sequence identity to any one of SEQ ID NOs: 2-5. In some embodiments, the HBV genome comprises a sequence of any one of SEQ ID NOs: 2-5. In some embodiments, a gRNA of the one or more gRNAs is encoded by a sequence according to any one of SEQ ID NOs: 6-272 or a sequence according to any one of SEQ ID NOs: 6-272 comprising 1, 2, or 3 modifications. In some embodiments, the modification is a substitution, deletion, insertion, or a combination thereof. In some embodiments, a gRNA comprises a region that hybridizes to a target nucleic acid sequence within a HBV genome, the HBV genome comprising at least about 90% sequence identity to any one of SEQ ID NOs: 2-5. In some embodiments, the target nucleic acid sequence is located within a structural gene, non-structural gene, or combinations thereof. In some embodiments, the target nucleic acid sequence is located within a C, X, P, or S region. In some embodiments, the CRISPR-associated endonuclease is Type I, Type II, or Type III Cas endonuclease. In some embodiments, the CRISPR-associated endonuclease is a Cas9 endonuclease, a Cas12 endonuclease, a CasX endonuclease, or a CasΦ endonuclease. In some embodiments, the CRISPR-associated endonuclease is a Cas9 endonuclease. In some embodiments, the Cas9 endonuclease is a Staphylococcus aureus Cas9 endonuclease. In some embodiments, the HBV is HBV-A genotype. In some embodiments, the HBV is HBV-B genotype. In some embodiments, the HBV is HBV-C genotype. In some embodiments, the HBV is HBV-A, HBV-B, HBV-C, HBV-D, HBV-E, HBV-F, HBV-G, or HBV-H genotype. In some embodiments, the HBV is HBV-A1, HBV-A2, HBV-QS-A3, or HBV-A4 genotype. In some embodiments, the HBV is HBV-B1, HBV-B2, HBV-QS-B3, HBV-B4, or HBV-B5 genotype. In some embodiments, the HBV is HBV-C1, HBV-QS-C2, HBV-C3, HBV-C4, HBV-C5, or HBV-C6-C15 genotype. In some embodiments, the HBV is HBV-D1, HBV-D2, HBV-D3, HBV-D4, HBV-D5, or HBV-D6 genotype. In some embodiments, the HBV is HBV-F1, HBV-F2, HBV-F3, or HBV-F4 genotype.

Described herein, in certain embodiments, are CRISPR-Cas systems comprising: (a) a Clustered Regularly Interspaced Short Palindromic Repeat (CRISPR)-associated endonuclease; and (b) one or more guide RNAs (gRNAs) or a nucleic acid sequence encoding the one or more gRNAs, the one or more gRNA hybridizes or is complementary to a target nucleic acid sequence within a Hepatitis B Virus (HBV) genome, the HBV genome comprising at least about 90% sequence identity to any one of SEQ ID NOs: 2-5.

Described herein, in certain embodiments, are nucleic acids encoding the CRISPR-Cas systems described herein.

Described herein, in certain embodiments, are vectors comprising a nucleic acid encoding: (a) a Clustered Regularly Interspaced Short Palindromic Repeat (CRISPR)-associated endonuclease; and (b) one or more guide RNAs (gRNAs) or a nucleic acid sequence encoding the one or more gRNAs, the one or more gRNA hybridizes or is complementary to a target nucleic acid sequence within a Hepatitis B Virus (HBV) genome, the HBV genome comprising at least about 90% sequence identity to any one of SEQ ID NOs: 2-5. In some embodiments, the HBV genome comprises a sequence of any one of SEQ ID NOs: 2-5. In some embodiments, a gRNA of the one or more gRNAs is encoded by a sequence according to any one of SEQ ID NOs: 6-272 or a sequence according to any one of SEQ ID NOs: 6-272 comprising 1, 2, or 3 modifications. In some embodiments, the modification is a substitution, deletion, insertion, or a combination thereof. In some embodiments, a gRNA comprises a region that hybridizes to a target nucleic acid sequence within a HBV genome, the HBV genome comprising at least about 90% sequence identity to any one of SEQ ID NOs: 2-5. In some embodiments, the target nucleic acid sequence is located within a structural gene, non-structural gene, or combinations thereof. In some embodiments, the target nucleic acid sequence is located within a C, X, P, or S region. In some embodiments, the CRISPR-associated endonuclease is Type I, Type II, or Type III Cas endonuclease. In some embodiments, the CRISPR-associated endonuclease is a Cas9 endonuclease, a Cas12 endonuclease, a CasX endonuclease, or a CasΦ endonuclease. In some embodiments, the CRISPR-associated endonuclease is a Cas9 endonuclease. In some embodiments, the Cas9 endonuclease is a Staphylococcus aureus Cas9 endonuclease. In some embodiments, the HBV is HBV-A genotype. In some embodiments, the HBV is HBV-B genotype. In some embodiments, the HBV is HBV-C genotype. In some embodiments, the HBV is HBV-A, HBV-B, HBV-C, HBV-D, HBV-E, HBV-F, HBV-G, or HBV-H genotype. In some embodiments, the HBV is HBV-A1, HBV-A2, HBV-QS-A3, or HBV-A4 genotype. In some embodiments, the HBV is HBV-B1, HBV-B2, HBV-QS-B3, HBV-B4, or HBV-B5 genotype. In some embodiments, the HBV is HBV-C1, HBV-QS-C2, HBV-C3, HBV-C4, HBV-C5, or HBV-C6-C15 genotype. In some embodiments, the HBV is HBV-D1, HBV-D2, HBV-D3, HBV-D4, HBV-D5, or HBV-D6 genotype. In some embodiments, the HBV is HBV-F1, HBV-F2, HBV-F3, or HBV-F4 genotype. In some embodiments, the nucleic acid further comprises a promoter. In some embodiments, the promoter is a ubiquitous promoter. In some embodiments, the promoter is a tissue-specific promoter. In some embodiments, the promoter is a constitutive promoter. In some embodiments, the promoter is a human cytomegalovirus promoter. In some embodiments, the nucleic acid further comprises an enhancer element. In some embodiments, the enhancer element is a human cytomegalovirus enhancer element. In some embodiments, the nucleic acid further comprises a 5′ ITR element and 3′ ITR element. In some embodiments, the vector is an adeno-associated virus (AAV) vector. In some embodiments, the adeno-associated virus (AAV) vector is AAV2, AAV5, AAV6, AAV7, AAV8, or AAV9. In some embodiments, the vector is an AAV6 vector or an AAV9 vector.

Described herein, in certain embodiments, are methods of excising part or all of a Hepatitis B Virus (HBV) sequence from a cell, the method comprising providing to the cell the compositions described herein, the CRISPR-Cas systems described herein, or the vector described herein. Described herein, in certain embodiments, are methods of inhibiting or reducing Hepatitis B Virus (HBV) replication in a cell, the method comprising providing to the cell the compositions described herein, the CRISPR-Cas systems described herein, or the vectors described herein. In some embodiments, the cell is in a subject. In some embodiments, the subject is a human.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows an exemplary flowchart of a method for selecting a guide RNA.

FIG. 2 schematically illustrates a computer system that is programmed or otherwise configured to implement methods provided herein.

### DETAILED DESCRIPTION

Provided herein are gene editing systems (e.g. CRISPR-Cas9, CRISPR-CasX) useful for inhibiting, reducing, or ameliorating Hepatitis B virus (HBV) infection. For instance, provided herein are methods of for inhibiting, reducing, or ameliorating HBV infection by the gene editing systems that effectively edit the HBV genome. Provided herein are programmable nucleases that target the HBV viral genomic DNA that is either integrated into a host cell's genome or maintained extrachromosomally (e.g. not integrated into the genome of a host cell). For example, in some instances, targeting specific genes or elements of the HBV genome leads to the depletion of infectious viral genomes and/or excision of regions within the viral genome at coding sequences or regulatory regions to inactivate viral replication. Provided herein, in certain embodiments, are compositions and methods that result in producing fewer off-target effects by targeting sequences in the HBV genome but not in the host genome.

Programmable nucleases enable precise genome editing by, in some instances, introducing DNA double-strand breaks (DSBs) at specific genomic loci, thereby initiating gene editing. Generally, as embodied herein, a variety of gene editing systems can be employed to target the HBV genes, elements, or regions described herein. In some embodiments, the gene editing system comprises a CRISPR-Cas system. In some embodiments, the gene editing system comprises meganucleases. In some embodiments, the gene editing system comprises zinc finger nucleases (ZFNs). In some embodiments, the gene editing system comprises transcription activator-like effector nucleases (TALENs). These gene editing systems can be broadly classified into two categories based on their mode of DNA recognition: ZFNs, TALENs and meganucleases achieve specific DNA binding via protein-DNA interactions, whereas CRISPR-Cas systems are targeted to specific DNA sequences by a short RNA guide molecule that base-pairs directly with the target DNA and by protein-DNA interactions. Accordingly, protein targeting or nucleic acid targeting can be employed to target the HBV DNA loci described herein.

For example, described and provided herein are CRISPR-Cas compositions and methods useful for reducing or inhibiting HBV replication within a cell. By way of further example, the described and provided CRISPR-Cas compositions are useful for modulating (e.g. altering, removing, etc.) and/or editing (e.g. excising) genomic HBV DNA in a cell, thereby inhibiting HBV replication. Inhibiting HBV by modulating and/or editing HBV DNA in a cell yields an incompetent and/or deficient HBV genome structure that does not allow for replication of the HBV virus. As such, the CRISPR-Cas compositions and methods described herein can be used to effectively modulate and/or edit a HBV genome in a cell. In some embodiments, the cells already comprise a HBV infection. In some embodiments, the CRISPR systems provided herein eliminate or reduce a latently infected cell. In some embodiments, the CRISPR systems provided herein modulate and/or edit HBV DNA in a latently infected cell. In some embodiments, the CRISPR systems provided herein prevent HBV infection.

Provided and described herein are systems (e.g., computer-implemented systems) for selecting, evaluating, or prioritizing a guide RNA candidate comprising: at least one processor, a memory, and instructions executable by the at least one processor comprising: (a) a data storage system comprising a target sequence (e.g., consensus sequence); and (b) one or more modules communicatively coupled to the data storage system, wherein the one or more modules are configured to: (i) identify one or more targeting sites in the target sequence; (ii) identify one or more guide target sequences in proximity to the one or more targeting sites; and (iii) calculate one or more criteria based on the one or more guide target sequences or a guide RNA corresponding to at least one of the one more guide target sequence, wherein the guide RNA candidate is selected based on the one or more criteria of step (b) (iii). In some embodiments, the one or more targeting sites are adjacent or proximal to (e.g., within 10 bp) protospacer adjacent motifs (PAMs).

Provided and described herein are systems (e.g., computer-implemented systems) for selecting, evaluating, or prioritizing a guide RNA candidate comprising: at least one processor, a memory, and instructions executable by the at least one processor comprising: (a) a data storage system comprising a target sequence (e.g., consensus sequence); and (b) one or more modules communicatively coupled to the data storage system, wherein the one or more modules are configured to: (i) identify one or more protospacer adjacent motifs (PAMs) in the target sequence; (ii) identify one or more guide target sequences in proximity to the one or more PAMs; and (iii) calculate one or more criteria based on the one or more guide target sequences or a guide RNA corresponding to at least one of the one more guide target sequence, wherein the guide RNA candidate is selected based on the one or more criteria of step (b) (iii).

Also provided and described herein are systems for selecting, evaluating, or prioritizing a guide RNA candidate comprising: at least one processor, a memory, and instructions executable by the at least one processor comprising: (a) a data storage system comprising a target sequence (e.g., consensus sequence); (b) a targeting site identifier configured to identify one or more targeting sites in the target sequence; (c) a proximity identifier configured to identify one or more guide target sequences in proximity to the one or more targeting sites; and (d) a criteria analysis module configured to calculate one or more criteria based on the one or more guide target sequences or a guide RNA corresponding to at least one of the one or more guide target sequence, wherein the guide RNA candidate is selected based on the one or more criteria of step (d). In some embodiments, the one or more targeting sites are adjacent or proximal to (e.g., within 10 bp) protospacer adjacent motifs (PAMs).

Also provided and described herein are systems for selecting, evaluating, or prioritizing a guide RNA candidate comprising: at least one processor, a memory, and instructions executable by the at least one processor comprising: (a) a data storage system comprising a target sequence (e.g., consensus sequence); (b) a protospacer adjacent motif (PAM) identifier configured to identify one or more PAMs in the target sequence; (c) a proximity identifier configured to identify one or more guide target sequences in proximity to the one or more PAMs; and (d) a criteria analysis module configured to calculate one or more criteria based on the one or more guide target sequences or a guide RNA corresponding to at least one of the one or more guide target sequence, wherein the guide RNA candidate is selected based on the one or more criteria of step (d).

Further provided herein are methods for selecting, evaluating, or prioritizing a guide RNA candidate, comprising: (a) providing a target sequence (e.g., consensus sequence); (b) identifying one or more targeting sites in the target sequence; (c) identifying one or more guide target sequences in proximity to the one or more targeting sites; (d) calculating one or more criteria based on the one more guide target sequences or a guide RNA corresponding to at least one of the one more guide target sequence; and (e) selecting the guide RNA candidate based on the one or more criteria of step (d). In some embodiments, the one or more targeting sites are adjacent or proximal to (e.g., within 10 bp) protospacer adjacent motifs (PAMs).

Further provided herein are methods for selecting, evaluating, or prioritizing a guide RNA candidate, comprising: (a) providing a target sequence (e.g., consensus sequence); (b) identifying one or more PAMs in the target sequence; (c) identifying one or more guide target sequences in proximity to the one or more PAMs; (d) calculating one or more criteria based on the one more guide target sequences or a guide RNA corresponding to at least one of the one more guide target sequence; and (e) selecting the guide RNA candidate based on the one or more criteria of step (d).

Further provided herein are computer-implemented systems to select, evaluate, or prioritize a targeting site, the system comprising: at least one processor, a memory, and instructions executable by the at least one processor comprising: a) a data storage system comprising a target sequence (e.g., consensus sequence) comprising at least about 90% sequence identity to any one of SEQ ID NOs: 2-5; b) a criteria analysis module configured to calculate one or more criteria of the targeting site; and c) a target identifier for selecting the target site based on the one or more criteria of step (b). In some embodiments, the one or more criteria is based on positional entropy, conservation, a knockout score, an overlapping reading frame, a gene location, a coding region, a non-coding region, predicted cutting rate or efficiency, efficacy, a frequency of a targeting site (e.g., a PAM site), or combinations thereof. Provided herein are computer-implemented systems to select, evaluate, or prioritize a targeting site, the system comprising: at least one processor, a memory, and instructions executable by the at least one processor comprising: a) a data storage system comprising a target sequence (e.g., consensus sequence; and b) a criteria analysis module configured to calculate one or more criteria of the targeting site, wherein the one or more criteria is based on positional entropy, conservation, a knockout score, an overlapping reading frame, a gene location, a coding region, a non-coding region, predicted cutting rate or efficiency, efficacy, a frequency of a targeting site (e.g., a PAM site), or combinations thereof; and c) a target identifier for selecting the target site based on the one or more criteria of step (b). In some embodiments, the targeting site is recognized by a zinc finger nuclease, a transcription activator-like effector nuclease (TALEN), a meganuclease, or a CRISPR-associated protein (e.g., Cas).

### Cas Nucleases

Engineered CRISPR systems generally contain two components: a guide RNA (gRNA or sgRNA) and a CRISPR-associated endonuclease (Cas protein). In nature, CRISPR/CRISPR-associated (Cas) systems provide bacteria and archaea with adaptive immunity against viruses and plasmids by using CRISPR RNAs (crRNAs) to guide the silencing of invading nucleic acids. The CRISPR-Cas is a RNA-mediated adaptive defense system that relies on small RNA molecules for sequence-specific detection and silencing of foreign nucleic acids. CRISPR/Cas systems are composed of cas genes organized in operon(s) and CRISPR array(s) consisting of genome-targeting sequences (called spacers). Provided herein are engineered CRISPR systems that detect and silence HBV DNA in a cell.

As described herein, CRISPR-Cas systems generally refer to an enzyme system that includes a guide RNA sequence that contains a nucleotide sequence complementary or substantially complementary to a region of a target polynucleotide (e.g. HBV genomic DNA), and a protein with nuclease activity. CRISPR-Cas systems include Type I CRISPR-Cas system, Type II CRISPR-Cas system, Type III CRISPR-Cas system, and derivatives thereof. CRISPR-Cas systems include engineered and/or programmed nuclease systems derived from naturally accruing CRISPR-Cas systems. In certain embodiments, CRISPR-Cas systems contain engineered and/or mutated Cas proteins. In some embodiments, nucleases generally refer to enzymes capable of cleaving the phosphodiester bonds between the nucleotide subunits of nucleic acids. In some embodiments, endonucleases are generally capable of cleaving the phosphodiester bond within a polynucleotide chain. Nickases refer to endonucleases that cleave only a single strand of a DNA duplex.

In some embodiments, CRISPR-Cas systems further comprise transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. In certain embodiments, a target sequence comprises any polynucleotide, such as DNA or RNA polynucleotides. In some embodiments, a target sequence is located in the nucleus or cytoplasm of a cell.

In some embodiments, the CRISPR/Cas system used herein can be a type I, a type II, or a type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, CasX, CasΦ, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. By way of further example, in some embodiments, the CRISPR-Cas protein is a Cas1, Cas1B, Cas2, Cas3, Cas4, Cas5, Cash, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx16, CsaX, Csx3, Csx1, Csx15, Csf1, Csf2, Csf3, Csf4, Cas9, Cas12 (e.g., Cas12a, Cas12b, Cas12c, Cas12d, Cas12k, Cas12j/CasΦ, Cas12L etc.), Cas13 (e.g., Cas13a, Cas13b (such as Cas13b-t1, Cas13b-t2, Cas13b-t3), Cas13c, Cas13d, etc.), Cas14, CasX, CasY, or an engineered form of the Cas protein. In some embodiments, the CRISPR/Cas protein or endonuclease is Cas9. In some embodiments, the CRISPR/Cas protein or endonuclease is Cas12. In certain embodiments, the Cas12 polypeptide is Cas12a, Cas12b, Cas12c, Cas12d, Cas12e, Cas12g, Cas12h, Cas12i, Cas12L or Cas12J. In some embodiments, the CRISPR/Cas protein or endonuclease is CasX. In some embodiments, the CRISPR/Cas protein or endonuclease is CasY. In some embodiments, the CRISPR/Cas protein or endonuclease is Cas.

In some embodiments, the Cas9 protein can be from or derived from: Staphylococcus aureus, Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Fine goldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

In some embodiments, the composition comprises a CRISPR-associated (Cas) protein, or functional fragment or derivative thereof. In some embodiments, the Cas protein is an endonuclease, including but not limited to the CasX endonuclease. In some embodiments, the CasX protein comprises an amino acid sequence identical to the wild deltaproteobacteria or planctomycetes CasX amino acid sequence. In some embodiments, the Cas protein comprises the amino acid sequence of deltaproteobacteria CasX amino acid sequence. In some embodiments, the Cas protein comprises the amino acid sequence of planctomycetes CasX amino acid sequence.

In some embodiments, the composition comprises a CRISPR-associated (Cas) protein, or functional fragment or derivative thereof. In some embodiments, the Cas protein is an endonuclease, including but not limited to the Cas9 endonuclease. In some embodiments, the Cas9 protein comprises an amino acid sequence identical to the wild type Streptococcus pyogenes or Staphylococcus aureus Cas9 amino acid sequence. In some embodiments, the Cas protein comprises the amino acid sequence of a Cas protein from other species, for example other Streptococcus species, such as thermophilus; Pseudomonas aeruginosa, Escherichia coli, or other sequenced bacteria genomes and archaea, or other prokaryotic microorganisms. Other Cas proteins, useful for the present disclosure, known or can be identified, using methods known in the art (see e.g., Esvelt et al., 2013, Nature Methods, 10:1116-1121). In some embodiments, the Cas protein comprises a modified amino acid sequence, as compared to its natural source.

CRISPR/Cas proteins comprise at least one RNA recognition and/or RNA binding domain. RNA recognition and/or RNA binding domains interact with guide RNAs (gRNAs). CRISPR/Cas proteins can also comprise nuclease domains (i.e., DNase or RNase domains), DNA binding domains, helicase domains, RNAse domains, protein-protein interaction domains, dimerization domains, as well as other domains.

The CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the Cas protein. The CRISPR/Cas-like protein can also be truncated or modified to optimize the activity of the effector domain of the Cas protein.

In some embodiments, the CRISPR/Cas-like protein can be derived from a wild type Cas protein or fragment thereof. In some embodiments, the CRISPR/Cas-like protein is a modified Cas9 protein. For example, the amino acid sequence of the Cas9 protein can be modified to alter one or more properties (e.g., nuclease activity, affinity, stability, etc.) of the protein relative to wild-type or another Cas protein. Alternatively, domains of the Cas9 protein not involved in RNA-guided cleavage can be eliminated from the protein such that the modified Cas9 protein is smaller than the wild-type Cas9 protein.

The disclosed CRISPR-Cas compositions should also be construed to include any form of a protein having substantial homology to a Cas protein (e.g., Cas9, saCas9, Cas9 protein) disclosed herein. In some embodiments, a protein which is “substantially homologous” is about 50% homologous, about 70% homologous, about 80% homologous, about 90% homologous, about 95% homologous, or about 99% homologous to amino acid sequence of a Cas protein disclosed herein.

In some embodiments, the CRISPR/Cas-like protein can be derived from a wild type Cas protein or fragment thereof. In some embodiments, the CRISPR/Cas-like protein is a modified CasX protein. For example, the amino acid sequence of the CasX protein can be modified to alter one or more properties (e.g., nuclease activity, affinity, stability, etc.) of the protein relative to wild-type or another Cas protein. Alternatively, domains of the CasX protein not involved in RNA-guided cleavage can be eliminated from the protein such that the modified CasX protein is smaller than the wild-type CasX protein.

The disclosed CRISPR-Cas compositions should also be construed to include any form of a protein having substantial homology to a Cas protein (e.g., CasX, saCasX, CasX protein) disclosed herein. In some embodiments, a protein which is “substantially homologous” is about 50% homologous, about 70% homologous, about 80% homologous, about 90% homologous, about 95% homologous, or about 99% homologous to amino acid sequence of a Cas protein disclosed herein.

### HBV Targeting

The CRISPR-Cas systems described herein achieve, in some embodiments, the effective modulation and/or editing of a HBV genome through use of gRNA targets. CRISPR-Cas systems, in some embodiments, are designed to target a nucleic acid sequence in a HBV genome.

In some embodiments, the target nucleic acid sequence is located within a structural gene, non-structural gene, or combinations thereof. In some embodiments, the target nucleic acid sequence is located within a C, X, P, or S region.

In some embodiments, the target nucleic acid sequence is a naturally occurring sequence. In other embodiments, the target nucleic acid sequence is a non-naturally occurring sequence.

In some embodiments, the HBV is HBV-A genotype. In some embodiments, the HBV is HBV-B genotype. In some embodiments, HBV-C genotype. In some embodiments, the HBV is HBV-A, HBV-B, HBV-C, HBV-D, HBV-E, HBV-F, HBV-G, or HBV-H genotype. In some embodiments, the HBV is HBV-A1, HBV-A2, HBV-QS-A3, or HBV-A4 genotype. In some embodiments, the HBV is HBV-B1, HBV-B2, HBV-QS-B3, HBV-B4, or HBV-B5 genotype. In some embodiments, the HBV is HBV-C1, HBV-QS-C2, HBV-C3, HBV-C4, HBV-C5, or HBV-C6-C15 genotype. In some embodiments, the HBV is HBV-D1, HBV-D2, HBV-D3, HBV-D4, HBV-D5, or HBV-D6 genotype. In some embodiments, the HBV is HBV-F1, HBV-F2, HBV-F3, or HBV-F4 genotype.

Described herein, in some embodiments, are compositions comprising (a) a Clustered Regularly Interspaced Short Palindromic Repeat (CRISPR)-associated endonuclease or a nucleic acid sequence encoding the CRISPR-associated endonuclease; and (b) one or more guide RNAs (gRNAs) or a nucleic acid sequence encoding the one or more gRNAs, the one or more gRNA hybridizes or is complementary to a target nucleic acid sequence within a Hepatitis B Virus (HBV) genome, the HBV genome comprising at least about 90% sequence identity to any one of SEQ ID NOs: 2-5. In some embodiments, the HBV genome comprises at least about 70%, 75%, 80%, 85%, 90%, or 95% identity to any one of SEQ ID NOs: 2-5. In some embodiments, the HBV genome comprising at least about 80%, 85%, 90%, 91%, 92%, 93%, 93%, 94%, 95%, 96%, 97%, 98%, or 99% identity to any one of SEQ ID NOs: 2-5. In some embodiments, the HBV genome comprises a sequence of any one of SEQ ID NOs: 2-5.

SEQ ID NOs: 2-5 are non-naturally occurring consensus sequences generated by aligning different HBV strains, e.g., strains within the same HBV genotype or strains from different HBV genotypes. For example, SEQ ID NO: 2 is a consensus sequence of Clustal-Omega HBV Genotype A strains; SEQ ID NO: 3 is a consensus sequence of Clustal-Omega HBV Genotype B strains; SEQ ID NO: 4 is a consensus sequence of Clustal-Omega HBV Genotype C strains; and SEQ ID NO: 5 is a consensus sequence of Clustal-Omega HBV Genotypes B and C strains.

TABLE 1
Sequences

| SEQ ID | |
|---|---|
| NO. | Sequence |
| 1 | AATTCCACAACCTTCCACCAAACTCTGCAAGATCCCAGAGTGAGAGGCCTGTATTTCCCTGCT |
| | GGTGGCTCCAGTTCAGGAACAGTAAACCCTGTTCTGACTACTGCCTCTCCCTTATCGTCAATC |
| | TTCTCGAGGATTGGGGACCCTGCGCTGAACATGGAGAACATCACATCAGGATTCCTAGGACCC |
| | CTTCTCGTGTTACAGGCGGGGTTTTTCTTGTTGACAAGAATCCTCACAATACCGCAGAGTCTA |
| | GACTCGTGGTGGACTTCTCTCAATTTTCTAGGGGGAACTACCGTGTGTCTTGGCCAAAATTCG |
| | CAGTCCCCAACCTCCAATCACTCACCAACCTCTTGTCCTCCAACTTGTCCTGGTTATCGCTGG |
| | ATGTGTCTGCGGCGTTTTATCATCTTCCTCTTCATCCTGCTGCTATGCCTCATCTTCTTGTTG |
| | GTTCTTCTGGACTATCAAGGTATGTTGCCCGTTTGTCCTCTAATTCCAGGATCCTCAACAACC |
| | AGCACGGGACCATGCCGGACCTGCATGACTACTGCTCAAGGAACCTCTATGTATCCCTCCTGT |
| | TGCTGTACCAAACCTTCGGACGGAAATTGCACCTGTATTCCCATCCCATCATCCTGGGCTTTC |
| | GGAAAATTCCTATGGGAGTGGGCCTCAGCCCGTTTCTCCTGGCTCAGTTTACTAGTGCCATTT |
| | GTTCAGTGGTTCGTAGGGCTTTCCCCCACTGTTTGGCTTTCAGTTATATGGATGATGTGGTAT |
| | TGGGGGCCAAGTCTGTACAGCATCTTGAGTCCCTTTTTACCGCTGTTACCAATTTTCTTTTGT |
| | CTTTGGGTATACATTTAAACCCTAACAAAACAAAGAGATGGGGTTACTCTCTAAATTTTATGG |
| | GTTATGTCATTGGATGTTATGGGTCCTTGCCACAAGAACACATCATACAAAAAATCAAAGAAT |
| | GTTTTAGAAAACTTCCTATTAACAGGCCTATTGATTGGAAAGTATGTCAACGAATTGTGGGTC |
| | TTTTGGGTTTTGCTGCCCCTTTTACACAATGTGGTTATCCTGCGTTGATGCCTTTGTATGCAT |
| | GTATTCAATCTAAGCAGGCTTTCACTTTCTCGCCAACTTACAAGGCCTTTCTGTGTAAACAAT |
| | ACCTGAACCTTTACCCCGTTGCCCGGCAACGGCCAGGTCTGTGCCAAGTGTTTGCTGACGCAA |
| | CCCCCACTGGCTGGGGCTTGGTCATGGGCCATCAGCGCATGCGTGGAACCTTTTCGGCTCCTC |
| | TGCCGATCCATACTGCGGAACTCCTAGCCGCTTGTTTTGCTCGCAGCAGGTCTGGAGCAAACA |
| | TTATCGGGACTGATAACTCTGTTGTCCTATCCCGCAAATATACATCGTTTCCATGGCTGCTAG |
| | GCTGTGCTGCCAACTGGATCCTGCGCGGGACGTCCTTTGTTTACGTCCCGTCGGCGCTGAATC |
| | CTGCGGACGACCCTTCTCGGGGTCGCTTGGGACTCTCTCGTCCCCTTCTCCGTCTGCCGTTCC |
| | GACCGACCACGGGGCGCACCTCTCTTTACGCGGACTCCCCGTCTGTGCCTTCTCATCTGCCGG |
| | ACCGTGTGCACTTCGCTTCACCTCTGCACGTCGCATGGAGACCACCGTGAACGCCCACCAAAT |
| | ATTGCCCAAGGTCTTACATAAGAGGACTCTTGGACTCTCAGCAATGTCAACGACCGACCTTGA |
| | GGCATACTTCAAAGACTGTTTGTTTAAAGACTGGGAGGAGTTGGGGGAGGAGATTAGGTTAAA |
| | GGTCTTTGTACTAGGAGGCTGTAGGCATAAATTGGTCTGCGCACCAGCACCATGCAACTTTTT |
| | CACCTCTGCCTAATCATCTCTTGTTCATGTCCTACTGTTCAAGCCTCCAAGCTGTGCCTTGGG |
| | TGGCTTTGGGGCATGGACATCGACCCTTATAAAGAATTTGGAGCTACTGTGGAGTTACTCTCG |
| | TTTTTGCCTTCTGACTTCTTTCCTTCAGTACGAGATCTTCTAGATACCGCCTCAGCTCTGTAT |
| | CGGGAAGCCTTAGAGTCTCCTGAGCATTGTTCACCTCACCATACTGCACTCAGGCAAGCAATT |
| | CTTTGCTGGGGGGAACTAATGACTCTAGCTACCTGGGTGGGTGTTAATTTGGAAGATCCAGCG |
| | TCTAGAGACCTAGTAGTCAGTTATGTCAACACTAATATGGGCCTAAAGTTCAGGCAACTCTTG |
| | TGGTTTCACATTTCTTGTCTCACTTTTGGAAGAGAAACAGTTATAGAGTATTTGGTGTCTTTC |
| | GGAGTGTGGATTCGCACTCCTCCAGCTTATAGACCACCAAATGCCCCTATCCTATCAACACTT |
| | CCGGAGACTACTGTTGTTAGACGACGAGGCAGGTCCCCTAGAAGAAGAACTCCCTCGCCTCGC |
| | AGACGAAGGTCTCAATCGCCGCGTCGCAGAAGATCTCAATCTCGGGAATCTCAATGTTAGTAT |
| | TCCTTGGACTCATAAGGTGGGGAACTTTACTGGGCTTTATTCTTCTACTGTACCTGTCTTTAA |
| | TCCTCATTGGAAAACACCATCTTTTCCTAATATACATTTACACCAAGACATTATCAAAAAATG |
| | TGAACAGTTTGTAGGCCCACTCACAGTTAATGAGAAAAGAAGATTGCAATTGATTATGCCTGC |
| | CAGGTTTTATCCAAAGGTTACCAAATATTTACCATTGGATAAGGGTATTAAACCTTATTATCC |
| | AGAACATCTAGTTAATCATTACTTCCAAACTAGACACTATTTACACACTCTATGGAAGGCGGG |
| | TATATTATATAAGAGAGAAACAACACATAGCGCCTCATTTTGTGGGTCACCATATTCTTGGGA |
| | ACAAGATCTACAGCATGGGGCAGAATCTTTCCACCAGCAATCCTCTGGGATTCTTTCCCGACC |
| | ACCAGTTGGATCCAGCCTTCAGAGCAAACACCGCAAATCCAGATTGGGACTTCAATCCCAACA |
| | AGGACACCTGGCCAGACGCCAACAAGGTAGGAGCTGGAGCATTCGGGCTGGGTTTCACCCCAC |
| | CGCACGGAGGCCTTTTGGGGTGGAGCCCTCAGGCTCAGGGCATACTACAAACTTTGCCAGCAA |
| | ATCCGCCTCCTGCCTCCACCAATCGCCAGTCAGGAAGGCAGCCTACCCCGCTGTCTCCACCTT |
| | TGAGAAACACTCATCCTCAGGCCATGCAGTGG |
| 2 | TTCCACTGCCTTCCACCAAGCTCTGCAGGATCCCAGAGTCAGGGGTCTGTATTTTCCTGCTGG |
| | TGGCTCCAGTTCAGGAACAGTAAACCCTGCTCCGAATATTGCCTCTCACATCTCGTCAATCTC |
| | CGCGAGGACTGGGGACCCTGTGACGAACATGGAGAACATCACATCAGGATTCCTAGGACCCCT |
| | GCTCGTGTTACAGGCGGGGTTTTTCTTGTTGACAAGAATCCTCACAATACCGCAGAGTCTAGA |
| | CTCGTGGTGGACTTCTCTCAATTTTCTAGGGGGATCACCCGTGTGTCTTGGCCAAAATTCGCA |
| | GTCCCCAACCTCCAATCACTCACCAACCTCCTGTCCTCCAATTTGTCCTGGTTATCGCTGGAT |
| | GTGTCTGCGGCGTTTTATCATATTCCTCTTCATCCTG--- |
| | CTGCTATGCCTCATCTTCTTATTGGTTCTTCTGGATTATCAAGGTATGTTGCCCGTTTGTCCT |
| | CTAATTCCAGGATCAACAACAACCAGTACGGGACCATGCAAAACCTGCACGACTCCTGCTCAA |
| | GGCAACTCTATGTTTCCCTCATGTTGCTGTACAAAACCTACGGATGGAAATTGCACCTGTATT |
| | CCCATCCCATCGTCCTGGGCTTTCGCAAAATACCTATGGGAGTGGGCCTCAGTCCGTTTCTCT |
| | TGGCTCAGTTTACTAGTGCCATTTGTTCAGTGGTTCGTAGGGCTTTCCCCCACTGTTTGGCTT |
| | TCAGCTATATGGATGATGTGGTATTGGGGGCCAAGTCTGTACAGCATCGTGAGTCCCTTTATA |
| | CCGCTGTTACCAATTTTCTTTTGTCTCTGGGTATACATTTAAACCCTAACAAAACAAAAAGAT |
| | GGGGTTATTCCCTAAACTTCATGGGTTACATAATTGGAAGTTGGGGAACATTGCCACAGGATC |
| | ATATTGTACAAAAGATCAAACACTGTTTTAGAAAACTTCCTGTTAACAGGCCTATTGATTGGA |
| | AAGTATGTCAAAGAATTGTGGGTCTTTTGGGCTTTGCTGCTCCATTTACACAATGTGGATATC |
| | CTGCCTTAATGCCTTTGTATGCATGTATACAAGCTAAACAGGCTTTCACTTTCTCGCCAACTT |
| | ACAAGGCCTTTCTAAGTAAACAGTACATGAACCTTTACCCCGTTGCTCGGCAACGGCCTGGTC |
| | TGTGCCAAGTGTTTGCTGACGCAACCCCCACTGGCTGGGGCTTGGCCATAGGCCATCAGCGCA |
| | TGCGTGGAACCTTTGTGGCTCCTCTGCCGATCCATACTGCGGAACTCCTAGCCGCTTGTTTTG |
| | CTCGCAGCCGGTCTGGAGCAAAGCTCATCGGAACTGACAATTCTGTCGTCCTCTCGCGGAAAT |
| | ATACATCGTTTCCATGGCTGCTAGGCTGTACTGCCAACTGGATCCTTCGCGGGACGTCCTTTG |
| | TTTACGTCCCGTCGGCGCTGAATCCCGCGGACGACCCCTCTCGGGGCCGCTTGGGACTCTCTC |
| | GTCCCCTTCTCCGTCTGCCGTTCCAGCCGACCACGGGGCGCACCTCTCTTTACGCGGTCTCCC |
| | CGTCTGTGCCTTCTCATCTGCCGGTCCGTGTGCACTTCGCTTCACCTCTGCACGTTGCATGGA |
| | GACCACCGTGAACG------ |
| | CCCATCAGATCCTGCCCAAGGTCTTACATAAGAGGACTCTTGGACTCCCAGCAATGTCAACGA |
| | CCGACCTTGAGGCCTACTTCAAAGACTGTGTGTTTAAGGACTGGGAGGAGCTGGGGGAGGAGA |
| | TTAGGTTAAAGGTCTTTGT------------ |
| | ATTAGGAGGCTGTAGGCATAAATTGGTCTGCGCACCAGCACCATGCAACTTTTTCACCTCTGC |
| | CTAATCATCTCTTGTACATGTCCCACTGTTCAAGCCTCCAAGCTGTGCCTTGGGTGGCTTTGG |
| | GGCATGGACATTGACCCTTATAAAGAATTTGGAGCTACTGTGGAGTTACTCTCGTTTTTGCCT |
| | TCTGACTTCTTTCCTTCCGTCAGAGATCTCCTAGACACCGCCTCAGCTCTGTATCGGGAAGCC |
| | TTAGAGTCTCCTGAGCATTGCTCACCTCACCATACTGCACTCAGGCAAGCCATTCTCTGCTGG |
| | GGGGAATTGATGACTCTAGCTACCTGGGTGGGTAATAATTTGGAAGATCCAGCATCCAGGGAT |
| | CTAGTAGTCAATTATGTTAATACTAACATGGGTTTAAAGATCAGGCAACTATTGTGGTTTCAT |
| | ATATCTTGCCTTACTTTTGGAAGAGAGACTGTACTTGAATATTTGGTCTCTTTCGGAGTGTGG |
| | ATTCGCACTCCTCCAGCCTATAGACCACCAAATGCCCCTATCTTATCAACACTTCCGGAAACT |
| | ACTGTTGTTAGACGACGGGACC--- |
| | GAGGCAGGTCCCCTAGAAGAAGAACTCCCTCGCCTCGCAGACGCAGATCTCAATCGCCGCGTC |
| | GCAGAAGATCTCAATCTCGGGAATCTCAATGTTAGTATTCCTTGGACTCATAAGGTGGGAAAC |
| | TTTACTGGGCTTTATTCCTCTACAGTACCTATCTTTAATCCTGAATGGCAAACTCCTTCCTTT |
| | CCTAAGATTCATTTACAAGAGGACATTATTAATAGGTGTCAACAATTTGTGGGCCCTCTCACT |
| | GTAAATGAAAAGAGAAGATTGAAATTAATTATGCCTGCTAGATTCTATCCTACCCACACTAAA |
| | TATTTGCCCTTAGACAAAGGAATTAAACCTTATTATCCAGATCAGGTAGTTAATCATTACTTC |
| | CAAACCAGACATTATTTACATACTCTTTGGAAGGCTGGTATTCTATATAAGAGGGAAACCACA |
| | CGTAGCGCATCATTTTGCGGGTCACCATATTCTTGGGAACAAGAGCTACAGCATGGGAGGTTG |
| | GTCATCAAAACCTCGCAAAGGCATGGGGACGAATCTTTCTGTTCCCAACCCTCTGGGATTCTT |
| | TCCCGATCATCAGTTGGACCCTGCATTCGGAGCCAACTCAAACAATCCAGATTGGGACTTCAA |
| | CCCCATCAAGGACCACTGGCCAGCAGCCAACCAGGTAGGAGTGGGAGCATTCGGGCCAGGGTT |
| | CACCCCTCCACACGGCGGTGTTTTGGGGTGGAGCCCTCAGGCTCAGGGCATATTGACCACAGT |
| | GTCAACAATTCCTCCTCCTGCCTCCACCAATCGGCAGTCAGGAAGGCAGCCTACTCCCATCTC |
| | TCCACCTCTAAGAGACAGTCATCCTCAGGCCATGCAGTGGAA------ |
| 3 | CTCCACCACTTTCCACCAAACTCTTCAAGATCCCAGAGTCAGGGCCCTGTACTTTCCTGCTGG |
| | TGGCTCCAGTTCAGGAACAGTGAGCCCTGCTCAGAATACTGTCTCTGCCATATCGTCAATCTT |
| | ATCGAAGACTGGGGACCCTGTACCGAACATGGAGAACATCGCATCAGGACTCCTAGGACCCCT |
| | GCTCGTGTTACAGGCGGGGTTTTTCTTGTTGACAAAAATCCTCACAATACCACAGAGTCTAGA |
| | CTCGTGGTGGACTTCTCTCAATTTTCTAGGGGGAACACCCGTGTGTCTTGGCCAAAATTCGCA |
| | GTCCCAAATCTCCAGTCACTCACCAACCTGTTGTCCTCCAATTTGTCCTGGTTATCGCTGGAT |
| | GTGTCTGCGGCGTTTTATCATCTTCCTCTGCATCCTGCTGCTATGCCTCATCTTCTTGTTGGT |
| | TCTTCTGGACTATCAAGGTATGTTGCCCGTTTGTCCTCTAATTCCAGGATCATCAACAACCAG |
| | CACCGGACCATGCAAAACCTGCACAACTCCTGCTCAAGGAACCTCTATGTTTCCCTCATGTTG |
| | CTGTACAAAACCTACGGACGGAAACTGCACCTGTATTCCCATCCCATCATCTTGGGCTTTCGC |
| | AAAATACCTATGGGAGTGGGCCTCAGTCCGTTTCTCTTGGCTCAGTTTACTAGTGCCATTTGT |
| | TCAGTGGTTCGTAGGGCTTTCCCCCACTGTCTGGCTTTCAGTTATATGGATGATGTGGTTTTG |
| | GGGGCCAAGTCTGTACAACATCTTGAGTCCCTTTATGCCGCTGTTACCAATTTTCTTTTGTCT |
| | TTGGGTATACATTTAAACCCTCACAAAACAAAAAGATGGGGATATTCCCTTAACTTCATGGGA |
| | TATGTAATTGGGAGTTGGGGCACATTGCCACAGGAACATATTGTACAAAAAATCAAAATGTGT |
| | TTTAGGAAACTTCCTGTAAACAGGCCTATTGATTGGAAAGTATGTCAACGAATTGTGGGTCTT |
| | TTGGGGTTTGCCGCCCCTTTCACGCAATGTGGATATCCTGCTTTAATGCCTTTATATGCATGT |
| | ATACAAGCAAAACAGGCTTTTACTTTCTCGCCAACTTACAAGGCCTTTCTAAGTAAACAGTAT |
| | CTGAACCTTTACCCCGTTGCTCGGCAACGGCCTGGTCTGTGCCAAGTGTTTGCTGACGCAACC |
| | CCCACTGGTTGGGGCTTGGCCATAGGCCATCAGCGCATGCGTGGAACCTTTGTGTCTCCTCTG |
| | CCGATCCATACTGCGGAACTCCTAGCCGCTTGTTTTGCTCGCAGCAGGTCTGGGGCAAAACTC |
| | ATCGGGACTGACAATTCTGTCGTGCTCTCCCGCAAGTATACATCATTTCCATGGCTGCTAGGC |
| | TGTGCTGCCAACTGGATCCTGCGCGGGACGTCCTTTGTTTACGTCCCGTCGGCGCTGAATCCC |
| | GCGGACGACCCCTCCCGGGGCCGCTTGGGGCTCTACCGCCCGCTTCTCCGCCTGTTGTACCGA |
| | CCGACCACGGGGCGCACCTCTCTTTACGCGGACTCCCCGTCTGTGCCTTCTCATCTGCCGGAC |
| | CGTGTGCACTTCGCTTCACCTCTGCACGTCGCATGGAGACCACCGTGAACGCCCACCGGAAC- |
| | --------CTGC--------------------- |
| | CCAAGGTCTTGCATAAGAGGACTCTTGGACTTTCAGCAATGTCAACGACCGACCTTGAGGCAT |
| | ACTTCAAAGACTGTGTGTTTACTGAGTGGGAGGAGTTGGGGGAGGAGATTAGGTTAAAGGTCT |
| | ---TTGTACTAGGAGGCTGTAGGCATAAATTGGTGTGTTCACCAGCACCATGCAACTTT--- |
| | TT--------------------------------------------------------- |
| | CACCTCTGCCTAATCATCTCATGTTCATGTCCTACTGTTCAAGCCTCCAAGCTGTGCCTTGGG |
| | TGGCTTTGGGGCATG------------------------------------ |
| | GACATTGACCCGTATAAAGAATTTGGAGCTTCTGTGGAGTTACTCTCTT------ |
| | TTTTGCCTTCTGACTTCTTTCCTTCTATTCGAGATCTCCTCGACACCGCCTCTGCTCTGTATC |
| | GGGAGGCCTTAGAGTCTCCGGAACATTGTTCACCTCACCATACGGCACTCAGGCAAGCTATTC |
| | TGTGTTGGGGTGAGTTGATGAATCTAGCCACCTGGGTGGGAAGTAATTTGGAAGATCCAGCAT |
| | CCAGGGAATTAGTAGTCAGCTATGTCAACGTTAATATGGGCCTAAAAATCAGACAACTATTGT |
| | GGTTTCACATTTCCTGTCTTACTTTTGGGAGAGAAACTGTTCTTGAATATTTGGTGTCTTTTG |
| | GAGTGTGGATTCGCACTCCTCCTGCATATAGACCACCAAATGCCCCTATCTTATCAACACTTC |
| | CGGAAACTACTGTTGTTAGACGAAG------ |
| | AGGCAGGTCCCCTAGAAGAAGAACTCCCTCGCCTCGCAGACGAAGGTCTCAATCGCCGCGTCG |
| | CAGAAGATCTCAATCTCGGGAA--- |
| | TCTCAATGTTAGTATTCCTTGGACACATAAGGTGGGAAACTTTACGGGGCTTTATTCTTCTAC |
| | GGTACCTTGCTTTAATCCTAAATGGCAAACTCCTTCTTTTCCTGACATTCATTTGCAGGAGGA |
| | CATTGTTGATAGATGTAAGCAATTTGTGGGGCCCCTTACAGTAAATGAAAACAGGAGACTAAA |
| | ATTAATTATGCCTGCTAGGTTTTATCCCAATGTTACTAAATATTTGCCCTTAGATAAAGGGAT |
| | CAAACCGTATTATCCAGAGCATGTAGTTAATCATTACTTCCAGACGAGACATTATTTACACAC |
| | TCTTTGGAAGGCGGGTATCTTATATAAAAGAGAGTCCACACGTAGCGCCTCATTTTGCGGGTC |
| | ACCATATTCTTGGGAACAAGATCTACAGCATGGGAGGTTGGTCTTCCAAACCTCGAAAAGGCA |
| | TGGGGACAAATCTTTCTGTCCCCAATCCCCTGGGATTCTTCCCCGATCATCAGTTGGACCCTG |
| | CATTCAAAGCCAACTCAGAAAATCCAGATTGGGACCTCAACCCGCACAAGGACAACTGGCCGG |
| | ACGCCAACAAGGTGGGAGTGGGAGCATTCGGGCCAGGGTTCACCCCTCCCCATGGGGGACTGT |
| | TGGGGTGGAGCCCTCAGGCTCAGGGCATACTCACAACTGTGCCAGCAGCTCCTCCTCCTGCCT |
| | CCACCAATCGGCAGTCAGGAAGGCAGCCTACTCCCTTATCTCCACCTCTAAGGGACACTCATC |
| | CTCAGGCCATGCAGTGGAA |
| 4 | CTCCACAACATTCCACCAAGCTCTGCTAGATCCCAGAGTGAGGGGCCTATACTTTCCTGCTGG |
| | TGGCTCCAGTTCCGGAACAGTAAACCCTGTTCCGACTACTGCCTCACCCATATCGTCAATCTT |
| | CTCGAGGACTGGGGACCCTGCACCGAACATGGAGAACACAACATCAGGATTCCTAGGACCCCT |
| | GCTCGTGTTACAGGCGGGGTTTTTCTTGTTGACAAGAATCCTCACAATACCACAGAGTCTAGA |
| | CTCGTGGTGGACTTCTCTCAATTTTCTAGGGGGAGCACCCACGTGTCCTGGCCAAAATTCGCA |
| | GTCCCCAACCTCCAATCACTCACCAACCTCTTGTCCTCCAATTTGTCCTGGCTATCGCTGGAT |
| | GTGTCTGCGGCGTTTTATCATATTCCTCTTCATCCTGCTGCTATGCCTCATCTTCTTGTTGGT |
| | TCTTCTGGACTACCAAGGTATGTTGCCCGTTTGTCCTCTACTTCCAGGAACATCAA---CT-- |
| | ------- |
| | ACCAGCACGGGACCATGCAAGACCTGCACGATTCCTGCTCAAGGAACCTCTATGTTTCCCTCT |
| | TGTTGCTGTACAAAACCTTCGGACGGAAACTGCACTTGTATTCCCATCCCATCATCCTGGGCT |
| | TTCGCAAGATTCCTATGGGAGTGGGCCTCAGTCCGTTTCTCCTGGCTCAGTTTACTAGTGCCA |
| | TTTGTTCAGTGGTTCGTAGGGCTTTCCCCCACTGTTTGGCTTTCAGTTATATGGATGATGTGG |
| | TATTGGGGGCCAAGTCTGTACAACATCTTGAGTCCCTTTTTACCTCTATTACCAATTTTCTTT |
| | TGTCTTTGGGTATACATTTGAACCCTAATAAAACCAAACGTTGGGGCTACTCCCTTAACTTCA |
| | TGGGATATGTAATTGGAAGTTGGGGTACTTTACCACAGGAACATATTGTACTAAAAATCAAGC |
| | AATGTTTTCGAAAACTGCCTGTAAATAGACCTATTGATTGGAAAGTATGTCAAAGAATTGTGG |
| | GTCTTTTGGGCTTTGCTGCCCCTTTTACACAATGTGGCTATCCTGCCTTAATGCCTTTATATG |
| | CATGTATACAATCTAAGCAGGCTTTCACTTTCTCGCCAACTTACAAGGCCTTTCTGTGTAAAC |
| | AATATCTGAACCTTTACCCCGTTGCCCGGCAACGGTCAGGTCTCTGCCAAGTGTTTGCTGACG |
| | CAACCCCCACTGGATGGGGCTTGGCCATAGGCCATCGGCGCATGCGTGGAACCTTTGTGGCTC |
| | CTCTGCCGATCCATACTGCGGAACTCCTAGCAGCTTGTTTTGCTCGCAGCCGGTCTGGAGCGA |
| | AACTTATCGGAACCGACAACTCTGTTGTCCTCTCTCGGAAATACACCTCCTTTCCATGGCTGC |
| | TAGGGTGTGCTGCCAACTGGATCCTGCGCGGGACGTCCTTTGTCTACGTCCCGTCGGCGCTGA |
| | ATCCCGCGGACGACCCGTCTCGGGGCCGTTTGGGACTCTACCGTCCCCTTCTTCATCTGCCGT |
| | TCCGGCCGACCACGGGGCGCACCTCTCTTTACGCGGTCTCCCCGTCTGTGCCTTCTCATCTGC |
| | CGGACCGTGTGCACTTCGCTTCACCTCTGCACGTCGCATGGAGACCACCGTGAACGCCCACCA |
| | GGTCT---CC-A--AGGT-TTGC--------- |
| | CCAAGGTCTTACATAAGAGGACTCTTGGACTCTCAGCAATGTCAACGACCGACCTTGAGGCAT |
| | ACTTCAAAGACTGTTTGTTTAAAGACTGGGAGGAGTTGGGGGAGGAGATTAGGTTAATGATCT |
| | ---TTGTACTAGGAGGCTGTAGGCATAAATTGGTCTGTTCACCAGCACCATGCAA-------- |
| | ------------------------------------------------- |
| | CTTTTTCACCTCTGCCTAATCATCTCATGTTCATGTCCTACTGTTCAAGCCTCCAAGCTGTGC |
| | CTTGGGTGGCTTTGGGGCATGGACATTGACCCGTATAAAGAATTTGGAGCTTCTGTGGAGTTA |
| | CTCTCTT--- |
| | TTTTGCCTTCTGACTTCTTTCCTTCTATTCGAGATCTCCTCGACACCGCCTCTGCTCTGTATC |
| | GGGAGGCCTTAGAGTCTCCGGAACATTGTTCACCTCACCATACAGCACTCAGGCAAGCTATTC |
| | TGTGTTGGGGTGAGTTGATGAATCTGGCCACCTGGGTGGGAAGTAATTTGGAAGACCCAGCAT |
| | CCAGGGAATTAGTAGTCAGCTATGTCAATGTTAATATGGGCCTAAAAATCAGACAACTATTGT |
| | GGTTTCACATTTCCTGTCTTACTTTTGGAAGAGAAACTGTTCTTGAGTATTTGGTGTCTTTTG |
| | GAGTGTGGATTCGCACTCCTCCCGCTTACAGACCACCAAATGCCCCTATCTTATCAACACTTC |
| | CGGAAACTACTGTTGTTAGA------ |
| | CGACGAGGCAGGTCCCCTAGAAGAAGAACTCCCTCGCCTCGCAGACGAAGGTCTCAATCGCCG |
| | CGTCGCAGAAGATCTCAATCTCGGGAATCTCAATGTTAGTATCCCTTGGACTCATAAGGTGGG |
| | AAACTTTACTGGGCTTTATTCTTCTACTGTACCTGTCTTTAATCCTGAGTGGCAAACTCCCTC |
| | CTTTCCTCACATTCATTTACAGGAGGACATTATTAATAGATGTCAACAATATGTGGGCCCTCT |
| | TACAGTTAATGAAAAAAGGAGATTAAAATTAATTATGCCTGCTAGGTTCTATCCTAACCTTAC |
| | CAAATATTTGCCCTTGGACAAAGGCATTAAACCTTATTATCCTGAACATGCAGTTAATCATTA |
| | CTTCAAAACTAGGCATTATTTACATACTCTGTGGAAGGCTGGCATTCTATATAAGAGAGAAAC |
| | TACACGCAGCGCCTCATTTTGTGGGTCACCATATTCTTGGGAACAAGAGCTACAGCATGGGAG |
| | GTTGGTCTTCCAAACCTCGACAAGGCATGGGGACGAATCTTTCTGTTCCCAATCCTCTGGGAT |
| | TCTTTCCCGATCACCAGTTGGACCCTGCGTTCGGAGCCAACTCAAACAATCCAGATTGGGACT |
| | TCAACCCCAACAAGGATCACTGGCCAGAGGCAAATCAGGTAGGAGCGGGAGCATTCGGGCCAG |
| | GGTTCACCCCACCACACGGCG------------ |
| | GTCTTTTGGGGTGGAGCCCTCAGGCTCAGGGCATATTGACAACA------------------- |
| | ----- |
| | GTGCCAGCAGCACCTCCTCCTGCCTCCACCAATCGGCAGTCAGGAAGACAGCCTACTCCCATC |
| | TCTCCACCTCTAAGAGACAGTCATCCTCAGGCCATGCAGTGGAA |
| 5 | --- |
| | CTCCACCACATTCCACCAAGCTCTGCTAGATCCCAGAGTGAGGGGCCTATACTTTCCTGCTGG |
| | TGGCTCCAGTTCCGGAACAGTAAACCCTGTTCCGACTACTGCCTCTCCCATATCGTCAATCTT |
| | CTCGAGGACTGGGGACCCTGCACCGAACATGGAGAACACCACATCAGGATTCCTAGGACCCCT |
| | GCTCGTGTTACAGGCGGGGTTTTTCTTGTTGACAAGAATCCTCACAATACCACAGAGTCTAGA |
| | CTCGTGGTGGACTTCTCTCAATTTTCTAGGGGGAGCACCCACGTGTCCTGGCCAAAATTCGCA |
| | GTCCCCAACCTCCAATCACTCACCAACCTCTTGTCCTCCAATTTGTCCTGGTTATCGCTGGAT |
| | GTGTCTGCGGCGTTTTATCATCTTCCTCTTCATCCTGCTGCTATGCCTCATCTTCTTGTTGGT |
| | TCTTCTGGACTACCAAGGTATGTTGCCCGTTTGTCCTCTACTTCCAGGAACATCAAC-ACC-- |
| | --------- |
| | CAGCACGGGACCATGCAAGACCTGCACGACTCCTGCTCAAGGAACCTCTATGTTTCCCTCTTG |
| | TTGCTGTACAAAACCTTCGGACGGAAACTGCACTTGTATTCCCATCCCATCATCTTGGGCTTT |
| | CGCAAGATTCCTATGGGAGTGGGCCTCAGTCCGTTTCTCCTGGCTCAGTTTACTAGTGCCATT |
| | TGTTCAGTGGTTCGTAGGGCTTTCCCCCACTGTTTGGCTTTCAGTTATATGGATGATGTGGTA |
| | TTGGGGGCCAAGTCTGTACAACATCTTGAGTCCCTTTTTACCGCTATTACCAATTTTCTTTTG |
| | TCTTTGGGTATACATTTAAACCCTAATAAAACCAAACGTTGGGGCTACTCCCTTAACTTCATG |
| | GGATATGTAATTGGAAGTTGGGGTACTTTACCACAGGAACATATTGTACAAAAAATCAAACAA |
| | TGTTTTCGGAAACTTCCTGTAAATAGACCTATTGATTGGAAAGTATGTCAAAGAATTGTGGGT |
| | CTTTTGGGCTTTGCTGCCCCTTTTACACAATGTGGCTATCCTGCTTTAATGCCTTTATATGCA |
| | TGTATACAAGCTAAGCAGGCTTTCACTTTCTCGCCAACTTACAAGGCCTTTCTGTGTAAACAA |
| | TATCTGAACCTTTACCCCGTTGCTCGGCAACGGTCAGGTCTGTGCCAAGTGTTTGCTGACGCA |
| | ACCCCCACTGGTTGGGGCTTGGCCATAGGCCATCAGCGCATGCGTGGAACCTTTGTGGCTCCT |
| | CTGCCGATCCATACTGCGGAACTCCTAGCAGCTTGTTTTGCTCGCAGCCGGTCTGGAGCAAAA |
| | CTTATCGGGACTGACAACTCTGTTGTCCTCTCTCGGAAATACACCTCCTTTCCATGGCTGCTA |
| | GGCTGTGCTGCCAACTGGATCCTGCGCGGGACGTCCTTTGTCTACGTCCCGTCGGCGCTGAAT |
| | CCCGCGGACGACCCGTCTCGGGGCCGTTTGGGGCTCTACCGTCCCCTTCTTCGTCTGCCGTTC |
| | CGGCCGACCACGGGGCGCACCTCTCTTTACGCGGTCTCCCCGTCTGTGCCTTCTCATCTGCCG |
| | GACCGTGTGCACTTCGCTTCACCTCTGCACGTCGCATGGAGACCACCGTGAACGCCCACCGGA |
| | ACA---CC---CTGGT--------------------- |
| | CAAGGTCTTACATAAGAGGACTCTTGGACTCTCAGCAATGTCAACGACCGACCTTGAGGCATA |
| | CTTCAAAGACTGTGTGTTTAATGACTGGGAGGAGTTGGGGGAGGAGATTAGGTTAAAG-- |
| | CTTCTTTGTACTAGGAGGCTGTAGGCATAAATTGGTCTGTTCACCAGCACCATGCAACTTTT- |
| | ----------------------------------------------------------- |
| | TCACCTCTGCCTAATCATCTCATGTTCATGTCCTACTGTTCAAGCCTCCAAGCTGTGCCTTGG |
| | GTGGCTTTGGGG------------------------------------ |
| | CATGGACATTGACCCGTATAAAGAATTTGGAGCTTCTGTGGAGTTACTCTCTT------ |
| | TTTTGCCTTCTGACTTCTTTCCTTCTATTCGAGATCTCCTCGACACCGCCTCTGCTCTGTATC |
| | GGGAGGCCTTAGAGTCTCCGGAACATTGTTCACCTCACCATACAGCACTCAGGCAAGCTATTC |
| | TGTGTTGGGGTGAGTTGATGAATCTGGCCACCTGGGTGGGAAGTAATTTGGAAGACCCAGCAT |
| | CCAGGGAATTAGTAGTCAGCTATGTCAATGTTAATATGGGCCTAAAAATCAGACAACTATTGT |
| | GGTTTCACATTTCCTGTCTTACTTTTGGAAGAGAAACTGTTCTTGAGTATTTGGTGTCTTTTG |
| | GAGTGTGGATTCGCACTCCTCCTGCTTACAGACCACCAAATGCCCCTATCTTATCAACACTTC |
| | CGGAAACTACTGTTGTTAGACG------ |
| | ACGAGGCAGGTCCCCTAGAAGAAGAACTCCCTCGCCTCGCAGACGAAGGTCTCAATCGCCGCG |
| | TCGCAGAAGATCTCAATCTCGGGA--- |
| | ATCTCAATGTTAGTATCCCTTGGACTCATAAGGTGGGAAACTTTACTGGGCTTTATTCTTCTA |
| | CTGTACCTGTCTTTAATCCTGAGTGGCAAACTCCCTCTTTTCCTCACATTCATTTGCAGGAGG |
| | ACATTATTAATAGATGTCAACAATATGTGGGCCCTCTTACAGTTAATGAAAAAAGGAGATTAA |
| | AATTAATTATGCCTGCTAGGTTCTATCCTAACCTTACCAAATATTTGCCCTTAGATAAAGGCA |
| | TTAAACCTTATTATCCTGAACATGCAGTTAATCATTACTTCCAAACTAGGCATTATTTACATA |
| | CTCTGTGGAAGGCGGGCATTTTATATAAGAGAGAAACTACACGCAGCGCCTCATTTTGTGGGT |
| | CACCATATTCTTGGGAACAAGAGCTACAGCATGGGAGGTTGGTCTTCCAAACCTCGACAAGGC |
| | ATGGGGACAAATCTTTCTGTTCCCAATCCTCTGGGATTCTTTCCCGATCACCAGTTGGACCCT |
| | GCATTCGGAGCCAACTCAAACAATCCAGATTGGGACTTCAACCCCAACAAGGATCACTGGCCA |
| | GAGGCAAACCAGGTAGGAGTGGGAGCATTCGGGCCAGGGTTCACCCCACCACACGGCGG---- |
| | --------TCTTTTGGGGTGGAGCCCTCAGGCTCAGGGCATATTGACAACA------------ |
| | ------------ |
| | GTGCCAGCAGCTCCTCCTCCTGCCTCCACCAATCGGCAGTCAGGAAGACAGCCTACTCCCATC |
| | TCTCCACCTCTAAGAGACAGTCATCCTCAGGCCATGCAGTGGAA |
| 6 | CACCAAACTCTTCAAGATCC |
| 7 | CAGGAACAGTGAGCCCTGCT |
| 8 | ACCCCTGCTCGTGTTACAGG |
| 9 | CAAAAATCCTCACAATACCA |
| 10 | CCAATTTGTCCTGGTTATCG |
| 11 | GCCCGTTTGTCCTCTAATTC |
| 12 | GGCTTTCGCAAAATACCTAT |
| 13 | ACTGTCTGGCTTTCAGTTAT |
| 14 | GGCCAAGTCTGTACAACATC |
| 15 | TTACCAATTTTCTTTTGTCT |
| 16 | CCCTCACAAAACAAAAAGAT |
| 17 | GGGATATTCCCTTAACTTCA |
| 18 | ACTTCATGGGATATGTAATT |
| 19 | ATTGATTGGAAAGTATGTCA |
| 20 | GGAAAGTATGTCAACGAATT |
| 21 | TCAACGAATTGTGGGTCTTT |
| 22 | TGCCGCCCCTTTCACGCAAT |
| 23 | CTGCTAGGCTGTGCTGCCAA |
| 24 | TTGTTTACGTCCCGTCGGCG |
| 25 | CTTCAAAGACTGTGTGTTTA |
| 26 | CTGTGTGTTTACTGAGTGGG |
| 27 | TCAAGCCTCCAAGCTGTGCC |
| 28 | TAAAGAATTTGGAGCTTCTG |
| 29 | TGCTCTGTATCGGGAGGCCT |
| 30 | TCAGGCAAGCTATTCTGTGT |
| 31 | TTCTGTGTTGGGGTGAGTTG |
| 32 | GAGTTGATGAATCTAGCCAC |
| 33 | TTTGGAAGATCCAGCATCCA |
| 34 | TTTTGGGAGAGAAACTGTTC |
| 35 | CTTGAATATTTGGTGTCTTT |
| 36 | TATTTGGTGTCTTTTGGAGT |
| 37 | AAATATTTGCCCTTAGATAA |
| 38 | ATTTACACACTCTTTGGAAG |
| 39 | GGCGGGTATCTTATATAAAA |
| 40 | CACACGTAGCGCCTCATTTT |
| 41 | TCTTTCTGTCCCCAATCCCC |
| 42 | TGGCCGGACGCCAACAAGGT |
| 43 | GGGAGTGGGAGCATTCGGGC |
| 44 | CCCTCCCCATGGGGGACTGT |
| 45 | GCCCTGACTCTGGGATCTTG |
| 46 | AAAGTACAGGGCCCTGACTC |
| 47 | GATGTTCTCCATGTTCGGTA |
| 48 | GTAACACGAGCAGGGGTCCT |
| 49 | ACCCCGCCTGTAACACGAGC |
| 50 | TCTAGACTCTGTGGTATTGT |
| 51 | AAAATTGAGAGAAGTCCACC |
| 52 | GCGAATTTTGGCCAAGACAC |
| 53 | GTGACTGGAGATTTGGGACT |
| 54 | AATTGGAGGACAACAGGTTG |
| 55 | AAGAAGATGAGGCATAGCAG |
| 56 | GTGCTGGTTGTTGATGATCC |
| 57 | AACATAGAGGTTCCTTGAGC |
| 58 | AAAGCCCAAGATGATGGGAT |
| 59 | TTTGCGAAAGCCCAAGATGA |
| 60 | TCCCCATCTTTTTGTTTTGT |
| 61 | TACATATCCCATGAAGTTAA |
| 62 | TGCATATAAAGGCATTAAAG |
| 63 | ACCAGGCCGTTGCCGAGCAA |
| 64 | ATGGCCAAGCCCCAACCAGT |
| 65 | GCGGCTAGGAGTTCCGCAGT |
| 66 | TGCGAGCAAAACAAGCGGCT |
| 67 | CGACAGAATTGTCAGTCCCG |
| 68 | ATACTTGCGGGAGAGCACGA |
| 69 | TAAACAAAGGACGTCCCGCG |
| 70 | GCCCCGGGAGGGGTCGTCCG |
| 71 | GAGCCCCAAGCGGCCCCGGG |
| 72 | CAGATGAGAAGGCACAGACG |
| 73 | GTTGACATTGCTGAAAGTCC |
| 74 | AGAAGCTCCAAATTCTTTAT |
| 75 | GAGGCGGTGTCGAGGAGATC |
| 76 | CCCAACACAGAATAGCTTGC |
| 77 | TTCATCAACTCACCCCAACA |
| 78 | GACTACTAATTCCCTGGATG |
| 79 | CATAGCTGACTACTAATTCC |
| 80 | GGTCTATATGCAGGAGGAGT |
| 81 | TTTGGTGGTCTATATGCAGG |
| 82 | GACCTTCGTCTGCGAGGCGA |
| 83 | TTTCCCACCTTATGTGTCCA |
| 84 | ATTAAAGCAAGGTACCGTAG |
| 85 | AAAAGAAGGAGTTTGCCATT |
| 86 | AAATGAATGTCAGGAAAAGA |
| 87 | TCAACAATGTCCTCCTGCAA |
| 88 | GGGCAAATATTTAGTAACAT |
| 89 | GTAATGATTAACTACATGCT |
| 90 | TATAAGATACCCGCCTTCCA |
| 91 | ATGCTGTAGATCTTGTTCCC |
| 92 | ATGATCGGGGAAGAATCCCA |
| 93 | GGTCCAACTGATGATCGGGG |
| 94 | TTCTGAGTTGGCTTTGAATG |
| 95 | CTGGATTTTCTGAGTTGGCT |
| 96 | GAGGTCCCAATCTGGATTTT |
| 97 | GTGCGGGTTGAGGTCCCAAT |
| 98 | GTCCGGCCAGTTGTCCTTGT |
| 99 | GGGGAGGGGTGAACCCTGGC |
| 100 | CCCAACAGTCCCCCATGGGG |
| 101 | GAGGAGCTGCTGGCACAGTT |
| 102 | TCCCTTAGAGGTGGAGATAA |
| 103 | CACCAAGCTCTGCTAGATCC |
| 104 | GAACATGGAGAACACAACAT |
| 105 | GCGGGGTTTTTCTTGTTGAC |
| 106 | CAAGAATCCTCACAATACCA |
| 107 | CCAATTTGTCCTGGCTATCG |
| 108 | AACCTCGACAAGGCATGGGG |
| 109 | GGCTTTCGCAAGATTCCTAT |
| 110 | ACTGTTTGGCTTTCAGTTAT |
| 111 | GGGCTACTCCCTTAACTTCA |
| 112 | TGGGATATGTAATTGGAAGT |
| 113 | GGAAAGTATGTCAAAGAATT |
| 114 | GTTTGCTGACGCAACCCCCA |
| 115 | CACCTCCTTTCCATGGCTGC |
| 116 | CTGCTAGGGTGTGCTGCCAA |
| 117 | TTGTCTACGTCCCGTCGGCG |
| 118 | CTGTTTGTTTAAAGACTGGG |
| 119 | CATGGACATTGACCCGTATA |
| 120 | GAGTTGATGAATCTGGCCAC |
| 121 | TTTGGAAGACCCAGCATCCA |
| 122 | TTTTGGAAGAGAAACTGTTC |
| 123 | CTTGAGTATTTGGTGTCTTT |
| 124 | TCGCAGAAGATCTCAATCTC |
| 125 | TACTGTACCTGTCTTTAATC |
| 126 | TACACGCAGCGCCTCATTTT |
| 127 | TCTTTCTGTTCCCAATCCTC |
| 128 | ATTGGGACTTCAACCCCAAC |
| 129 | AGGAGCGGGAGCATTCGGGC |
| 130 | TGGGATCTAGCAGAGCTTGG |
| 131 | AAAGTATAGGCCCCTCACTC |
| 132 | GGGTGAGGCAGTAGTCGGAA |
| 133 | TCCTCGAGAAGATTGACGAT |
| 134 | TGTGTTCTCCATGTTCGGTG |
| 135 | GCGAATTTTGGCCAGGACAC |
| 136 | GTGATTGGAGGTTGGGGACT |
| 137 | AATTGGAGGACAAGAGGTTG |
| 138 | GGCATAGCAGCAGGATGAAG |
| 139 | AAAGCCCAGGATGATGGGAT |
| 140 | CTTGCGAAAGCCCAGGATGA |
| 141 | ATAGGAATCTTGCGAAAGCC |
| 142 | GGACTGAGGCCCACTCCCAT |
| 143 | GCCCCAACGTTTGGTTTTAT |
| 144 | TGCATATAAAGGCATTAAGG |
| 145 | ACCTGACCGTTGCCGGGCAA |
| 146 | ATGGCCAAGCCCCATCCAGT |
| 147 | GCTGCTAGGAGTTCCGCAGT |
| 148 | TGCGAGCAAAACAAGCTGCT |
| 149 | GTATTTCCGAGAGAGGACAA |
| 150 | TAGACAAAGGACGTCCCGCG |
| 151 | GCCCCGAGACGGGTCGTCCG |
| 152 | GAGTCCCAAACGGCCCCGAG |
| 153 | GCAGATGAAGAAGGGGACGG |
| 154 | GTTGACATTGCTGAGAGTCC |
| 155 | GGTCGGTCGTTGACATTGCT |
| 156 | GGTCTGTAAGCGGGAGGAGT |
| 157 | TTTGGTGGTCTGTAAGCGGG |
| 158 | GTTTGAGTTGGCTCCGAACG |
| 159 | TTTCCCACCTTATGAGTCCA |
| 160 | CCAGTAAAGTTTCCCACCTT |
| 161 | ATTAAAGACAGGTACAGTAG |
| 162 | AAAGGAGGGAGTTTGCCACT |
| 163 | AAATGAATGTGAGGAAAGGA |
| 164 | TTAATAATGTCCTCCTGTAA |
| 165 | GGGCAAATATTTGGTAAGGT |
| 166 | GTAATGATTAACTGCATGTT |
| 167 | TATAGAATGCCAGCCTTCCA |
| 168 | CGTGTAGTTTCTCTCTTATA |
| 169 | ATGCTGTAGCTCTTGTTCCC |
| 170 | GTGATCGGGAAAGAATCCCA |
| 171 | GGTCCAACTGGTGATCGGGA |
| 172 | GAAGTCCCAATCTGGATTGT |
| 173 | GTTGGGGTTGAAGTCCCAAT |
| 174 | CTCTGGCCAGTGATCCTTGT |
| 175 | GTGGTGGGGTGAACCCTGGC |
| 176 | TCTCTTAGAGGTGGAGAGAT |
| 177 | CTGCCTTCCACCAAGCTCTG |
| 178 | CACCAAGCTCTGCAGGATCC |
| 179 | CTCTGCAGGATCCCAGAGTC |
| 180 | CAGGAACAGTAAACCCTGCT |
| 181 | GAACATGGAGAACATCACAT |
| 182 | GAAGTTGGGGAACATTGCCA |
| 183 | CAAGAATCCTCACAATACCG |
| 184 | GACTTCTCTCAATTTTCTAG |
| 185 | TCATCTTCTTATTGGTTCTT |
| 186 | AACCTCGCAAAGGCATGGGG |
| 187 | CATGTTGCTGTACAAAACCT |
| 188 | ACTGTTTGGCTTTCAGCTAT |
| 189 | GGCCAAGTCTGTACAGCATC |
| 190 | ACCCTAACAAAACAAAAAGA |
| 191 | GGGGTTATTCCCTAAACTTC |
| 192 | TGCTGCTCCATTTACACAAT |
| 193 | CTGCTAGGCTGTACTGCCAA |
| 194 | CATGGACATTGACCCTTATA |
| 195 | TAAAGAATTTGGAGCTACTG |
| 196 | AGCTCTGTATCGGGAAGCCT |
| 197 | GCAAGCCATTCTCTGCTGGG |
| 198 | GAATTGATGACTCTAGCTAC |
| 199 | ATTTGGAAGATCCAGCATCC |
| 200 | TCAATTATGTTAATACTAAC |
| 201 | TTTTGGAAGAGAGACTGTAC |
| 202 | CTTGAATATTTGGTCTCTTT |
| 203 | TATTTGGTCTCTTTCGGAGT |
| 204 | TACAGTACCTATCTTTAATC |
| 205 | AAATATTTGCCCTTAGACAA |
| 206 | CACACGTAGCGCATCATTTT |
| 207 | TCTTTCTGTTCCCAACCCTC |
| 208 | TGGCCAGCAGCCAACCAGGT |
| 209 | AGGAGTGGGAGCATTCGGGC |
| 210 | CCCTCCACACGGCGGTGTTT |
| 211 | AAAATACAGACCCCTGACTC |
| 212 | GTGAGAGGCAATATTCGGAG |
| 213 | GATGTTCTCCATGTTCGTCA |
| 214 | TCTAGACTCTGCGGTATTGT |
| 215 | AATTGGAGGACAGGAGGTTG |
| 216 | GTACTGGTTGTTGTTGATCC |
| 217 | AACATAGAGTTGCCTTGAGC |
| 218 | ACAGCAACATGAGGGAAACA |
| 219 | GTTTGAGTTGGCTCCGAATG |
| 220 | AAAGCCCAGGACGATGGGAT |
| 221 | TTTGCGAAAGCCCAGGACGA |
| 222 | ACCCCATCTTTTTGTTTTGT |
| 223 | TATGTAACCCATGAAGTTTA |
| 224 | TGCATACAAAGGCATTAAGG |
| 225 | ATGGCCAAGCCCCAGCCAGT |
| 226 | ATATTTCCGCGAGAGGACGA |
| 227 | GCCCCGAGAGGGGTCGTCCG |
| 228 | GAGTCCCAAGCGGCCCCGAG |
| 229 | GCAGACGGAGAAGGGGACGA |
| 230 | TCTAAGGGCAAATATTTAGT |
| 231 | CTCTTATGTAAGACCTTGGG |
| 232 | GTTGACATTGCTGGGAGTCC |
| 233 | AGTAGCTCCAAATTCTTTAT |
| 234 | AGAAGTCAGAAGGCAAAAAC |
| 235 | CCCAGCAGAGAATGGCTTGC |
| 236 | GTCATCAATTCCCCCCAGCA |
| 237 | TTATTACCCACCCAGGTAGC |
| 238 | GACTACTAGATCCCTGGATG |
| 239 | CATAATTGACTACTAGATCC |
| 240 | GGTCTATAGGCTGGAGGAGT |
| 241 | TTTGGTGGTCTATAGGCTGG |
| 242 | GATCTGCGTCTGCGAGGCGA |
| 243 | ATTAAAGATAGGTACTGTAG |
| 244 | AAAGGAAGGAGTTTGCCATT |
| 245 | AAATGAATCTTAGGAAAGGA |
| 246 | TTAATAATGTCCTCTTGTAA |
| 247 | AATATTTAGTGTGGGTAGGA |
| 248 | GTAATGATTAACTACCTGAT |
| 249 | TATAGAATACCAGCCTTCCA |
| 250 | CGTGTGGTTTCCCTCTTATA |
| 251 | ATGATCGGGAAAGAATCCCA |
| 252 | GGTCCAACTGATGATCGGGA |
| 253 | CTGGATTGTTTGAGTTGGCT |
| 254 | GATGGGGTTGAAGTCCCAAT |
| 255 | TGCTGGCCAGTGGTCCTTGA |
| 256 | GTGGAGGGGTGAACCCTGGC |
| 257 | CCCAAAACACCGCCGTGTGG |
| 258 | CGATTGGTGGAGGCAGGAGG |
| 259 | GAACATGGAGAACACCACAT |
| 260 | CTGTGTGTTTAATGACTGGG |
| 261 | TGGCCAGAGGCAAACCAGGT |
| 262 | GGGAGAGGCAGTAGTCGGAA |
| 263 | GGTGTTCTCCATGTTCGGTG |
| 264 | CTTGCGAAAGCCCAAGATGA |
| 265 | ACCTGACCGTTGCCGAGCAA |
| 266 | GAGCCCCAAACGGCCCCGAG |
| 267 | GGTCTGTAAGCAGGAGGAGT |
| 268 | TTTGGTGGTCTGTAAGCAGG |
| 269 | AAAAGAGGGAGTTTGCCACT |
| 270 | AAATGAATGTGAGGAAAAGA |
| 271 | TTAATAATGTCCTCCTGCAA |
| 272 | TATAAAATGCCCGCCTTCCA |
| 273 | CCTCTGCACGTCGCATGGAGAC |
| 274 | CCCACTGTCTGGCTTTCAGTTA |
| 275 | GAGTTGGCTTTGAATGCAGGGT |
| 276 | CCATGTTCGGTACAGGGTCCCC |
| 277 | GATACTGTTTACTTAGAAAGGC |
| 278 | CATTTCCTGTCTTACTTTTGGG |
| 279 | TGTTGACAAAAATCCTCACAAT |
| 280 | CATCTGCCGGACCGTGTGCACT |
| 281 | TCTGGACTATCAAGGTATGTTG |
| 282 | CATCCCATCATCTTGGGCTTTC |
| 283 | GCAATGTCAACGACCGACCTTG |
| 284 | GCAGTATGGATCGGCAGAGGAG |
| 285 | TGTGGCAATGTGCCCCAACTCC |
| 286 | CAGTCCCAAATCTCCAGTCACT |
| 287 | CACTCCTCCTGCATATAGACCA |
| 288 | TGTTGGTTCTTCTGGACTATCA |
| 289 | CACCTTATGTGTCCAAGGAATA |
| 290 | TCTGCGAGGCGAGGGAGTTCTT |
| 291 | GCGCCGACGGGACGTAAACAAA |
| 292 | CACCCAGGTGGCTAGATTCATC |
| 293 | TCTGCATCCTGCTGCTATGCCT |
| 294 | TCTACGGTACCTTGCTTTAATC |
| 295 | CAAAATACCTATGGGAGTGGGC |
| 296 | ATTCGAGATCTCCTCGACACCG |
| 297 | GGAACAGTGAGCCCTGCTCAGA |
| 298 | GCGACGCGGCGATTGAGACCTT |
| 299 | TGTCTTACTTTTGGGAGAGAAA |
| 300 | CCCCTCCCCATGGGGGACTGTT |
| 301 | CCCTAGAAAATTGAGAGAAGTC |
| 302 | CTTAACTTCATGGGATATGTAA |
| 303 | CTTCACCTCTGCACGTCGCATG |
| 304 | TGCTGGTGGCTCCAGTTCAGGA |
| 305 | CTCCCAAAAGTAAGACAGGAAA |
| 306 | CTCATGTTGCTGTACAAAACCT |
| 307 | CTTGGCTCAGTTTACTAGTGCC |
| 308 | CTCAATTTTCTAGGGGGAACAC |
| 309 | TGACTGCCGATTGGTGGAGGCA |
| 310 | CGGTGGTCTCCATGCGACGTGC |
| 311 | TGGGAACAAGATCTACAGCATG |
| 312 | TTTGTCTTTGGGTATACATTTA |
| 313 | CGCCAACTTACAAGGCCTTTCT |
| 314 | CGCAATGTGGATATCCTGCTTT |
| 315 | TGGGATATGTAATTGGGAGTTG |
| 316 | TGACATTCATTTGCAGGAGGAC |
| 317 | TGTAAACAGGCCTATTGATTGG |
| 318 | CGAGATTGAGATCTTCTGCGAC |
| 319 | TGAATATTTGGTGTCTTTTGGA |
| 320 | TGAACTGGAGCCACCAGCAGGA |
| 321 | GACTTCTTTCCTTCTATTCGAG |
| 322 | TCAACTCACCCCAACACAGAAT |
| 323 | CCTCACCATACGGCACTCAGGC |
| 324 | GAGCAGGGCTCACTGTTCCTGA |
| 325 | TGTCCTACTGTTCAAGCCTCCA |
| 326 | CCGCCTGTTGTACCGACCGACC |
| 327 | ATGGCTGCTAGGCTGTGCTGCC |
| 328 | GGAAGTGTTGATAAGATAGGGG |
| 329 | CAAGAATATGGTGACCCGCAAA |
| 330 | GTCCGTAGGTTTTGTACAGCAA |
| 331 | ACGCATGCGCTGATGGCCTATG |
| 332 | TTGGACACATAAGGTGGGAAAC |
| 333 | GTCCCCAATCCCCTGGGATTCT |
| 334 | GGAGACTCTAAGGCCTCCCGAT |
| 335 | ACCAAACTCTTCAAGATCCCAG |
| 336 | GTCGTGCTCTCCCGCAAGTATA |
| 337 | GTGGTTCGTAGGGCTTTCCCCC |
| 338 | GTGTTGGGGTGAGTTGATGAAT |
| 339 | AATCAATAGGCCTGTTTACAGG |
| 340 | AAGTAAACAGTATCTGAACCTT |
| 341 | GTACAGGGTCCCCAGTCTTCGA |
| 342 | GTTATATGGATGATGTGGTTTT |
| 343 | TCCCCGATCATCAGTTGGACCC |
| 344 | AAGACTGTGTGTTTACTGAGTG |
| 345 | TTTACTGTAAGGGGCCCCACAA |
| 346 | TTTCCTGACATTCATTTGCAGG |
| 347 | AAATTACTTCCCACCCAGGTGG |
| 348 | AAAGAGTGTGTAAATAATGTCT |
| 349 | TTTGCAGGAGGACATTGTTGAT |
| 350 | TAAAACACATTTTGATTTTTTG |
| 351 | AAACCTCGAAAAGGCATGGGGA |
| 352 | TAGGGCTTTCCCCCACTGTCTG |
| 353 | AAGCCAACTCAGAAAATCCAGA |
| 354 | ACTGCATGGCCTGAGGATGAGT |
| 355 | CTGGATGCTGGATCTTCCAAAT |
| 356 | AGGTTTGGAAGACCAACCTCCC |
| 357 | TCTAACAACAGTAGTTTCCGGA |
| 358 | TTCCTTCTATTCGAGATCTCCT |
| 359 | TTGACATACTTTCCAATCAATA |
| 360 | AGCCTCCAAGCTGTGCCTTGGG |
| 361 | TTCTATTCGAGATCTCCTCGAC |
| 362 | GGTGGGCGTTCACGGTGGTCTC |
| 363 | AGGATCATCAACAACCAGCACC |
| 364 | TTGAGCAGGAGTTGTGCAGGTT |
| 365 | AGATCTCCTCGACACCGCCTCT |
| 366 | AGATCCCAGAGTCAGGGCCCTG |
| 367 | ATAAGATTGACGATATGGCAGA |
| 368 | AGACGAGACATTATTTACACAC |
| 369 | AGAACAGTTTCTCTCCCAAAAG |
| 370 | GGCCAGGGTTCACCCCTCCCCA |
| 371 | AGGGGGAACACCCGTGTGTCTT |
| 372 | GGAACAGTAAACCCTGTTCCGA |
| 373 | TAGGACCCCTGCTCGTGTTACA |
| 374 | CTTCCAAAAGTAAGACAGGAAA |
| 375 | GGCCAGGGTTCACCCCACCACA |
| 376 | GGATAATAAGGTTTAATGCCTT |
| 377 | TAGGGCTTTCCCCCACTGTTTG |
| 378 | TGAGTATTTGGTGTCTTTTGGA |
| 379 | TCCCCATGCCTTGTCGAGGTTT |
| 380 | TATGGGAGTGGGCCTCAGTCCG |
| 381 | TCATCTGCCGTTCCGGCCGACC |
| 382 | CTTTCTCGCCAACTTACAAGGC |
| 383 | CTGGATGCTGGGTCTTCCAAAT |
| 384 | GATATTGTTTACACAGAAAGGC |
| 385 | TCTACTGTACCTGTCTTTAATC |
| 386 | TCCTGCTGCTATGCCTCATCTT |
| 387 | GATAAGTTTCGCTCCAGACCGG |
| 388 | GTCCGAAGGTTTTGTACAGCAA |
| 389 | GAGCCAACTCAAACAATCCAGA |
| 390 | TCTTCATCCTGCTGCTATGCCT |
| 391 | GTGCAGGGTCCCCAGTCCTCGA |
| 392 | GTTCCCAATCCTCTGGGATTCT |
| 393 | TCTGGACTACCAAGGTATGTTG |
| 394 | GAGAGAGGACAACAGAGTTGTC |
| 395 | GTTATATGGATGATGTGGTATT |
| 396 | GGCCGACCACGGGGCGCACCTC |
| 397 | GACTACTGCCTCACCCATATCG |
| 398 | GCGCCGACGGGACGTAGACAAA |
| 399 | GACGGAAACTGCACTTGTATTC |
| 400 | GTGTAAACAATATCTGAACCTT |
| 401 | GGAACTGGAGCCACCAGCAGGA |
| 402 | TTTGTCTTTGGGTATACATTTG |
| 403 | CTCTTGTTGCTGTACAAAACCT |
| 404 | CAATCCTCTGGGATTCTTTCCC |
| 405 | CAAGATTCCTATGGGAGTGGGC |
| 406 | CAAGAATATGGTGACCCACAAA |
| 407 | TGCTCAAGGAACCTCTATGTTT |
| 408 | TTATACGGGTCAATGTCCATGC |
| 409 | TTCCCGATCACCAGTTGGACCC |
| 410 | ATCCTAACCTTACCAAATATTT |
| 411 | ATATAAGAGAGAAACTACACGC |
| 412 | AGGGGGAGCACCCACGTGTCCT |
| 413 | TTGAGCAGGAATCGTGCAGGTC |
| 414 | ACTGCATGGCCTGAGGATGACT |
| 415 | ACGCATGCGCCGATGGCCTATG |
| 416 | ACCCCAACAAGGATCACTGGCC |
| 417 | ACCAAGCTCTGCTAGATCCCAG |
| 418 | ACAGAGTATGTAAATAATGCCT |
| 419 | AATTACATATCCCATGAAGTTA |
| 420 | AATGTATACCCAAAGACAAAAG |
| 421 | AATCAATAGGTCTATTTACAGG |
| 422 | TTTACAGGAGGACATTATTAAT |
| 423 | AAGACTGTTTGTTTAAAGACTG |
| 424 | AAACTAGGCATTATTTACATAC |
| 425 | AAACCTCGACAAGGCATGGGGA |
| 426 | AAAAGTAAGACAGGAAATGTGA |
| 427 | AAAACTGCCTGTAAATAGACCT |
| 428 | AAAACATTGCTTGATTTTTAGT |
| 429 | CACCCAGGTGGCCAGATTCATC |
| 430 | TTAACTGTAAGAGGGCCCACAT |
| 431 | ATGGCTGCTAGGGTGTGCTGCC |
| 432 | CACTCCTCCCGCTTACAGACCA |
| 433 | CTCTTATATAGAATGCCAGCCT |
| 434 | TGCTGGTGGCTCCAGTTCCGGA |
| 435 | CTCCAGACCGGCTGCGAGCAAA |
| 436 | TGGAAGTAGAGGACAAACGGGC |
| 437 | CTCAATTTTCTAGGGGGAGCAC |
| 438 | TGGGAACAAGAGCTACAGCATG |
| 439 | TGGGATATGTAATTGGAAGTTG |
| 440 | CGATCACCAGTTGGACCCTGCG |
| 441 | CGAGGACTGGGGACCCTGCACC |
| 442 | CCTGGCTCAGTTTACTAGTGCC |
| 443 | CACCTTATGAGTCCAAGGGATA |
| 444 | CCTCTGCCTAATCATCTCATGT |
| 445 | TGTCAACAAGAAAAACCCCGCC |
| 446 | CCTCACCATACAGCACTCAGGC |
| 447 | TGTCTTACTTTTGGAAGAGAAA |
| 448 | TCACATTCATTTACAGGAGGAC |
| 449 | TGTTGGTTCTTCTGGACTACCA |
| 450 | CAGTCCCCAACCTCCAATCACT |
| 451 | CATCCCATCATCCTGGGCTTTC |
| 452 | TGTGGTAAAGTACCCCAACTTC |
| 453 | TGTTGACAAGAATCCTCACAAT |
| 454 | CCATGTTCGGTGCAGGGTCCCC |
| 455 | CCCACTGTTTGGCTTTCAGTTA |
| 456 | CATTTCCTGTCTTACTTTTGGA |
| 457 | TCTGGATTATCAAGGTATGTTG |
| 458 | TTTGTCTCTGGGTATACATTTA |
| 459 | TTTGTCTAAGGGCAAATATTTA |
| 460 | TCCCCATGCCTTTGCGAGGTTT |
| 461 | TTATAAGGGTCAATGTCCATGC |
| 462 | TTTCCTAAGATTCATTTACAAG |
| 463 | TTTACAGTGAGAGGGCCCACAA |
| 464 | TGGGTTACATAATTGGAAGTTG |
| 465 | TTCCCGATCATCAGTTGGACCC |
| 466 | TTTACAAGAGGACATTATTAAT |
| 467 | TCCTCCTGCCTCCACCAATCGG |
| 468 | TTGGACTCATAAGGTGGGAAAC |
| 469 | TCTACAGTACCTATCTTTAATC |
| 470 | TTCCGTCAGAGATCTCCTAGAC |
| 471 | TTCCTTCCGTCAGAGATCTCCT |
| 472 | TGTTAACAGGCCTATTGATTGG |
| 473 | TGTACTGTTTACTTAGAAAGGC |
| 474 | AAAACAGTGTTTGATCTTTTGT |
| 475 | CCGTCTGCCGTTCCAGCCGACC |
| 476 | AAGACTGTGTGTTTAAGGACTG |
| 477 | CCTCTGCCTAATCATCTCTTGT |
| 478 | CCTCTGCACGTTGCATGGAGAC |
| 479 | TCACAGGGTCCCCAGTCCTCGC |
| 480 | CCCCTCCACACGGCGGTGTTTT |
| 481 | CCCCAGCAGAGAATGGCTTGCC |
| 482 | CCCACTGTTTGGCTTTCAGCTA |
| 483 | CCATGTTCGTCACAGGGTCCCC |
| 484 | CCAACTTCCAATTATGTAACCC |
| 485 | CATCTGCCGGTCCGTGTGCACT |
| 486 | CATCCCATCGTCCTGGGCTTTC |
| 487 | CACTCCTCCAGCCTATAGACCA |
| 488 | CACCTTATGAGTCCAAGGAATA |
| 489 | CAACCCTCTGGGATTCTTTCCC |
| 490 | ATGGCTGCTAGGCTGTACTGCC |
| 491 | ATCTTCTCTTTTCATTTACAGT |
| 492 | ATCCTACCCACACTAAATATTT |
| 493 | ATCCGTAGGTTTTGTACAGCAA |
| 494 | ATATAAGAGGGAAACCACACGT |
| 495 | AGTACAGTCTCTCTTCCAAAAG |
| 496 | AGGGGGATCACCCGTGTGTCTT |
| 497 | AGGATCAACAACAACCAGTACG |
| 498 | AGCCGACCACGGGGCGCACCTC |
| 499 | AAGTAAACAGTACATGAACCTT |
| 500 | ACTGCCTTCCACCAAGCTCTGC |
| 501 | ACCCCATCAAGGACCACTGGCC |
| 502 | ACCAAGCTCTGCAGGATCCCAG |
| 503 | CGATACAGAGCTGAGGCGGTGT |
| 504 | AATCAATAGGCCTGTTAACAGG |
| 505 | AAATTATTACCCACCCAGGTAG |
| 506 | CGGGACGTCCTTTGTTTACGTC |
| 507 | TATTGGTTCTTCTGGATTATCA |
| 508 | TATATCTTGCCTTACTTTTGGA |
| 509 | TAAGATTCATTTACAAGAGGAC |
| 510 | GTTCCCAACCCTCTGGGATTCT |
| 511 | GTCGTCCTCTCGCGGAAATATA |
| 512 | GTCAGAGATCTCCTAGACACCG |
| 513 | AAAAGTAAGGCAAGATATATGA |
| 514 | GGCCAGGGTTCACCCCTCCACA |
| 515 | GGATTAAAGATAGGTACTGTAG |
| 516 | GGAACAGTAAACCCTGCTCCGA |
| 517 | GGAAACTACTGTTGTTAGACGA |
| 518 | GCTATATGGATGATGTGGTATT |
| 519 | GCGAGAGGACGACAGAATTGTC |
| 520 | GCGACGCGGCGATTGAGATCTG |
| 521 | GATGAGCTTTGCTCCAGACCGG |
| 522 | GAGTGTGGATTCGCACTCCTCC |
| 23 | GAGCAGGGTTTACTGTTCCTGA |
| 524 | GACTTCTTTCCTTCCGTCAGAG |
| 525 | AAACCAGACATTATTTACATAC |
| 526 | AAAGAGTATGTAAATAATGTCT |
| 527 | CTTTTCATTTACAGTGAGAGGG |
| 528 | CTTCACCTCTGCACGTTGCATG |
| 529 | CTGCTGGGGGGAATTGATGACT |
| 530 | CTCTTATATAGAATACCAGCCT |
| 531 | CTCAATTTTCTAGGGGGATCAC |
| 532 | CTAAACTTCATGGGTTACATAA |
| 533 | CGGTGGTCTCCATGCAACGTGC |
| 534 | CGATCATCAGTTGGACCCTGCA |
| 535 | AATTATGTAACCCATGAAGTTT |
| 536 | AAGACTGTGTGTTTAATGACTG |
| 537 | TTTGCAGGAGGACATTATTAAT |
| 538 | TCACATTCATTTGCAGGAGGAC |
| 539 | TTGAGCAGGAGTCGTGCAGGTC |
| 540 | CACTCCTCCTGCTTACAGACCA |
| 541 | TGTAAATAGACCTATTGATTGG |
| 542 | CGATCACCAGTTGGACCCTGCA |
| 543 | CTCTTATATAAAATGCCCGCCT |
| 544 | GAAACTTCCTGTAAATAGACCT |
| 545 | GACTACTGCCTCTCCCATATCG |
| 546 | TCGTCTGCCGTTCCGGCCGACC |
| 547 | TTGTACTAGGAGGCTGTAGGCA |
| 548 | GAAAACATTGTTTGATTTTTTG |
| 549 | CCTCTGCACGTCGCATGGAGAC |

In some embodiments, the target nucleic acid sequence comprises about 15 nucleotides to about 28 nucleotides within a sequence, the sequence comprising at least about 70%, 75%, 80%, 85%, 90%, or 95% sequence identity to any one of SEQ ID NOs: 2-5. In some embodiments, the target nucleic acid sequence comprises at least about 15 nucleotides within a sequence, the sequence comprising at least about 70%, 75%, 80%, 85%, 90%, or 95% sequence identity to any one of SEQ ID NOs: 2-5. In some embodiments, the target nucleic acid sequence comprises at most about 28 nucleotides within a sequence, the sequence comprising at least about 70%, 75%, 80%, 85%, 90%, or 95% sequence identity to any one of SEQ ID NOs: 2-5. In some embodiments, the target nucleic acid sequence comprises about 15 nucleotides to about 16 nucleotides, about 15 nucleotides to about 17 nucleotides, about 15 nucleotides to about 18 nucleotides, about 15 nucleotides to about 19 nucleotides, about 15 nucleotides to about 20 nucleotides, about 15 nucleotides to about 21 nucleotides, about 15 nucleotides to about 22 nucleotides, about 15 nucleotides to about 23 nucleotides, about 15 nucleotides to about 24 nucleotides, about 15 nucleotides to about 25 nucleotides, about 15 nucleotides to about 28 nucleotides, about 16 nucleotides to about 17 nucleotides, about 16 nucleotides to about 18 nucleotides, about 16 nucleotides to about 19 nucleotides, about 16 nucleotides to about 20 nucleotides, about 16 nucleotides to about 21 nucleotides, about 16 nucleotides to about 22 nucleotides, about 16 nucleotides to about 23 nucleotides, about 16 nucleotides to about 24 nucleotides, about 16 nucleotides to about 25 nucleotides, about 16 nucleotides to about 28 nucleotides, about 17 nucleotides to about 18 nucleotides, about 17 nucleotides to about 19 nucleotides, about 17 nucleotides to about 20 nucleotides, about 17 nucleotides to about 21 nucleotides, about 17 nucleotides to about 22 nucleotides, about 17 nucleotides to about 23 nucleotides, about 17 nucleotides to about 24 nucleotides, about 17 nucleotides to about 25 nucleotides, about 17 nucleotides to about 28 nucleotides, about 18 nucleotides to about 19 nucleotides, about 18 nucleotides to about 20 nucleotides, about 18 nucleotides to about 21 nucleotides, about 18 nucleotides to about 22 nucleotides, about 18 nucleotides to about 23 nucleotides, about 18 nucleotides to about 24 nucleotides, about 18 nucleotides to about 25 nucleotides, about 18 nucleotides to about 28 nucleotides, about 19 nucleotides to about 20 nucleotides, about 19 nucleotides to about 21 nucleotides, about 19 nucleotides to about 22 nucleotides, about 19 nucleotides to about 23 nucleotides, about 19 nucleotides to about 24 nucleotides, about 19 nucleotides to about 25 nucleotides, about 19 nucleotides to about 28 nucleotides, about 20 nucleotides to about 21 nucleotides, about 20 nucleotides to about 22 nucleotides, about 20 nucleotides to about 23 nucleotides, about 20 nucleotides to about 24 nucleotides, about 20 nucleotides to about 25 nucleotides, about 20 nucleotides to about 28 nucleotides, about 21 nucleotides to about 22 nucleotides, about 21 nucleotides to about 23 nucleotides, about 21 nucleotides to about 24 nucleotides, about 21 nucleotides to about 25 nucleotides, about 21 nucleotides to about 28 nucleotides, about 22 nucleotides to about 23 nucleotides, about 22 nucleotides to about 24 nucleotides, about 22 nucleotides to about 25 nucleotides, about 22 nucleotides to about 28 nucleotides, about 23 nucleotides to about 24 nucleotides, about 23 nucleotides to about 25 nucleotides, about 23 nucleotides to about 28 nucleotides, about 24 nucleotides to about 25 nucleotides, about 24 nucleotides to about 28 nucleotides, or about 25 nucleotides to about 28 nucleotides within a sequence, the sequence comprising at least about 70%, 75%, 80%, 85%, 90%, or 95% sequence identity to any one of SEQ ID NOs: 2-5. In some embodiments, the target nucleic acid sequence comprises about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, or about 28 nucleotides within a sequence, the sequence comprising at least about 70%, 75%, 80%, 85%, 90%, or 95% sequence identity to any one of SEQ ID NOs: 2-5.

In some embodiments, the target nucleic acid sequence is a naturally occurring sequence. In other embodiments, the target nucleic acid sequence is a non-naturally occurring sequence.

Generally, a target sequence comprises a protospacer adjacent motif (PAM). In some instances, a PAM refers to a DNA sequence required for a Cas/sgRNA to form an R-loop to interrogate a specific DNA sequence through Watson-Crick pairing of its guide RNA with the genome. In certain instances, the PAM specificity is a function of the DNA-binding specificity of the Cas protein (e.g., a PAM recognition domain of a Cas), wherein, a protospacer adjacent motif recognition domain, in some instances, refers to a Cas amino acid sequence that comprises a binding site to a DNA target PAM sequence. In the CRISPR-Cas system derived from S. pyogenes (spCas9), the target DNA typically immediately precedes a 5′-NGG or NAG proto-spacer adjacent motif (PAM). Other Cas9 orthologs can have different PAM specificities. For example, Cas9 from S. thermophilus (stCas9) requires 5′-NNAGAA for CRISPR 1 and 5′-NGGNG for CRISPR3 and Neiseria menigiditis (nmCas9) requires 5′-NNNNGATT. Cas9 from Staphylococcus aureus subsp. aureus (saCas9) requires 5′-NNGRRT (R=A or G). The HBV targets provided herein can further be identified in the context of the gRNAs. CasX (e.g. CasX from deltaporoteobacteria or planctomycetes) recognizes a TTCN PAM element.

The gRNA is a short RNA nucleotide spacer that defines the genomic target to be modified. As used herein, the term guide RNA (gRNA or sgRNA) refers to a RNA containing a sequence that corresponds and/or hybridizes to a target HBV sequence. The guide RNA sequence can be a sense or anti-sense sequence. A guide RNA can, in some embodiments, include nucleotide sequences other than the region complementary or substantially complementary to a region of a target DNA sequence. For example, in some instances, a guide RNA is part or considered part of a crRNA or an included in a crRNA, e.g., a crRNA: tracrRNA chimera. As used herein, a term target nucleic acid is intended to mean a nucleic acid that is the object of an action (e.g. editing or modulation).

In some embodiments, the gRNA is a synthetic oligonucleotide. In some embodiments, the synthetic nucleotide comprises a modified nucleotide. Modification of the inter-nucleoside linker (i.e. backbone) can be utilized to increase stability or pharmacodynamic properties. For example, inter-nucleoside linker modifications prevent or reduce degradation by cellular nucleases, thus increasing the pharmacokinetics and bioavailability of the gRNA. Generally, a modified inter-nucleoside linker includes any linker other than other than phosphodiester (PO) liners, that covalently couples two nucleosides together. In some embodiments, the modified inter-nucleoside linker increases the nuclease resistance of the gRNA compared to a phosphodiester linker. For naturally occurring oligonucleotides, the inter-nucleoside linker includes phosphate groups creating a phosphodiester bond between adjacent nucleosides. In some embodiments, the gRNA comprises one or more inter-nucleoside linkers modified from the natural phosphodiester. In some embodiments, all of the inter-nucleoside linkers of the gRNA, or contiguous nucleotide sequence thereof, are modified. For example, in some embodiments the inter-nucleoside linkage comprises Sulphur (S), such as a phosphorothioate inter-nucleoside linkage.

Modifications to the ribose sugar or nucleobase can also be utilized herein. Generally, a modified nucleoside includes the introduction of one or more modifications of the sugar moiety or the nucleobase moiety. In some embodiments, the gRNAs, as described, comprise one or more nucleosides comprising a modified sugar moiety, wherein the modified sugar moiety is a modification of the sugar moiety when compared to the ribose sugar moiety found in deoxyribose nucleic acid (DNA) and RNA. Numerous nucleosides with modification of the ribose sugar moiety can be utilized, primarily with the aim of improving certain properties of oligonucleotides, such as affinity and/or stability. Such modifications include those where the ribose ring structure is modified. These modifications include replacement with a hexose ring (HNA), a bicyclic ring having a biradical bridge between the C2 and C4 carbons on the ribose ring (e.g. locked nucleic acids (LNA)), or an unlinked ribose ring which typically lacks a bond between the C2 and C3 carbons (e.g. UNA). Other sugar modified nucleosides include, for example, bicyclohexose nucleic acids or tricyclic nucleic acids. Modified nucleosides also include nucleosides where the sugar moiety is replaced with a non-sugar moiety, for example in the case of peptide nucleic acids (PNA), or morpholino nucleic acids.

Sugar modifications also include modifications made by altering the substituent groups on the ribose ring to groups other than hydrogen, or the 2′-OH group naturally found in DNA and RNA nucleosides. Substituents may, for example be introduced at the 2′, 3′, 4′ or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity. A 2′ sugar modified nucleoside is a nucleoside that has a substituent other than H or —OH at the 2′ position (2′ substituted nucleoside) or comprises a 2′ linked biradicle, and includes 2′ substituted nucleosides and LNA (2′-4′ biradicle bridged) nucleosides. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. By way of further example, in some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the gRNA comprises one or more modified sugars. In some embodiments, the gRNA comprises only modified sugars. In certain embodiments, the gRNA comprises greater than 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the gRNA comprises both inter-nucleoside linker modifications and nucleoside modifications.

Target specificity can be used in reference to a guide RNA, or a crRNA specific to a target polynucleotide sequence or region and further includes a sequence of nucleotides capable of selectively annealing/hybridizing to a target (sequence or region) of a target polynucleotide (e.g. corresponding to a target), e.g., a target DNA. In some embodiments, a crRNA or the derivative thereof contains a target-specific nucleotide region complementary to a region of the target DNA sequence. In some embodiments, a crRNA or the derivative thereof contains other nucleotide sequences besides a target-specific nucleotide region. In some embodiments, the other nucleotide sequences are from a tracrRNA sequence.

gRNAs are generally supported by a scaffold, wherein a scaffold refers to the portions of gRNA or crRNA molecules comprising sequences which are substantially identical or are highly conserved across natural biological species (e.g. not conferring target specificity). Scaffolds include the tracrRNA segment and the portion of the crRNA segment other than the polynucleotide-targeting guide sequence at or near 5′ end of the crRNA segment, excluding any unnatural portions comprising sequences not conserved in native crRNAs and tracrRNAs. In some embodiments, the crRNA or tracrRNA comprises a modified sequence. In certain embodiments, the crRNA or tracrRNA comprises at least 1, 2, 3, 4, 5, 10, or 15 modified bases (e.g. a modified native base sequence).

“Complementary,” as used herein, generally refers to a polynucleotide that includes a nucleotide sequence capable of selectively annealing to an identifying region of a target polynucleotide under certain conditions. As used herein, the term “substantially complementary” and grammatical equivalents is intended to mean a polynucleotide that includes a nucleotide sequence capable of specifically annealing to an identifying region of a target polynucleotide under certain conditions. Annealing refers to the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In some embodiments, base-stacking and hydrophobic interactions can also contribute to duplex stability. Conditions under which a polynucleotide anneals to complementary or substantially complementary regions of target nucleic acids are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968). Annealing conditions will depend upon the particular application and can be routinely determined by persons skilled in the art, without undue experimentation. Hybridization generally refers to process in which two single-stranded polynucleotides bind non-covalently to form a stable double-stranded polynucleotide. A resulting double-stranded polynucleotide is a “hybrid” or “duplex.” In certain instances, 100% sequence identity is not required for hybridization and, in certain embodiments, hybridization occurs at about greater than 70%, 75%, 80%, 85%, 90%, or 95% sequence identity. In certain embodiments, sequence identity includes in addition to non-identical nucleobases, sequences comprising insertions and/or deletions.

In some embodiments, the gRNA comprises a CRISPR RNA (crRNA): trans activating cRNA (tracrRNA) duplex. In some embodiments, the gRNA comprises a stem-loop that mimics the natural duplex between the crRNA and tracrRNA. In some embodiments, the stem-loop comprises a nucleotide sequence comprising AGAAAU. For example, in some embodiments, the composition comprises a synthetic or chimeric guide RNA comprising a crRNA, stem, and tracrRNA. In some embodiments, the composition comprises an isolated crRNA and/or an isolated tracrRNA which hybridize to form a natural duplex. For example, in some embodiments, the gRNA comprises a crRNA or crRNA precursor (pre-crRNA) comprising a targeting sequence.

In some embodiments, the gRNA comprises about 15 nucleotides to about 28 nucleotides. In some embodiments, the gRNA comprises at least about 15 nucleotides. In some embodiments, the gRNA comprises at most about 28 nucleotides. In some embodiments, the gRNA comprises about 15 nucleotides to about 16 nucleotides, about 15 nucleotides to about 17 nucleotides, about 15 nucleotides to about 18 nucleotides, about 15 nucleotides to about 19 nucleotides, about 15 nucleotides to about 20 nucleotides, about 15 nucleotides to about 21 nucleotides, about 15 nucleotides to about 22 nucleotides, about 15 nucleotides to about 23 nucleotides, about 15 nucleotides to about 24 nucleotides, about 15 nucleotides to about 25 nucleotides, about 15 nucleotides to about 28 nucleotides, about 16 nucleotides to about 17 nucleotides, about 16 nucleotides to about 18 nucleotides, about 16 nucleotides to about 19 nucleotides, about 16 nucleotides to about 20 nucleotides, about 16 nucleotides to about 21 nucleotides, about 16 nucleotides to about 22 nucleotides, about 16 nucleotides to about 23 nucleotides, about 16 nucleotides to about 24 nucleotides, about 16 nucleotides to about 25 nucleotides, about 16 nucleotides to about 28 nucleotides, about 17 nucleotides to about 18 nucleotides, about 17 nucleotides to about 19 nucleotides, about 17 nucleotides to about 20 nucleotides, about 17 nucleotides to about 21 nucleotides, about 17 nucleotides to about 22 nucleotides, about 17 nucleotides to about 23 nucleotides, about 17 nucleotides to about 24 nucleotides, about 17 nucleotides to about 25 nucleotides, about 17 nucleotides to about 28 nucleotides, about 18 nucleotides to about 19 nucleotides, about 18 nucleotides to about 20 nucleotides, about 18 nucleotides to about 21 nucleotides, about 18 nucleotides to about 22 nucleotides, about 18 nucleotides to about 23 nucleotides, about 18 nucleotides to about 24 nucleotides, about 18 nucleotides to about 25 nucleotides, about 18 nucleotides to about 28 nucleotides, about 19 nucleotides to about 20 nucleotides, about 19 nucleotides to about 21 nucleotides, about 19 nucleotides to about 22 nucleotides, about 19 nucleotides to about 23 nucleotides, about 19 nucleotides to about 24 nucleotides, about 19 nucleotides to about 25 nucleotides, about 19 nucleotides to about 28 nucleotides, about 20 nucleotides to about 21 nucleotides, about 20 nucleotides to about 22 nucleotides, about 20 nucleotides to about 23 nucleotides, about 20 nucleotides to about 24 nucleotides, about 20 nucleotides to about 25 nucleotides, about 20 nucleotides to about 28 nucleotides, about 21 nucleotides to about 22 nucleotides, about 21 nucleotides to about 23 nucleotides, about 21 nucleotides to about 24 nucleotides, about 21 nucleotides to about 25 nucleotides, about 21 nucleotides to about 28 nucleotides, about 22 nucleotides to about 23 nucleotides, about 22 nucleotides to about 24 nucleotides, about 22 nucleotides to about 25 nucleotides, about 22 nucleotides to about 28 nucleotides, about 23 nucleotides to about 24 nucleotides, about 23 nucleotides to about 25 nucleotides, about 23 nucleotides to about 28 nucleotides, about 24 nucleotides to about 25 nucleotides, about 24 nucleotides to about 28 nucleotides, or about 25 nucleotides to about 28 nucleotides. In some embodiments, the gRNA comprises about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, or about 28 nucleotides.

Described herein are gRNAs targeting (e.g. hybridizing or annealing to) a region within a HBV genome. In some embodiments, a gRNA is encoded by a sequence having at least 90%, 95%, 97%, 98%, or 99% sequence identity to any one of SEQ ID NOs: 6-549. In some embodiments, a gRNA is encoded by a sequence according to any one of SEQ ID NOs: 6-272. In some embodiments, a gRNA is encoded by a sequence according to any one of SEQ ID NOs: 6-272 comprising 1, 2, or 3 modifications. In some embodiments, the modification is a substitution, deletion, insertion, or a combination thereof.

Described herein are gRNAs targeting (e.g. hybridizing or annealing to) a region within a HBV genome. In some embodiments, a gRNA is encoded by a sequence having at least 90%, 95%, 97%, 98%, or 99% sequence identity to any one of SEQ ID NOs: 6-272. In some embodiments, a gRNA is encoded by a sequence according to any one of SEQ ID NOs: 6-272. In some embodiments, a gRNA is encoded by a sequence according to any one of SEQ ID NOs: 6-272 comprising 1, 2, or 3 modifications. In some embodiments, the modification is a substitution, deletion, insertion, or a combination thereof.

Described herein are gRNAs targeting (e.g. hybridizing or annealing to) a region within a HBV genome. In some embodiments, a gRNA is encoded by a sequence having at least 90%, 95%, 97%, 98%, or 99% sequence identity to any one of SEQ ID NOs: 273-548. In some embodiments, a gRNA is encoded by a sequence according to any one of SEQ ID NOs: 6-272. In some embodiments, a gRNA is encoded by a sequence according to any one of SEQ ID NOs: 6-272 comprising 1, 2, or 3 modifications. In some embodiments, the modification is a substitution, deletion, insertion, or a combination thereof.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

The term “homology” or “similarity” between two proteins is determined by comparing the amino acid sequence and its conserved amino acid substitutes of one protein sequence to the second protein sequence. Similarity can be determined by procedures which are well-known in the art, for example, a BLAST program (Basic Local Alignment Search Tool at the National Center for Biological Information).

In some embodiments, the CRISPR-associated endonuclease is Type I, Type II, or Type III Cas endonuclease. In other embodiments, the CRISPR-associated endonuclease is Cas3, Cas4, Cas5, Cas5e (or CasD), Cas6, Case, Cas6f, Cas7, Cas8al, Cas8a2, Cas8b, Cas8c, Cas9, Cas10, Cas10d, CasF, CasG, CasH, CasX, CasΦ, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. By way of further example, in some embodiments, the CRISPR-Cas protein is a Cas1, Cas1B, Cas2, Cas3, Cas4, Cas5, Cash, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx16, CsaX, Csx3, Csx1, Csx15, Csf1, Csf2, Csf3, Csf4, Cas9, Cas12 (e.g., Cas12a, Cas12b, Cas12c, Cas12d, Cas12k, Cas12j/CasΦ, Cas12L etc.), Cas13 (e.g., Cas13a, Cas13b (such as Cas13b-t1, Cas13b-t2, Cas13b-t3), Cas13c, Cas13d, etc.), Cas14, CasX, CasY, or an engineered form of the Cas protein. In some embodiments, the CRISPR/Cas protein or endonuclease is Cas9. In some embodiments, the CRISPR/Cas protein or endonuclease is Cas12. In certain embodiments, the Cas12 polypeptide is Cas12a, Cas12b, Cas12c, Cas12d, Cas12e, Cas12g, Cas12h, Cas12i, Cas12L or Cas12J. In some embodiments, the CRISPR/Cas protein or endonuclease is CasX. In some embodiments, the CRISPR/Cas protein or endonuclease is CasY. In some embodiments, the CRISPR/Cas protein or endonuclease is CasΦ. In other embodiments, the CRISPR-associated endonuclease is a Cas9 endonuclease. In other embodiments, the Cas9 endonuclease is a Staphylococcus aureus Cas9 endonuclease. In other embodiments, the Cas9 endonuclease is a Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Fine goldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina Cas9 endonuclease. In some embodiments, the CRISPR-associated endonuclease is a CasX endonuclease. In some embodiments, the CasX endonuclease is a deltaproteobacteria CasX or a planctomycetes Cas X endonuclease. In some embodiments, the CasX endonuclease is a deltaproteobacteria CasX. In some embodiments, the CasX endonuclease is a planctomycetes Cas X endonuclease.

Provided herein is a nucleic acid encoding the CRISPR-Cas systems described herein. In some embodiments, the nucleic acid further comprises a 5′ ITR element and 3′ ITR element. In some embodiments, the nucleic acid is configured to be packaged into an adeno-associated virus (AAV) vector. In some embodiments, the adeno-associated virus (AAV) vector is AAV2, AAV5, AAV6, AAV7, AAV8, or AAV9. In some embodiments, the adeno-associated virus (AAV) vector is AAV1, AAV2, AAV3, AAV4, AAV5, AAV6, AAV7, AAV8, AAV9, AAV10, AAV11, AAVDJ, or AAVDJ/8.

Further provided are adeno-associated virus (AAV) vectors comprising a nucleic acid described herein. In some embodiments, the CRISPR-associated endonuclease is a Cas9 endonuclease, a Cas12 endonuclease, a CasX endonuclease, or a CasΦ endonuclease. In some embodiments, the CRISPR-associated endonuclease is a Cas9 endonuclease. In some embodiments, the Cas9 endonuclease is a Staphylococcus aureus Cas9 endonuclease. In some embodiments, the CRISPR-associated endonuclease is a CasX endonuclease. In some embodiments, the CasX endonuclease is a deltaproteobacteria CasX or a planctomycetes Cas X endonuclease. In some embodiments, the CasX endonuclease is a deltaproteobacteria CasX. In some embodiments, the CasX endonuclease is a planctomycetes Cas X endonuclease. In some embodiments, the AAV vector is an AAV6 vector or an AAV9 vector. In some embodiments, the adeno-associated virus (AAV) vector is AAV1, AAV2, AAV3, AAV4, AAV5, AAV6, AAV7, AAV8, AAV9, AAV10, AAV11, AAVDJ, or AAVDJ/8.

### Vectors

The present disclosure includes a vector comprising one or more cassettes for expression of CRISPR components such as one or more gRNAs and a Cas endonuclease. Described herein, in some embodiments, are vectors comprising a nucleic acid encoding: (a) a Clustered Regularly Interspaced Short Palindromic Repeat (CRISPR)-associated endonuclease; and (b) one or more guide RNAs (gRNAs) or a nucleic acid sequence encoding the one or more gRNAs, the one or more gRNA hybridizes or is complementary to a target nucleic acid sequence within a Hepatitis B Virus (HBV) genome, the HBV genome comprising at least about 90% sequence identity to any one of SEQ ID NOs: 2-5. In some embodiments, the HBV genome comprises a sequence of any one of SEQ ID NOs: 2-5. In some embodiments, a gRNA of the one or more gRNAs is encoded by a sequence having at least 90%, 95%, 97%, 98%, or 99% sequence identity to any one of SEQ ID NOs: 6-549. In some embodiments, a gRNA of the one or more gRNAs is encoded by a sequence according to any one of SEQ ID NOs: 6-549. In some embodiments, a gRNA of the one or more gRNAs is encoded by a sequence according to any one of SEQ ID NOs: 6-549 comprising 1, 2, or 3 modifications. In some embodiments, a gRNA of the one or more gRNAs is encoded by a sequence having at least 90%, 95%, 97%, 98%, or 99% sequence identity to any one of SEQ ID NOs: 6-272. In some embodiments, a gRNA of the one or more gRNAs is encoded by a sequence according to any one of SEQ ID NOs: 6-272. In some embodiments, a gRNA of the one or more gRNAs is encoded by a sequence according to any one of SEQ ID NOs: 6-272 comprising 1, 2, or 3 modifications. In some embodiments, a gRNA of the one or more gRNAs is encoded by a sequence having at least 90%, 95%, 97%, 98%, or 99% sequence identity to any one of SEQ ID NOs: 273-578. In some embodiments, a gRNA of the one or more gRNAs is encoded by a sequence according to any one of SEQ ID NOs: 273-548. In some embodiments, a gRNA of the one or more gRNAs is encoded by a sequence according to any one of SEQ ID NOs: 273-548 comprising 1, 2, or 3 modifications. In some embodiments, the modification is a substitution, deletion, insertion, or a combination thereof. In some embodiments, the target nucleic acid sequence is located within a structural gene, non-structural gene, or combinations thereof. In some embodiments, the target nucleic acid sequence is located within a C, X, P, or S region.

In some embodiments, the CRISPR-associated endonuclease is Type I, Type II, or Type III Cas endonuclease. In some embodiments, the CRISPR-associated endonuclease is a Cas9 endonuclease, a Cas12 endonuclease, a CasX endonuclease, or a CasΦ endonuclease. In some embodiments, the CRISPR-associated endonuclease is a Cas9 endonuclease. In some embodiments, the Cas9 endonuclease is a Staphylococcus aureus Cas9 endonuclease. In some embodiments, the CRISPR-associated endonuclease is a CasX endonuclease. In some embodiments, the CasX endonuclease is a deltaproteobacteria CasX or a planctomycetes Cas X endonuclease. In some embodiments, the CasX endonuclease is a deltaproteobacteria CasX. In some embodiments, the CasX endonuclease is a planctomycetes Cas X endonuclease.

In some embodiments, the HBV is HBV-A genotype. In some embodiments, the HBV is HBV-B genotype. In some embodiments, the HBV is HBV-C genotype. In some embodiments, the HBV is HBV-A, HBV-B, HBV-C, HBV-D, HBV-E, HBV-F, HBV-G, or HBV-H genotype. In some embodiments, the HBV is HBV-A1, HBV-A2, HBV-QS-A3, or HBV-A4 genotype. In some embodiments, the HBV is HBV-B1, HBV-B2, HBV-QS-B3, HBV-B4, or HBV-B5 genotype. In some embodiments, the HBV is HBV-C1, HBV-QS-C2, HBV-C3, HBV-C4, HBV-C5, or HBV-C6-C15 genotype. In some embodiments, the HBV is HBV-D1, HBV-D2, HBV-D3, HBV-D4, HBV-D5, or HBV-D6 genotype. In some embodiments, the HBV is HBV-F1, HBV-F2, HBV-F3, or HBV-F4 genotype.

In some embodiments, the nucleic acid further comprises a promoter. In some embodiments, the promoter is a ubiquitous promoter. In some embodiments, the promoter is a tissue-specific promoter. In some embodiments, the promoter is a constitutive promoter. In some embodiments, the promoter is a human cytomegalovirus promoter.

In some embodiments, the nucleic acid further comprises an enhancer element. In some embodiments, the enhancer element is a human cytomegalovirus enhancer element.

In some embodiments, the nucleic acid further comprises a 5′ ITR element and 3′ ITR element.

In some embodiments, the vector is an adeno-associated virus (AAV) vector. In some embodiments, the adeno-associated virus (AAV) vector is AAV2, AAV5, AAV6, AAV7, AAV8, or AAV9. In some embodiments, the vector is an AAV6 vector or an AAV9 vector.

The vector can be any vector that is known in the art and is suitable for expressing the desired expression cassette. A number of vectors are known or can be designed to be capable of mediating transfer of gene products to mammalian cells, as is known in the art and described herein. In certain aspects, a vector refers to a nucleic acid polynucleotide to be delivered to a host cell, either in vitro or in vivo. In certain embodiments, the polynucleotide to be delivered comprises a coding sequence of interest in gene therapy (e.g. a Cas protein and gRNA). In some embodiments, a gene editing system are provided on a single vector. In some embodiments, a gene editing system are provided on a two or more vectors. In some embodiments, gene editing systems are provided by one or more vectors comprising an isolated nucleic acid encoding one or more elements of a gene editing system. In some embodiments, the CRISPR-Cas or SAM editing systems are provided by one or more vectors comprising an isolated nucleic acid encoding one or more elements of a CRISPR-Cas or SAM editing system. For example, in some embodiments, the composition comprises an isolated nucleic acid encoding a Cas protein and at least one guide nucleic acid (e.g., gRNA). In some embodiments, the composition comprises an isolated nucleic acid encoding a Cas9 protein, or functional fragment or derivative thereof. In some embodiments, the composition comprises at least one isolated nucleic acid encoding a Cas9 protein described elsewhere herein, or a functional fragment or derivative thereof. In some embodiments, the composition comprises at least one isolated nucleic acid encoding a Cas9 protein having at least 75%, 80%, 85%, 90%, 95%, 96%, 97%, 98%, or 99% amino acid sequence homology with a Cas9 protein described elsewhere herein. In some embodiments, the composition comprises an isolated nucleic acid encoding a CasX protein, or functional fragment or derivative thereof. In some embodiments, the composition comprises at least one isolated nucleic acid encoding a CasX protein described elsewhere herein, or a functional fragment or derivative thereof. In some embodiments, the composition comprises at least one isolated nucleic acid encoding a CasX protein having at least 75%, 80%, 85%, 90%, 95%, 96%, 97%, 98%, or 99% amino acid sequence homology with a CasX protein described elsewhere herein. In certain embodiments, the isolated nucleic acid comprises any type of nucleic acid, including, but not limited to DNA and RNA. For example, in some embodiments, the composition comprises an isolated DNA molecule, including for example, an isolated cDNA molecule, encoding a gRNA or protein of the described CRISPR-Cas systems or compositions, or functional fragment thereof. In some embodiments, the composition comprises an isolated RNA molecule encoding a protein of the described CRISPR-Cas systems or compositions, or a functional fragment thereof. In certain embodiments, the isolated nucleic acids are synthesized using any method known in the art.

In some instances, the expression of natural or synthetic nucleic acids encoding a RNA and/or peptide is typically achieved by operably linking a nucleic acid encoding the RNA and/or peptide or portions thereof to a promoter and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

In some embodiments, the vectors of the present disclosure are also used for nucleic acid immunization and gene therapy, using standard gene delivery protocols. Methods for gene delivery are known in the art. In another embodiment, the disclosure provides a gene therapy vector.

The isolated nucleic acid of the disclosed can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Additional promoter elements, e.g., enhancers, regulate the frequency of transcriptional initiation. In some embodiments, the vector also includes conventional control elements which are operably linked to the transgene in a manner which permits its transcription, translation and/or expression in a cell transfected with the plasmid vector or infected with the virus comprising a nucleic acid comprising the described CRISPR-Cas systems or compositions. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and can be utilized.

Typically, promoter elements (e.g., enhancers) are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

The selection of appropriate promoters can readily be accomplished. In certain aspects, one would use a high expression promoter. One example of a suitable promoter is the immediate early cytomegalovirus (CMV) promoter sequence. This promoter sequence is a strong constitutive promoter sequence capable of driving high levels of expression of any polynucleotide sequence operatively linked thereto. In certain embodiments, the Rous sarcoma virus (RSV) and MMT promoters are also be used. Certain proteins can be expressed using their native promoter. Other elements that can enhance expression can also be included such as an enhancer or a system that results in high levels of expression such as a tat gene and tar element. This cassette can then be inserted into a vector, e.g., a plasmid vector such as, pUC19, pUC118, pBR322, or other known plasmid vectors, that includes, for example, an E. coli origin of replication.

Another example of a suitable promoter is Elongation Growth Factor-1α (EF-1α). However, in some embodiments, other constitutive promoter sequences are used, including, but not limited to the simian virus 40 (SV40) early promoter, mouse mammary tumor virus (MMTV), human immunodeficiency virus (HIV) long terminal repeat (LTR) promoter, MoMuL V promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the disclosed should not be limited to the use of constitutive promoters. Inducible promoters are also contemplated as part of the disclosed. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. In some instances, enhancers are located upstream or downstream of the gene it regulates. In some instances, enhancers are also tissue-specific to enhance transcription in a specific cell or tissue type. In some embodiments, the vector of the present disclosure comprises one or more enhancers to boost transcription of the gene present within the vector. In some instances, the expression of the nucleic acid and/or protein, the expression vector to be introduced into a cell can also contain either a selectable marker gene or a reporter gene or both to facilitate identification and selection of expressing cells from the population of cells sought to be transfected or infected through viral vectors. In other embodiments, the selectable marker is carried on a separate piece of DNA and used in a co-transfection procedure. Both selectable markers and reporter genes can be flanked with appropriate regulatory sequences to enable expression in the host cells. Useful selectable markers include, for example, antibiotic-resistance genes, such as neo and the like.

Provided herein, in certain embodiments, are nucleic acids that encode any of the CRISPR-Cas systems described herein. For example, provided are vectors comprising a nucleic acid encoding: (a) a Clustered Regularly Interspaced Short Palindromic Repeat (CRISPR)-associated endonuclease; and (b) one or more guide RNAs (gRNAs) or a nucleic acid sequence encoding the one or more gRNAs, the one or more gRNA hybridizes or is complementary to a target nucleic acid sequence within a Hepatitis B Virus (HBV) genome, the HBV genome comprising at least about 90% sequence identity to any one of SEQ ID NOs: 2-5. In some embodiments, the target nucleic acid sequence comprises a sequence, the sequence comprising at least about 70%, 80%, 90%, 95%, 97%, 98%, or 99% sequence identity to any one of SEQ ID NOs: 2-5. In some embodiments, a gRNA of the one or more gRNAs is encoded by a sequence having at least 90%, 95%, 97%, 98%, or 99% sequence identity to any one of SEQ ID NOs: 6-549. In some embodiments, a gRNA of the one or more gRNAs is encoded by a sequence according to any one of SEQ ID NOs: 6-549. In some embodiments, a gRNA of the one or more gRNAs is encoded by a sequence according to any one of SEQ ID NOs: 6-549 comprising 1, 2, or 3 modifications. In some embodiments, a gRNA of the one or more gRNAs is encoded by a sequence having at least 90%, 95%, 97%, 98%, or 99% sequence identity to any one of SEQ ID NOs: 6-272. In some embodiments, a gRNA of the one or more gRNAs is encoded by a sequence according to any one of SEQ ID NOs: 6-272. In some embodiments, a gRNA of the one or more gRNAs is encoded by a sequence according to any one of SEQ ID NOs: 6-272 comprising 1, 2, or 3 modifications. In some embodiments, a gRNA of the one or more gRNAs is encoded by a sequence having at least 90%, 95%, 97%, 98%, or 99% sequence identity to any one of SEQ ID NOs: 273-548. In some embodiments, a gRNA of the one or more gRNAs is encoded by a sequence according to any one of SEQ ID NOs: 273-548. In some embodiments, a gRNA of the one or more gRNAs is encoded by a sequence according to any one of SEQ ID NOs: 273-548 comprising 1, 2, or 3 modifications. In some embodiments, the modification is a substitution, deletion, insertion, or a combination thereof.

In some embodiments, the CRISPR-associated endonuclease is Type I, Type II, or Type III Cas endonuclease. In other embodiments, the CRISPR-associated endonuclease is Cas3, Cas4, Cas5, Cas5e (or CasD), Cas6, Cas6e, CasΦf, Cas7, Cas8al, Cas8a2, Cas8b, Cas8c, Cas9, Cas10, Cas10d, CasF, CasG, CasH, CasX, CasΦ, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. By way of further example, in some embodiments, the CRISPR-Cas protein is a Cas1, Cas1B, Cas2, Cas3, Cas4, Cas5, Cash, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx16, CsaX, Csx3, Csx1, Csx15, Csf1, Csf2, Csf3, Csf4, Cas9, Cas12 (e.g., Cas12a, Cas12b, Cas12c, Cas12d, Cas12k, Cas12j/CasΦ, Cas12L etc.), Cas13 (e.g., Cas13a, Cas13b (such as Cas13b-t1, Cas13b-t2, Cas13b-t3), Cas13c, Cas13d, etc.), Cas14, CasX, CasY, or an engineered form of the Cas protein. In some embodiments, the CRISPR/Cas protein or endonuclease is Cas9. In some embodiments, the CRISPR/Cas protein or endonuclease is Cas12. In certain embodiments, the Cas12 polypeptide is Cas12a, Cas12b, Cas12c, Cas12d, Cas12e, Cas12g, Cas12h, Cas12i, Cas12L or Cas12J. In some embodiments, the CRISPR/Cas protein or endonuclease is CasX. In some embodiments, the CRISPR/Cas protein or endonuclease is CasY. In some embodiments, the CRISPR/Cas protein or endonuclease is CasΦ. In other embodiments, the CRISPR-associated endonuclease is a Cas9 endonuclease. In other embodiments, the Cas9 endonuclease is a Staphylococcus aureus Cas9 endonuclease. In other embodiments, the Cas9 endonuclease is a Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Fine goldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina Cas9 endonuclease. In some embodiments, the CRISPR-associated endonuclease is a CasX endonuclease. In some embodiments, the CasX endonuclease is a deltaproteobacteria CasX or a planctomycetes Cas X endonuclease. In some embodiments, the CasX endonuclease is a deltaproteobacteria CasX. In some embodiments, the CasX endonuclease is a planctomycetes Cas X endonuclease.

In some embodiments, the nucleic acid further comprises a promoter. In some embodiments, the promoter is a ubiquitous promoter. In some embodiments, the promoter is a tissue-specific promoter. In some embodiments, the promoter is a constitutive promoter. In some embodiments, the promoter is a human cytomegalovirus promoter. In some embodiments, the nucleic acid further comprises an enhancer element. In some embodiments, the enhancer element is a human cytomegalovirus enhancer element. In some embodiments, the nucleic acid further comprises a 5′ ITR element and 3′ ITR element.

In some embodiments, the adeno-associated virus (AAV) vector comprises an AAV2, AAV5, AAV6, AAV7, AAV8, or AAV9 capsid protein. In some embodiments, the adeno-associated virus (AAV) vector is AAV1, AAV2, AAV3, AAV4, AAV5, AAV6, AAV7, AAV8, AAV9, AAV10, AAV11, AAVDJ, or AAVDJ/8 capsid protein. In some embodiments, the nucleic acid comprising at least about 80, 85, 90, or 95% sequence identity to SEQ ID NO: 14. In some embodiments, the AAV vector is an AAV6 vector or an AAV9 vector.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art (see, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Regardless of the method used to introduce exogenous nucleic acids into a host cell, in order to confirm the presence of the recombinant nucleic acid sequence in the host cell, a variety of assays can be performed. Such assays include, for example, “molecular biological” assays well known to those of skill in the art, such as Southern and Northern blotting, RT-PCR and PCR; “biochemical” assays, such as detecting the presence or absence of a particular protein, e.g., by immunological means (ELISAs and Western blots) or by assays described herein to identify agents falling within the scope of the disclosure.

In some embodiments, a vector is provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, include but are not limited to, retroviruses, adenoviruses, adeno-associated viruses, herpes viruses, and lentiviruses. As described herein, a suitable vector generally contains an origin of replication functional in at least one organism, a promoter sequence, convenient restriction endonuclease sites, and one or more selectable markers.

Viral methods for introducing a polynucleotide of interest into a host cell include the use of DNA and RNA vectors. Viral vectors, and especially retroviral vectors, are useful for inserting genes into mammalian, e.g., human cells. Other viral vectors can be derived from lentivirus, poxviruses, herpes simplex virus I, adenoviruses and adeno-associated viruses, and the like. A number of viral based systems have been developed for gene transfer into mammalian cells. For example, retroviruses provide a convenient platform for gene delivery systems. A selected gene can be inserted into a vector and packaged in retroviral vectors using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. In another embodiment, non-AAV vectors are used, including integrating viruses, e.g., herpesvirus or lentivirus, although other viruses are selected. Suitably, where one of these other vectors is generated, it is produced as a replication-defective viral vector. In certain instances, replication-defective virus or viral vector refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In some embodiments, the genome of the viral vector does not include genes encoding the enzymes required to replicate (the genome can be engineered to be “gutless”—containing only the transgene of interest flanked by the signals required for amplification and packaging of the artificial genome), but these genes can be supplied during production.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Further provided are nucleic acids encoding the CRISPR-Cas systems described herein. Provided herein are adeno-associated virus (AAV) vectors comprising nucleic acids encoding the CRISPR-Cas systems described herein. In certain instances, an AAV vector includes to any vector that comprises or derives from components of AAV and is suitable to infect mammalian cells, including human cells, of any of a number of tissue types, such as brain, heart, lung, skeletal muscle, liver, kidney, spleen, or pancreas, whether in vitro or in vivo. In certain instances, an AAV vector includes an AAV type viral particle (or virion) comprising a nucleic acid encoding a protein of interest (e.g. CRISPR-Cas systems described herein). In some embodiments, as further described herein, the AAVs disclosed herein are be derived from various serotypes, including combinations of serotypes (e.g., “pseudotyped” AAV) or from various genomes (e.g., single-stranded or self-complementary). In some embodiments, the AAV vector is a human serotype AAV vector. In such embodiments, a human serotype AAV is derived from any known serotype, e.g., from AAV1, AAV2, AAV4, AAV6, or AAV9. In some embodiments, the serotype is AAV1, AAV2, AAV3, AAV4, AAV5, AAV6, AAV7, AAV8, AAV9, AAV10, AAV11, AAVDJ, or AAVDJ/8.

In some embodiments, the composition includes a vector derived from an adeno-associated virus (AAV). AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

A variety of different AAV capsids have been described and can be used, although AAV which preferentially target the liver and/or deliver genes with high efficiency are particularly desired. The sequences of the AAV8 are available from a variety of databases. While the examples utilize AAV vectors having the same capsid, the capsid of the gene editing vector and the AAV targeting vector are the same AAV capsid. Another suitable AAV is, e.g., rh10 (WO 2003/042397). Still other AAV sources include, e.g., AAV9 (see, for example, U.S. Pat. No. 7,906,111; US 2011-0236353-A1), and/or hu37 (see, e.g., U.S. Pat. No. 7,906,111; US 2011-0236353-A1), AAV1, AAV2, AAV3, AAV4, AAV5, AAV6, AAV6.2, AAV7, AAV8, (U.S. Pat. Nos. 7,790,449; 7,282,199, WO 2003/042397; WO 2005/033321, WO 2006/110689; U.S. Pat. Nos. 7,790,449; 7,282,199; 7,588,772). Still other AAV can be selected, optionally taking into consideration tissue preferences of the selected AAV capsid.

In some embodiments, AAV vectors disclosed herein include a nucleic acid encoding a CRISPR-Cas systems described herein. In some embodiments, the nucleic acid also includes one or more regulatory sequences allowing expression and, in some embodiments, secretion of the protein of interest, such as e.g., a promoter, enhancer, polyadenylation signal, an internal ribosome entry site (“IRES”), a sequence encoding a protein transduction domain (“PTD”), and the like. Thus, in some embodiments, the nucleic acid comprises a promoter region operably linked to the coding sequence to cause or improve expression of the protein of interest in infected cells. Such a promoter can be ubiquitous, cell- or tissue-specific, strong, weak, regulated, chimeric, etc., for example, to allow efficient and stable production of the protein in the infected tissue. In certain embodiments, the promoter is homologous to the encoded protein, or heterologous, although generally promoters of use in the disclosed methods are functional in human cells. Examples of regulated promoters include, without limitation, Tet on/off element-containing promoters, rapamycin-inducible promoters, tamoxifen-inducible promoters, and metallothionein promoters. In certain embodiments. other promoters used include promoters that are tissue specific for tissues such as kidney, spleen, and pancreas. Examples of ubiquitous promoters include viral promoters, particularly the CMV promoter, the RSV promoter, the SV40 promoter, etc., and cellular promoters such as the phosphoglycerate kinase (PGK) promoter and the b-actin promoter.

In some embodiments, the recombinant AAV vector comprises packaged within an AAV capsid, a nucleic acid, generally containing a 5′ AAV ITR, the expression cassettes described herein and a 3′ AAV ITR. As described herein, in some embodiments, an expression cassette contains regulatory elements for an open reading frame(s) within each expression cassette and the nucleic acid optionally contains additional regulatory elements. The AAV vector, in some embodiments, comprises a full-length AAV 5′ inverted terminal repeat (ITR) and a full-length 3′ ITR. A shortened version of 5′ ITR, termed ΔITR, has been described in which the D-sequence and terminal resolution site (trs) are deleted. The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription (see, for example, D M McCarty et al, “Self-complementary recombinant adeno-associated virus (scAAV) vectors promote efficient transduction independently of DNA synthesis”, Gene Therapy, (August 2001); see also, for example, U.S. Pat. Nos. 6,596,535; 7,125,717; and 7,456,683). Where a pseudotyped AAV is to be produced, the ITRs are selected from a source which differs from the AAV source of the capsid. For example, in some embodiments, AAV2 ITRs are selected for use with an AAV capsid having a particular efficiency for a selected cellular receptor, target tissue or viral target. In some embodiments, the ITR sequences from AAV2, or the deleted version thereof (ΔITR), are used for convenience and to accelerate regulatory approval (i.e. pseudotyped). In some embodiments, a single-stranded AAV viral vector is used.

Methods for generating and isolating AAV viral vectors suitable for delivery to a subject are known in the art (see, for example, U.S. Pat. Nos. 7,790,449; 7,282,199; WO 2003/042397; WO 2005/033321, WO 2006/110689; and U.S. Pat. No. 7,588,772 B2, U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065). In one system, a producer cell line is transiently transfected with a construct that encodes the transgene flanked by ITRs and a construct(s) that encodes rep and cap. In a second system, a packaging cell line that stably supplies rep and cap is transfected (transiently or stably) with a construct encoding the transgene flanked by ITRs. In each of these systems, AAV virions are produced in response to infection with helper adenovirus or herpesvirus, requiring the separation of the rAA Vs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors.

The CRISPR-Cas systems, for instance a Cas9 or CasX, and/or any of the present RNAs, for instance a guide RNA, can be delivered using adeno associated virus (AAV), lentivirus, adenovirus or other viral vector types, or combinations thereof. Cas9 or CasX and one or more guide RNAs can be packaged into one or more viral vectors. In some embodiments, the viral vector is delivered to the tissue of interest by, for example, an intramuscular injection, while other times the viral delivery is via intravenous, transdermal, intranasal, oral, mucosal, or other delivery methods. Such delivery can be either via a single dose, or multiple doses. One skilled in the art understands that the actual dosage to be delivered herein can vary greatly depending upon a variety of factors, such as the vector chose, the target cell, organism, or tissue, the general condition of the subject to be treated, the degree of transformation/modification sought, the administration route, the administration mode, the type of transformation/modification sought, etc.

Chemical means for introducing a polynucleotide into a host cell include colloidal dispersion systems, such as macromolecule complexes, nanocapsules, nanoparticles, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid can be associated with a lipid. The nucleic acid associated with a lipid is can be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they can be present in a bilayer structure, as micelles, or with a “collapsed” structure. They can also simply be interspersed in a solution, possibly forming aggregates that are not uniform in size or shape. Lipids are fatty substances which can be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

The compositions described herein are suitable for use in a variety of vector systems described above. Additionally, in order to enhance the in vivo serum half-life of the administered compound, the compositions can be encapsulated, introduced into the lumen of liposomes, prepared as a colloid, or other conventional techniques can be employed which provide an extended serum half-life of the compositions (see, for example, Szoka, et al., U.S. Pat. Nos. 4,235,871, 4,501,728 and 4,837,028). Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids can be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids can assume a micellar structure or merely exist as nonuniform aggregates of lipid molecules. Also contemplated are lipofectamine-nucleic acid complexes.

### Methods of Treatment

The gene editing systems provided herein are useful for methods of modifying and/or editing a HBV genome in the genome of a cell (e.g. host cell). In some embodiments, the gene editing systems (e.g., CRISPR-Cas) are designed to target a nucleic acid sequence in a HBV genome. In some embodiments, the target nucleic acid sequence is located within a structural gene, non-structural gene, or combinations thereof. In some embodiments, the target nucleic acid sequence is located within a C, X, P, or S region.

In some embodiments, the HBV is HBV-A genotype. In some embodiments, the HBV is HBV-B genotype. In some embodiments, HBV-C genotype. In some embodiments, the HBV is HBV-A, HBV-B, HBV-C, HBV-D, HBV-E, HBV-F, HBV-G, or HBV-H genotype. In some embodiments, the HBV is HBV-A1, HBV-A2, HBV-QS-A3, or HBV-A4 genotype. In some embodiments, the HBV is HBV-B1, HBV-B2, HBV-QS-B3, HBV-B4, or HBV-B5 genotype. In some embodiments, the HBV is HBV-C1, HBV-QS-C2, HBV-C3, HBV-C4, HBV-C5, or HBV-C6-C15 genotype. In some embodiments, the HBV is HBV-D1, HBV-D2, HBV-D3, HBV-D4, HBV-D5, or HBV-D6 genotype. In some embodiments, the HBV is HBV-F1, HBV-F2, HBV-F3, or HBV-F4 genotype.

Described herein, in some embodiments, are methods of modifying and/or editing a HBV genome in the genome of a cell (e.g. host cell) comprising administering compositions comprising (a) a Clustered Regularly Interspaced Short Palindromic Repeat (CRISPR)-associated endonuclease or a nucleic acid sequence encoding the CRISPR-associated endonuclease; and (b) one or more guide RNAs (gRNAs) or a nucleic acid sequence encoding the one or more gRNAs, the one or more gRNA hybridizes or is complementary to a target nucleic acid sequence within a Hepatitis B Virus (HBV) genome, the HBV genome comprising at least about 90% sequence identity to any one of SEQ ID NOs: 2-5. In some embodiments, the target nucleic acid sequence within a Hepatitis B Virus (HBV) genome, the HBV genome comprising at least about 70%, 75%, 80%, 85%, 90%, or 95% identity to any one of SEQ ID NOs: 2-5. In some embodiments, the HBV genome comprises a sequence of any one of SEQ ID NOs: 2-5. In some embodiments, a gRNA of the one or more gRNAs is encoded by a sequence having at least 90%, 95%, 97%, 98%, or 99% sequence identity to any one of SEQ ID NOs: 6-549. In some embodiments, a gRNA of the one or more gRNAs is encoded by a sequence according to any one of SEQ ID NOs: 6-549. In some embodiments, a gRNA of the one or more gRNAs is encoded by a sequence according to any one of SEQ ID NOs: 6-549 comprising 1, 2, or 3 modifications. In some embodiments, a gRNA of the one or more gRNAs is encoded by a sequence having at least 90%, 95%, 97%, 98%, or 99% sequence identity to any one of SEQ ID NOs: 6-272. In some embodiments, a gRNA of the one or more gRNAs is encoded by a sequence according to any one of SEQ ID NOs: 6-272. In some embodiments, a gRNA of the one or more gRNAs is encoded by a sequence according to any one of SEQ ID NOs: 6-272 comprising 1, 2, or 3 modifications. In some embodiments, a gRNA of the one or more gRNAs is encoded by a sequence having at least 90%, 95%, 97%, 98%, or 99% sequence identity to any one of SEQ ID NOs: 273-548. In some embodiments, a gRNA of the one or more gRNAs is encoded by a sequence according to any one of SEQ ID NOs: 273-548 In some embodiments, a gRNA of the one or more gRNAs is encoded by a sequence according to any one of SEQ ID NOs: 273-548sing 1, 2, or 3 modifications. In some embodiments, the modification is a substitution, deletion, insertion, or a combination thereof.

Provided herein, in certain embodiments, are methods of modifying and/or editing a HBV genome in the genome of a cell (e.g. host cell) using the CRISPR-Cas systems or compositions described herein. Generally, of modifying and/or editing a HBV DNA molecule (e.g. the HBV genome) in the genome of a cell (e.g. host cell) comprises contacting a cell, or providing to the cell, a CRISPR-Cas system or composition comprising one or more guide RNAs (gRNAs) or a nucleic acid sequence encoding the one or more gRNAs, the one or more gRNA hybridizes or is complementary to a target nucleic acid sequence within a Hepatitis B Virus (HBV) genome, the HBV genome comprising at least about 90% sequence identity to any one of SEQ ID NOs: 2-5. In certain instances, modulating or editing a HBV genome comprises removing and/or excising a polynucleotide sequence and/or region of the genome. In certain instances, modulating and editing comprises removing and/or excising a polynucleotide sequence and/or region sufficient to ablate or prevent the HBV genome from yielding a functional HBV gene product and/or a competent HBV virus (e.g. a functional HBV virus capable of replication in a host cell).

In some embodiments, the CRISPR-Cas system is encoded by a nucleic acid (e.g. a vector) and the cell is provided with the nucleic acid (e.g. via infection or transfection). In some embodiments, the nucleic acid is packaged in a viral vector.

In some embodiments, the cell is in a subject. In some specific embodiments, the subject is a human. In other specific embodiments, the subject is a non-human mammal. In other specific embodiments, the subject is any host that HBV may infect. In some embodiments, the HBV sequence is integrated into the cell. In some embodiments, the HBV sequence is in cytosol of the cell.

The methods disclosed herein further encompass, in some embodiments, administering a CRISPR-Cas system or composition. In certain instances, the pharmaceutical compositions comprising a CRISPR-Cas system or composition, as described herein, is administered. In certain instances, data obtained from the cell culture assays and animal studies can be used in formulating a range of dosage for use in humans. The dosage of such compositions lies preferably within a range of circulating concentrations that include the ED₅₀ with little or no toxicity. In some embodiments, the dosage varies within this range depending upon the dosage form employed and the route of administration utilized. For any composition used in the method of the described CRISPR-Cas systems or compositions, therapeutically effective dose can be estimated initially from cell culture assays. In certain embodiments, a dose is formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

The amount of a given agent that will correspond to such an amount will vary depending upon factors such as the particular compound, the severity of the disease, the identity (e.g., weight) of the subject or host in need of treatment, but can nevertheless be routinely determined in a manner known in the art according to the particular circumstances surrounding the case, including, e.g., the specific agent being administered, the route of administration, and the subject or host being treated. In certain instances, therapeutically effective amount and effective amount of a compound refer to an amount sufficient to provide a therapeutic benefit in the treatment, prevention and/or management of a disease, to delay or minimize one or more symptoms associated with the disease or disorder to be treated. In certain instances, therapeutically effective amount and effective amount encompass an amount that improves overall therapy, reduces or avoids symptoms or causes of disease or disorder, or enhances therapeutic efficacy of another therapeutic agent. In some embodiments, the desired dose(s) is (are) conveniently be presented in a single dose or as divided doses administered simultaneously (or over a short period of time) or at appropriate intervals, for example as two, three, four or more sub-doses per day.

In some embodiments, the cell is genetically modified in vivo in the subject in whom therapy is intended. In certain aspects, for in vivo, delivery the nucleic acid is injected directly into the subject. For example, In some embodiments, the nucleic acid is delivered at the site where the composition is required. In vivo nucleic acid transfer techniques include, but is not limited to, transfection with viral vectors such as adenovirus, Herpes simplex I virus, adeno-associated virus), lipid-based systems (useful lipids for lipid-mediated transfer of the gene are DOTMA, DOPE and DC-Chol, for example), naked DNA, and transposon-based expression systems. Exemplary gene therapy protocols see Anderson et al., Science 256:808-813 (1992). See also WO 93/25673 and the references cited therein. In some embodiments, the method comprises administering of RNA, for example mRNA, directly into the subject (see for example, Zangi et al., 2013 Nature Biotechnology, 31: 898-907).

For ex vivo treatment, an isolated cell is modified in an ex vivo or in vitro environment. In some embodiments, the cell is autologous to a subject to whom therapy is intended. Alternatively, the cell can be allogeneic, syngeneic, or xenogeneic with respect to the subject. The modified cells can then be administered to the subject directly.

One skilled in the art recognizes that different methods of delivery can be utilized to administer an isolated nucleic acid into a cell. Examples include: (1) methods utilizing physical means, such as electroporation (electricity), a gene gun (physical force) or applying large volumes of a liquid (pressure); and (2) methods wherein the nucleic acid or vector is complexed to another entity, such as a liposome, aggregated protein or transporter molecule.

The amount of vector to be added per cell will likely vary with the length and stability of therapeutic gene inserted in the vector, as well as also the nature of the sequence, and is particularly a parameter which needs to be determined empirically, and can be altered due to factors not inherent to the methods of the present disclosure (for instance, the cost associated with synthesis). One skilled in the art can easily make any necessary adjustments in accordance with the exigencies of the particular situation.

### Methods for Guide Identification

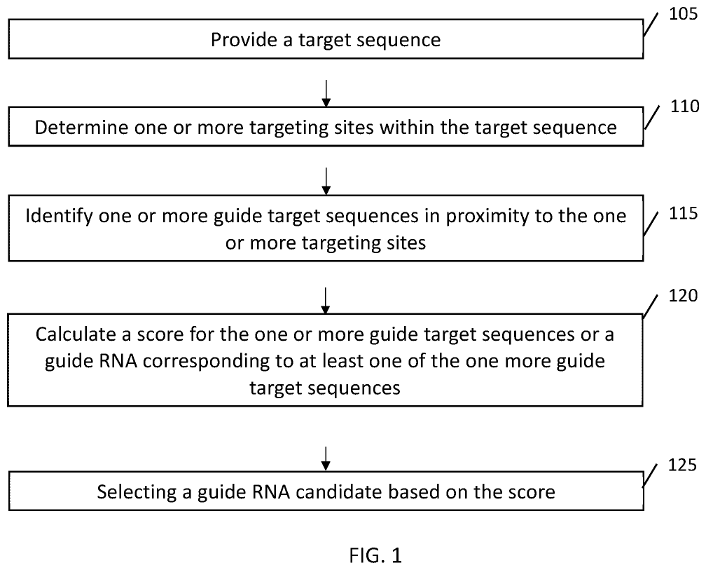

Referring to FIG. 1 is an exemplary flowchart of a method for selecting a guide RNA comprising a target sequence 105. In some embodiments, a target sequence comprises a nucleotide sequence for selecting a guide RNA. In some cases, the target sequence comprises at least 5, 10, 15, 20, 25, 30, 35, or 40 nucleotides. In some cases, the target sequence comprises about 5, 10, 15, 20, 25, 30, 35, or 40 nucleotides. In some embodiments, the target sequences comprise a reference sequence. In some embodiments, the target sequence comprises a single natural or computed sequence. In some embodiments, the target sequence is a single natural sequence. In some embodiments, the target sequence is a single computed sequence. In some embodiments, the target sequence is generated by aligning a plurality of sequences. In some cases, the plurality of sequences comprises at least 5, 20, 50, 80, 100, 200, 400, 500, 800, or 1000 sequences. In some cases, the plurality of sequences comprises about 5, 20, 50, 80, 100, 200, 400, 500, 800, or 1000 sequences. In some embodiments, the consensus sequence generated from the plurality of sequences is a non-naturally occurring sequence. In some embodiments, the consensus sequence comprises ambiguity (e.g., different nucleotides at each position). In some embodiments, the consensus sequence comprises at least or about 5%, 10%, 15%, 20%, 25%, 30%, 35%, 40%, 45%, 50%, 55%, 60%, or more than 60% ambiguity. In some embodiments, the consensus sequence comprises less than about 5%, 10%, 15%, 20%, 25%, 30%, or more than 30% difference as compared to the naturally occurring sequence.

In some embodiments, the target sequence (e.g., a reference sequence or a plurality of sequences) is found in a data storage system (e.g., database), such as those described herein. In some embodiments, the data storage system comprises a centralized system. In some embodiments, the data storage system comprises a decentralized system. In some embodiments, the data storage system comprises a cloud-based storage system (e.g., iCloud, AWS, Dropbox, Google Drive, OneDrive, etc.). In some cases, the cloud-based storage system is a private cloud storage, a public cloud storage, a hybrid cloud storage, or a community cloud storage system.

In some cases, the target sequence is generated by aligning a plurality of sequences. In some cases, the plurality of sequence is aligned through a computer program (e.g., Clustal Omega). In some instances, the target sequence is a consensus sequence determined by aligning the plurality of sequences. In some examples, the consensus sequence is determined by aligning sequences with common geographies, pathologies, other attributes, or combinations thereof. In some cases, the consensus is determined by comparing nucleotides at each position of the plurality of sequences and selecting a nucleotide at each position that appears at a frequency higher than other nucleotides. In some embodiments, the target sequence is a consensus sequence generated by a module configured to align the plurality of sequences. In some cases, the module is further configured to compare nucleotides at each position of the plurality of sequences; and select a nucleotide at each position that appears at a frequency higher than other nucleotides. In some examples, any one of A, T, G, and C appear at a higher frequency than the other three nucleotides at a given position in the plurality of sequences.

In some embodiments, the plurality of sequences aligned to generate the target sequence is a plurality of viral sequences. In some cases, the plurality of viral sequences are sequences from a single virus. In some cases, the plurality of viral sequences are sequences from different viruses. In some instances, the plurality of viral sequences are sequences within a same genotype of a virus. In some instances, the plurality of viral sequences are sequences within different genotypes of a virus. In some examples, the plurality of viral sequences are sequences within different subclades of a virus. In some examples, the plurality of viral sequences are sequences within a same subclade of a virus.

In some embodiments, a virus comprises Hepatitis B virus (HBV), Human Immunodeficiency virus (HIV), JC virus (JCV), herpes simplex virus (HSV), or SARS-CoV-2. In some cases, the virus is Hepatitis B virus (HBV) and the genotype is HBV-A. In some cases, the virus is Hepatitis B virus (HBV) and the genotype is HBV-B. In some cases, the virus is Hepatitis B virus (HBV) and the genotype is HBV-C. In some cases, the virus is Hepatitis B virus (HBV) and the different genotypes comprise HBV-A, HBV-B, HBV-C, or combinations thereof. In some cases, the virus is Hepatitis B virus (HBV) and different genotypes comprise HBV-A, HBV-B, HBV-C, HBV-D, HBV-E, HBV-F, HBV-G, HBV-H, or combinations thereof. In some cases, the virus is Hepatitis B virus (HBV) and various subclades within HBV-A, HBV-B, HBV-C, HBV-D, HBV-E, HBV-F, HBV-G, HBV-H, or combinations thereof.

In some instances, the subclade within HBV-A comprises HBV-A1, HBV-A2, HBV-QS-A3, HBV-A4, or combinations thereof. In some instances, the subclade within HBV-B comprises HBV-B1, HBV-B2, HBV-QS-B3, HBV-B4, HBV-B5, or combinations thereof. In some instances, the subclade within HBV-C comprises HBV-C1, HBV-QS-C2, HBV-C3, HBV-C4, HBV-C5, HBV-C6-C15, or combinations thereof. In some instances, the subclade within HBV-D comprises HBV-D1, HBV-D2, HBV-D3, HBV-D4, HBV-D5, HBV-D6, or combinations thereof. In some instances, the subclade within HBV-F comprises HBV-F1, HBV-F2, HBV-F3, HBV-F4, or combinations thereof.

In some embodiments, HBV genotypes and subgenotypes/subclades in populations differ between geographic regions. As an example, the subclades in North America include HBV-A2, HBV-D2, HBV-B5, HBV-B4, and HBV-G. As an example, the subclades in Central America include HBV-A2, HBV-F1, HBV-H, HBV-G, HBV-B2, HBV-F3, HBV-C1, and HBV-F4. As an example, the subclades in Caribbean include HBV-A1, HBV-QS-A3, HBV-D4, HBV-A2, and HBV-D3. As an example, the subclades in South America include HBV-F1, HBV-F4, HBV-D3, HBV-F3, HBV-F2, HBV-A1, HBV-A2, and HBV-D2. As an example, the subclades in Northern Europe include HBV-D2, HBV-A2, HBV-D3, and HBV-E. As an example, the subclades in Southern Europe include HBV-D3, HBV-D2, HBV-D1, and HBV-A2. As an example, the subclades in Western Europe include HBV-A2, HBV-D1, HBV-D2, HBV-D3, and HBV-E. As an example, the subclades in Eastern Europe include HBV-D2, HBV-A2, HBV-D1, and HBV-D3. As an example, the subclades in Northern Africa include HBV-D1, HBV-E, HBV-D6, HBV-D2, and HBV-D3. As an example, the subclades in Western Africa include HBV-E and HBV-A2. As an example, the subclades in Middle Africa include HBV-E, and HBV-QS-A3. As an example, the subclades in Eastern Africa include HBV-A1, HBV-D2, and HBV-E. As an example, the subclades in Southern Africa include HBV-A1, HBV-D3, HBV-E, and HBV-A2. As an example, the subclades in Western Asia include HBV-D1 and HBV-D2. As an example, the subclades in Southern Asia include HBV-D1, HBV-D3, HBV-D2, HBV-A1, HBV-C1, and HBV-D5. As an example, the subclades in Central Asia include HBV-D1, HBV-D2, HBV-QS-C2, and HBV-A2. As an example, the subclades in Eastern Asia include HBV-QS-C2, HBV-B2, HBV-C1, HBV-QS-B3, and HBV-C6-C15. As an example, the subclades in Southeastern Asia include HBV-C1, HBV-B2, HBV-QS-B3, HBV-B4, and HBV-QS-C2. As an example, the subclades in Melanesia include HBV-D2, HBV-C3, and HBV-C6-C15. As an example, the subclades in Polynesia include HBV-C3. As an example, the subclades in Australia and New Zealand include HBV-D1, HBV-C4, HBV-C3, and HBV-D4. In some embodiments, the most frequently observed subclades for various geographic regions, in decreasing order, comprise, for North America: HBV-A2>HBV-D2>HBV-B5>HBV-B4>HBV-G; for Central America: HBV-A2>HBV-F1>HBV-H>HBV-G>HBV-B2>HBV-F3>HBV-C1>HBV-F4; for Caribbean: HBV-A1>HBV-QS-A3>HBV-D4>HBV-A2>D3; for South America: HBV-F1>HBV-F4>HBV-D3>HBV-F3>HBV-F2>HBV-A1>HBV-A2>HBV-D2; for Northern Europe: HBV-D2>HBV-A2>HBV-D3>HBV-E; for Southern Europe: HBV-D3>HBV-D2>HBV-D1>HBV-A2; for Western Europe: HBV-A2>HBV-D1>HBV-D2>HBV-D3>HBV-E; for Eastern Europe: HBV-D2>HBV-A2>HBV-D1>HBV-D3; for Northern Africa: HBV-D1>HBV-E>HBV-D6>HBV-D2>HBV-D3; for Western Africa: HBV-E>HBV-A2; for Middle Africa: HBV-E>HBV-QS-A3; for Eastern Africa: HBV-A1>HBV-D2>HBV-E; for Southern Africa: HBV-A1>HBV-D3>HBV-E>HBV-A2, for Western Asia: HBV-D1>HBV-D2, for Southern Asia: HBV-D1>HBV-D3>HBV-D2>HBV-A1>HBV-C1>HBV-D5; for Central Asia: HBV-D1>HBV-D2>HBV-QS-C2>HBV-A2; for Eastern Asia: HBV-QS-C2>HBV-B2>HBV-C1>HBV-QS-B3>HBV-C6-C15; Southeastern Asia: HBV-C1>HBV-B2>HBV-QS-B3>B4>HBV-QS-C2; Melanesia: HBV-D2>HBV-C3>HBV-C6-C15; Polynesia: HBV-C3; and for Australia and New Zealand: HBV-D1>HBV-C4>HBV-C3>HBV-D4.

### Identifying a Targeting Site

In some embodiments, a target sequence (e.g., consensus sequence) is provided and one or more targeting sites that can be recognized by a zinc finger nuclease, a transcription activator-like effector nuclease (TALEN), a meganuclease, or a CRISPR-associated protein (e.g., targeting site adjacent or proximal to PAMs) are determined within the target sequence 110. In some embodiments, the presence of a targeting site (e.g., targeting site adjacent or proximal to PAMs) is indicative of a cut prediction by a nuclease (e.g., zinc finger nuclease, a transcription activator-like effector nuclease (TALEN), a meganuclease, or a CRISPR-associated protein) within the target sequence. In some cases, the targeting site (e.g., targeting site adjacent or proximal to PAMs) is approximately 5, 10, 15, 20, 25, or 30 nucleotides downstream of a sequence targeted by a guide RNA. In some cases, the targeting site is approximately 2, 3, 4, 5, or 6 nucleotides downstream of a sequence targeted by a guide RNA (e.g., PAM). In some cases, the targeting site is approximately 5, 10, 15, 20, 25, or 30 nucleotides upstream of a sequence targeted by a guide RNA (e.g., PAM). In some cases, the targeting site is approximately 2, 3, 4, 5, or 6 nucleotides upstream of where the target sequence is cut by a nuclease. In some instances, the nuclease comprises a zinc finger nuclease, a transcription activator-like effector nuclease (TALEN), a meganuclease, or a CRISPR-associated protein (e.g., Cas). In some instances, the nuclease comprises a CRISPR-associated protein (e.g., Cas). In some embodiments, the one or more target sites are determined with one or more modules (e.g., a target site identifier). In some embodiments, the one or more PAMs are determined with one or more modules (e.g., a PAM identifier). In some cases, the one or more modules are communicably coupled to a data storage system (e.g., a database) comprising a target sequence, such as those described herein.

In some embodiments, the targeting site is a site in the target sequence to which a programmable DNA-binding domain (pDBD) binds. In some embodiments, the one or more targeting sites comprise one or more pDBD binding sites in the target sequence. In some embodiments, such one or more pDBD binding sites are located at different positions and orientations relative to the nuclease target site (e.g., targeting site adjacent or proximal to PAMs). In some embodiments, the one or more targeting sites comprise one or more pDBD binding sites and one or more PAMs. As an example, for a given genotype, a corresponding target sequence (e.g., a consensus sequence) is scanned through to compile all candidate guide RNAs that include a PAM sequence (e.g., SaCas9 PAM sequence). In some embodiments, to compile guide RNAs targeting the (+) strand of the cccDNA, 30-basepair (bp) DNA sequences composed of a 24-bp protospacer and 5′-NNGRRT-3′ PAM sequences are selected. In some further embodiments, to compile guide RNAs targeting the (−) strand of the cccDNA, 30-bp DNA sequences composed of 5′-AYYCNN-3′ PAM sequence and a 24-bp protospacer are selected. In some instances, candidate guide RNAs with ‘-’ in protospacer is eliminated for downstream analysis afterwards.

In some instances, during a PAM finding step, the protospacers and PAM sequence are selected based on a downstream analysis. As an example, a 24-bp protospacer with 6-bp PAM sequence is selected if the downstream analysis comprises an on-target knockout scoring with Azimuth 2.0 algorithm, which requires a 30-bp input sequence. In some examples, downstream in silico evaluation processes are measuring only the 20-bp protospacer of interest with a 6-bp PAM sequence.

In some embodiments, one or more guide target sequences are identified that are in proximity to the one or more target sites (e.g., targeting site adjacent or proximal to PAMs) 115. In some cases, the one or more modules are further configured to identify the one or more guide target sequences in proximity to the one or more target sites (e.g., targeting site adjacent or proximal to PAMs). In some instances, the one or more modules comprises a proximity identifier. In some cases, the one or more guide target sequences comprises a guide RNA candidate.

As used herein, “proximity” or “proximal” refers to a distance within 5, 10, 15, 20, 25, 30, 35, or 40 nucleotides or base pairs (bps). In some embodiments, “proximity” or “proximal” refers to a distance of 5, 10, 15, 20, 25, 30, 35, or 40 nucleotides or bps upstream or downstream of a position or sequence (e.g., a targeting site). In some embodiments, “proximity” or “proximal” refers to a distance of 5, 10, 15, 20, 25, 30, 35, or 40 nucleotides or bps upstream or downstream of a targeting site. In some embodiments, “proximity” or “proximal” refers to a distance of 2, 3, 4, 5, 6, 7, 8, 9 or 10 nucleotides or bps upstream or downstream of a position or sequence (e.g., a targeting site). In some embodiments, “proximity” or “proximal” refers to a distance of 2, 3, 4, 5, or 6 nucleotides or bps upstream or downstream of a position or sequence (e.g., a targeting site). In some embodiments, “proximity” or “proximal” refers to a distance of 3 or 4 nucleotides or bps upstream or downstream of a position or sequence (e.g., a targeting site).

In some embodiments, in silico evaluation of a guide RNA's likelihood of an on-target cleavage is assessed using a position-specific penalty matrix comprising mismatches across the protospacer. In some cases, protospacers proximal to the target site sequence is more deleterious than those distal to the target site (e.g., targeting site adjacent or proximal to PAMs). In some instances, penalty scores are assigned to mismatches within the PAM sequence (e.g., SaCas9 PAM sequence). An exemplary algorithm to compute a set of mismatch scores for each guide RNA comprises:
a. for each viral sequence of a given genotype (e.g., HBV genotype), find the best match of this guide RNA (e.g., shortest Levenshtein distance);
b. apply an additive penalty function across all guide RNA-target pairs in the ungapped sequence to compute individual guide RNA mismatch scores (e.g., score 0 indicates a perfect match);
c. summarize the distribution of mismatch scores for each guide RNA into bins (e.g., five bins, six bins, seven bins, etc.);
d. use the mismatch scores to eliminate candidate guide RNAs with low probabilities of target site cleavage.

In some cases, the pseudocode for in silico evaluation of a guide RNAs' likelihood of on-target cleavage based on the algorithm above comprises:

| | |
|---|---|
| 1 | position-specific penalty vector = [penalty₁, penalty₂, ..., penalty₂₆] |
| 2 | for each guide RNA i: |
| 3 | for each viral DNA sequence j of a given genotype in a data storage system: |
| 4 | for each position k of the (RNA, DNA) pair: |
| 5 | if RNAₖ matches DNAₖ: penalty₁ = 0 |
| 6 | else: penaltyₖ = penalty vector [k] |
| 7 | calculate mismatch scoreᵢⱼ = penalty₁ + penalty₂ + ... + penalty₂₆ |
| 8 | mismatch score vector for RNA i = [mms_{i,1}, mms_{i,2, ...} , mms_{i,3000+} ] |
| 9 | plot distribution of all mismatch scores for RNA i |
| 10 | summarize the distribution of mismatch scores for RNA i with 6 probabilities for |
| | 6 bins: |
| 11 | probabilities = [P_{mms = 0}, P_{0 < mms <= 1}, P_{0 < mms <= 2}, P_{0 < mms <= 3}, P_{0 < mms <= 5}, P_{0 < mms <= 10}, P_{mms > 10}] |
| 12 | eliminate guide RNAs with low probabilities of target site cleavage |

In some embodiments, the one or more guide RNA (gRNA) candidates generated from the target sequence are about 15 nucleotides to about 28 nucleotides. In some embodiments, the gRNA comprises at least about 15 nucleotides. In some embodiments, the gRNA comprises at most about 28 nucleotides. In some embodiments, the gRNA comprises about 15 nucleotides to about 16 nucleotides, about 15 nucleotides to about 17 nucleotides, about 15 nucleotides to about 18 nucleotides, about 15 nucleotides to about 19 nucleotides, about 15 nucleotides to about 20 nucleotides, about 15 nucleotides to about 21 nucleotides, about 15 nucleotides to about 22 nucleotides, about 15 nucleotides to about 23 nucleotides, about 15 nucleotides to about 24 nucleotides, about 15 nucleotides to about 25 nucleotides, about 15 nucleotides to about 28 nucleotides, about 16 nucleotides to about 17 nucleotides, about 16 nucleotides to about 18 nucleotides, about 16 nucleotides to about 19 nucleotides, about 16 nucleotides to about 20 nucleotides, about 16 nucleotides to about 21 nucleotides, about 16 nucleotides to about 22 nucleotides, about 16 nucleotides to about 23 nucleotides, about 16 nucleotides to about 24 nucleotides, about 16 nucleotides to about 25 nucleotides, about 16 nucleotides to about 28 nucleotides, about 17 nucleotides to about 18 nucleotides, about 17 nucleotides to about 19 nucleotides, about 17 nucleotides to about 20 nucleotides, about 17 nucleotides to about 21 nucleotides, about 17 nucleotides to about 22 nucleotides, about 17 nucleotides to about 23 nucleotides, about 17 nucleotides to about 24 nucleotides, about 17 nucleotides to about 25 nucleotides, about 17 nucleotides to about 28 nucleotides, about 18 nucleotides to about 19 nucleotides, about 18 nucleotides to about 20 nucleotides, about 18 nucleotides to about 21 nucleotides, about 18 nucleotides to about 22 nucleotides, about 18 nucleotides to about 23 nucleotides, about 18 nucleotides to about 24 nucleotides, about 18 nucleotides to about 25 nucleotides, about 18 nucleotides to about 28 nucleotides, about 19 nucleotides to about 20 nucleotides, about 19 nucleotides to about 21 nucleotides, about 19 nucleotides to about 22 nucleotides, about 19 nucleotides to about 23 nucleotides, about 19 nucleotides to about 24 nucleotides, about 19 nucleotides to about 25 nucleotides, about 19 nucleotides to about 28 nucleotides, about 20 nucleotides to about 21 nucleotides, about 20 nucleotides to about 22 nucleotides, about 20 nucleotides to about 23 nucleotides, about 20 nucleotides to about 24 nucleotides, about 20 nucleotides to about 25 nucleotides, about 20 nucleotides to about 28 nucleotides, about 21 nucleotides to about 22 nucleotides, about 21 nucleotides to about 23 nucleotides, about 21 nucleotides to about 24 nucleotides, about 21 nucleotides to about 25 nucleotides, about 21 nucleotides to about 28 nucleotides, about 22 nucleotides to about 23 nucleotides, about 22 nucleotides to about 24 nucleotides, about 22 nucleotides to about 25 nucleotides, about 22 nucleotides to about 28 nucleotides, about 23 nucleotides to about 24 nucleotides, about 23 nucleotides to about 25 nucleotides, about 23 nucleotides to about 28 nucleotides, about 24 nucleotides to about 25 nucleotides, about 24 nucleotides to about 28 nucleotides, or about 25 nucleotides to about 28 nucleotides. In some embodiments, the gRNA comprises about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, or about 28 nucleotides.

In some embodiments, the guide RNA candidate is compared against the consensus sequence or the plurality of sequences. In some embodiments, the guide RNA candidate is compared against a sequence of a viral strain different from the plurality of sequences. In further embodiments, the guide RNA candidate is compared against a sequence of a human genome different from the plurality of sequence.

In some cases, the guide RNA candidate hybridizes to a section of the target sequence (e.g., consensus sequence). In some cases, the guide RNA candidate is complementary sequence to a section of the target sequence. In some embodiments, the target sequence has about 2000 nucleotides to about 4200 nucleotides. In some embodiments, the gRNA comprises at least about 2000 nucleotides. In some embodiments, the gRNA comprises at most about 4000 nucleotides. In some embodiments, the gRNA comprises about 2000 nucleotides to about 2200 nucleotides, about 2000 nucleotides to about 2400 nucleotides, about 2000 nucleotides to about 2600 nucleotides, about 2000 nucleotides to about 2800 nucleotides, about 2000 nucleotides to about 3000 nucleotides, about 2000 nucleotides to about 3200 nucleotides, about 2000 nucleotides to about 3400 nucleotides, about 2000 nucleotides to about 3600 nucleotides, about 2000 nucleotides to about 3800 nucleotides, about 2000 nucleotides to about 4000 nucleotides, about 2000 nucleotides to about 4200 nucleotides, about 2200 nucleotides to about 2400 nucleotides, about 2200 nucleotides to about 2600 nucleotides, about 2200 nucleotides to about 2800 nucleotides, about 2200 nucleotides to about 3000 nucleotides, about 2200 nucleotides to about 3200 nucleotides, about 2200 nucleotides to about 3400 nucleotides, about 2200 nucleotides to about 3600 nucleotides, about 2200 nucleotides to about 3800 nucleotides, about 2200 nucleotides to about 4000 nucleotides, about 2200 nucleotides to about 4200 nucleotides, about 2400 nucleotides to about 2600 nucleotides, about 2400 nucleotides to about 2800 nucleotides, about 2400 nucleotides to about 3000 nucleotides, about 2400 nucleotides to about 3200 nucleotides, about 2400 nucleotides to about 3400 nucleotides, about 2400 nucleotides to about 3600 nucleotides, about 2400 nucleotides to about 3800 nucleotides, about 2400 nucleotides to about 4000 nucleotides, about 2400 nucleotides to about 4200 nucleotides, about 2600 nucleotides to about 2800 nucleotides, about 2600 nucleotides to about 3000 nucleotides, about 2600 nucleotides to about 3200 nucleotides, about 2600 nucleotides to about 3400 nucleotides, about 2600 nucleotides to about 3600 nucleotides, about 2600 nucleotides to about 3800 nucleotides, about 2600 nucleotides to about 4000 nucleotides, about 2600 nucleotides to about 4200 nucleotides, about 2800 nucleotides to about 3000 nucleotides, about 2800 nucleotides to about 3200 nucleotides, about 2800 nucleotides to about 3400 nucleotides, about 2800 nucleotides to about 3600 nucleotides, about 2800 nucleotides to about 3800 nucleotides, about 2800 nucleotides to about 4000 nucleotides, about 2800 nucleotides to about 4200 nucleotides, about 3000 nucleotides to about 3200 nucleotides, about 3000 nucleotides to about 3400 nucleotides, about 3000 nucleotides to about 3600 nucleotides, about 3000 nucleotides to about 3800 nucleotides, about 3000 nucleotides to about 4000 nucleotides, about 3000 nucleotides to about 4000 nucleotides, about 3200 nucleotides to about 3400 nucleotides, about 3200 nucleotides to about 3600 nucleotides, about 3200 nucleotides to about 3800 nucleotides, about 3200 nucleotides to about 4000 nucleotides, about 3200 nucleotides to about 4200 nucleotides, about 3400 nucleotides to about 3600 nucleotides, about 3400 nucleotides to about 3800 nucleotides, about 3400 nucleotides to about 4000 nucleotides, about 3400 nucleotides to about 4200 nucleotides, about 3600 nucleotides to about 3800 nucleotides, about 3600 nucleotides to about 4000 nucleotides, about 3600 nucleotides to about 4200 nucleotides, about 3800 nucleotides to about 4000 nucleotides, about 3800 nucleotides to about 4200 nucleotides, or about 4000 nucleotides to about 4200 nucleotides. In some embodiments, the gRNA comprises about 2000 nucleotides, about 2200 nucleotides, about 2400 nucleotides, about 2600 nucleotides, about 2800 nucleotides, about 3000 nucleotides, about 3200 nucleotides, about 3400 nucleotides, about 3600 nucleotides, about 3800 nucleotides, about 4000 nucleotides, or about 4200 nucleotides.

In some embodiments, the target sequence (e.g., consensus sequence) comprises at least about 70%, 75%, 80%, 85%, 90% or 95% sequence identity to any one of SEQ ID NOs: 2-5 of Table 1. In some embodiments, the guide RNA candidate is encoded by a sequence according to any one of SEQ ID NOs: 6-290 of Table 1. In some embodiments, the guide RNA candidate is encoded by a sequence according to any one of SEQ ID NOs: 6-14 of Table 1. In some embodiments, the guide RNA candidate is encoded by a sequence according to any one of SEQ ID NOs: 15-290 of Table 1.

### Criteria for Selecting a Guide RNA

In some embodiments, one or more criteria are calculated based on the one or more guide target sequences or a guide RNA corresponding to at least one of the one more guide target sequence. In some cases, the one or more criteria are used to calculate a score for the one or more guide target sequences or a guide RNA corresponding to at least one of the one more guide target sequences 120, as exemplary illustrated in FIG. 1. In some embodiments, the one or more criteria are used to select a guide sequence of interest from the one or more guide target sequences. In some embodiments, the one or more criteria are used to select a guide RNA from the candidate guide RNAs. In some embodiments, the guide RNA candidate is selected based on a score 125.

In some embodiments, the one or more criteria are user-supplied criteria. In some embodiments, the one or more criteria are based on positional entropy, conservation, a knockout score, an overlapping reading frame, a gene location, a coding region, a non-coding region, predicted cutting rate or efficiency, efficacy, a frequency of a PAM site, or combinations thereof. In some cases, the positional entropy comprises Shannon entropy. In some instances, a positional frequency matrix is generated from sequence alignments described herein. In some cases, the positional frequency matrix illustrates the contribution of different nucleotides or combinations (e.g., “A”, “T”, “C”, “G”, “N”, “−”, etc.) in each position of the sequence alignments. For each position, positional Shannon entropy is calculated based on the positional frequency matrix, for example, according to the equation:

$$Entropy = - {\sum }_{i = A , T , G , C} ( {p}_{i} * {log}_{2} ( {p}_{i} ) )$$

$${p}_{i} = \frac{{f}_{i} + 1}{( {\sum }_{i = A , T , G , C} {f}_{i} ) + 1}$$

where fᵢ is the frequency of a given nucleotide i for a given position. In some embodiments, an average Shannon entropy is computed as a moving average of at least a 10, 15, 20, 25, or 30-nt window. In some embodiments, an average Shannon entropy is computed as a moving average of about a 10, 15, 20, 25, or 30-nt window. In some embodiments, an average Shannon entropy is computed as a moving average of a 26-nt window.

In some cases, the knockout score is an on-target knockout score. In some cases, the on-target knockout score is determined by efficiency of cleavage. In some cases, the efficiency of cleavage is based at least in part the one or more PAMs. In some instances, the on-target knockout score is determined using an algorithm (e.g., Azimuth 2.0). In some cases, the knockout score is an off-target knockout score. In some instances, the off-target knockout score is determined using an algorithm (e.g., COSMID, python script, Cas-OFFinder). In some embodiments, the one or more modules comprise a criteria analysis module configured to calculate the one or more criteria described herein. In some cases, the criteria analysis module comprises an algorithm described herein (e.g., Azimuth 2.0, COSMID, python script, Cas-OFFinder, etc.).

In some embodiments, the one or more criteria, such as those described herein, are used to select a guide RNA. In some cases, the guide RNA is referred to as a candidate guide RNA. In some instances, the guide RNA is selected based on a score, such as those described herein. In some cases, the guide RNA candidate is selected by a user. In some cases, the guide RNA candidate is selected by a module configured to analyze the one or more criteria and select the guide RNA candidate based on the one or more criteria. In some cases, the guide RNA candidate is a clinical candidate.

### Computer Systems

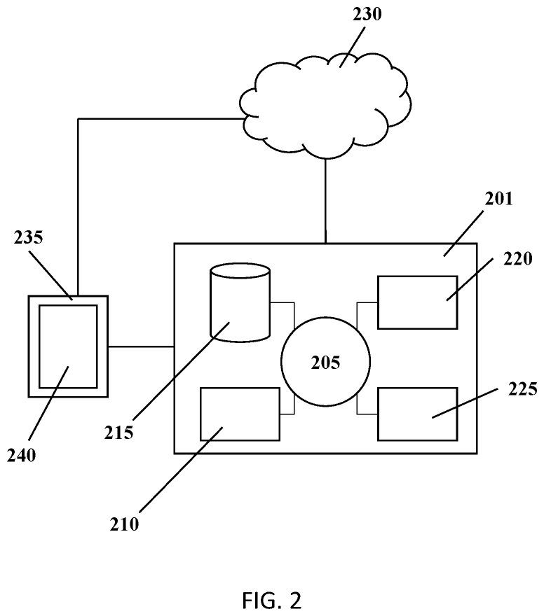

In an aspect, the present disclosure provides computer systems that are programmed or otherwise configured to implement methods of the disclosure, e.g., any of the subject methods for medical imaging. FIG. 2 shows a computer system 201 that is programmed or otherwise configured to implement a method for selecting a guide RNA. In some embodiments, the computer system 201 is configured to, for example, (i) identify one or more target sites (e.g., targeting site adjacent or proximal to PAMs) in the target sequence (e.g., consensus sequence), (ii) identify one or more guide target sequences in proximity to the one or more target sites (e.g., targeting site adjacent or proximal to PAMs), and (iii) calculate one or more criteria based on the one or more guide target sequences or a guide RNA corresponding to at least one of the one more guide target sequence. In some embodiments, the computer system 201 is an electronic device of a user or a computer system that is remotely located with respect to the electronic device. In some embodiments, the electronic device is a mobile electronic device.

In some embodiments, the computer system 201 includes a central processing unit (CPU, also “processor” and “computer processor” herein) 205. In some embodiments, the CPU is a single core or multi core processor, or a plurality of processors for parallel processing. In some embodiments, the computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. In some embodiments, the storage unit 215 is a data storage unit (or data repository) for storing data. In some embodiments, the computer system 201 is operatively coupled to a computer network (“network”) 230 with the aid of the communication interface 220. In some embodiments, the network 230 is the Internet, an internet and/or extranet, or an intranet and/or extranet that is in communication with the Internet. In some embodiments, the network 230 in some cases is a telecommunication and/or data network. In some embodiments, the network 230 includes one or more computer servers, which enable distributed computing, such as cloud computing. In some embodiments, the network 230, in some cases with the aid of the computer system 201, implements a peer-to-peer network, which enables devices coupled to the computer system 201 to behave as a client or a server.

In some embodiments, the CPU 205 executes a sequence of machine-readable instructions. In some embodiments, the sequence of machine-readable instructions are embodied in a program or software. In some embodiments, the instructions are stored in a memory location, such as the memory 210. In some embodiments, the instructions are directed to the CPU 205, which subsequently program or otherwise configure the CPU 205 to implement methods of the present disclosure. Examples of operations performed by the CPU 205 include fetch, decode, execute, and writeback.

In some embodiments, the CPU 205 is part of a circuit, such as an integrated circuit. In some embodiments, one or more other components of the system 201 are included in the circuit. In some cases, the circuit is an application specific integrated circuit (ASIC).

In some embodiments, the storage unit 215 stores files, such as drivers, libraries and saved programs. In some embodiments, the storage unit 215 stores user data, e.g., user preferences and user programs. In some embodiments, the computer system 201 in some cases includes one or more additional data storage units that are located external to the computer system 201 (e.g., on a remote server that is in communication with the computer system 201 through an intranet or the Internet).

In some embodiments, the computer system 201 communicates with one or more remote computer systems through the network 230. In some embodiments, the computer system 201 communicates with a remote computer system of a user (e.g., a subject, an end user, a consumer, a healthcare provider, an imaging technician, etc.). Examples of remote computer systems include personal computers (e.g., portable PC), slate or tablet PC's (e.g., Apple® iPad, Samsung® Gala2 Tab), telephones, Smart phones (e.g., Apple® iphone, Android-enabled device, Blackberry®), or personal digital assistants. In some embodiments, the user accesses the computer system 201 via the network 230.

In some embodiments, methods as described herein are implemented by way of machine (e.g., computer processor) executable code stored on an electronic storage location of the computer system 201, such as, for example, on the memory 210 or electronic storage unit 215. In some embodiments, the machine executable or machine readable code is be provided in the form of software. In some embodiments, during use, the code is executed by the processor 205. In some cases, the code is retrieved from the storage unit 215 and stored on the memory 210 for ready access by the processor 205. In some situations, the electronic storage unit 215 is precluded, and machine-executable instructions are stored on memory 210.

In some embodiments, the code is pre-compiled and configured for use with a machine having a processor adapted to execute the code, or is compiled during runtime. In some embodiments, the code is supplied in a programming language that is selected to enable the code to execute in a pre-compiled or as-compiled fashion.

In some embodiments, aspects of the systems and methods provided herein, such as the computer system 201, is embodied in programming. Various aspects of the technology are thought of as “products” or “articles of manufacture” typically in the form of machine (or processor) executable code and/or associated data that is carried on or embodied in a type of machine readable medium. In some embodiments, machine-executable code is stored on an electronic storage unit, such as memory (e.g., read-only memory, random-access memory, flash memory) or a hard disk. In some embodiments, “storage” type media includes any or all of the tangible memory of the computers, processors or the like, or associated modules thereof, such as various semiconductor memories, tape drives, disk drives and the like. In some embodiments, the “storage” type media provides non-transitory storage at any time for the software programming. In some embodiments, all or portions of the software are at times communicated through the Internet or various other telecommunication networks. In some embodiments, such communications, for example, enable loading of the software from one computer or processor into another, for example, from a management server or host computer into the computer platform of an application server. In some embodiments, thus, another type of media that bears the software elements includes optical, electrical and electromagnetic waves, such as used across physical interfaces between local devices, through wired and optical landline networks and over various air-links. In some embodiments, the physical elements that carry such waves, such as wired or wireless links, optical links or the like, also are considered as media bearing the software. In some embodiments, as used herein, unless restricted to non-transitory, tangible “storage” media, terms such as computer or machine “readable medium” refer to any medium that participates in providing instructions to a processor for execution.

In some embodiments, hence, a machine readable medium, such as computer-executable code, takes many forms, including but not limited to, a tangible storage medium, a carrier wave medium or physical transmission medium. In some embodiments, non-volatile storage media including, for example, optical or magnetic disks, or any storage devices in any computer(s) or the like, is used to implement the databases, etc. shown in the drawings. In some embodiments, volatile storage media include dynamic memory, such as main memory of such a computer platform. In some embodiments, tangible transmission media include coaxial cables; copper wire and fiber optics, including the wires that comprise a bus within a computer system. In some embodiments, carrier-wave transmission media takes the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer reads programming code and/or data. In some embodiments, many of these forms of computer readable media are involved in carrying one or more sequences of one or more instructions to a processor for execution.

In some embodiments, the computer system 201 includes or is in communication with an electronic display 235 that comprises a user interface (UI) 240 for providing, for example, a portal for a user to view a plurality of sequences or select a guide RNA candidate. In some embodiments, the portal is provided through an application programming interface (API). In some embodiments, a user or entity also interacts with various elements in the portal via the UI. Examples of UI's include, without limitation, a graphical user interface (GUI) and web-based user interface.

In some embodiments, methods and systems of the present disclosure are implemented by way of one or more algorithms. In some embodiments, an algorithm is implemented by way of software upon execution by the central processing unit 205. In some embodiments, for example, the algorithm is configured to identify one or more PAMS in the target sequence (e.g., consensus sequence), identify one or more guide target sequences in proximity to the one or more PAMs, and/or calculate one or more criteria based on the one or more guide target sequences or a guide RNA corresponding to at least one of the one more guide target sequence. In some embodiments, an algorithm is also configured align a plurality of sequences, as described herein.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

### NUMBERED EMBODIMENTS

1. A composition comprising:
   (a) a Clustered Regularly Interspaced Short Palindromic Repeat (CRISPR)-associated endonuclease or a nucleic acid sequence encoding the CRISPR-associated endonuclease; and
   (b) one or more guide RNAs (gRNAs) or a nucleic acid sequence encoding the one or more gRNAs, the one or more gRNA hybridizes or is complementary to a target nucleic acid sequence within a Hepatitis B Virus (HBV) genome, the HBV genome comprising at least about 90% sequence identity to any one of SEQ ID NOs: 2-5.
2. The composition of embodiment 1, wherein the HBV genome comprises a sequence of any one of SEQ ID NOs: 2-5.
3. The composition of embodiment 1 or 2, wherein a gRNA of the one or more gRNAs is encoded by a sequence according to any one of SEQ ID NOs: 6-272 or a sequence according to any one of SEQ ID NOs: 6-272 comprising 1, 2, or 3 modifications.
4. The composition of embodiment 3, wherein the modification is a substitution, deletion, insertion, or a combination thereof.
5. The composition of any one of embodiments 1-4, wherein a gRNA comprises a region that hybridizes to a target nucleic acid sequence within the HBV genome, the HBV comprising at least about 90% sequence identity to any one of SEQ ID NOs: 2-5.
6. The composition of any one of embodiments 1-5, wherein the target nucleic acid sequence is located within a structural gene, non-structural gene, or combinations thereof.
7. The composition of any one of embodiments 1-5, wherein the target nucleic acid sequence is located within a C, X, P, or S region.
8. The composition of any one of embodiments 1-7, wherein the CRISPR-associated endonuclease is Type I, Type II, or Type III Cas endonuclease.
9. The composition of any one of embodiments 1-7, wherein the CRISPR-associated endonuclease is a Cas9 endonuclease, a Cas12 endonuclease, a CasX endonuclease, or a CasΦ endonuclease.
10. The composition of any one of embodiments 1-7, wherein the CRISPR-associated endonuclease is a Cas9 endonuclease.
11. The composition of embodiment 10, wherein the Cas9 endonuclease is a Staphylococcus aureus Cas9 endonuclease.
12. The composition of any one of embodiments 1-11, wherein the HBV is HBV-A genotype.
13. The composition of any one of embodiments 1-11, wherein the HBV is HBV-B genotype.
14. The composition of any one of embodiments 1-11, wherein the HBV is HBV-C genotype.
15. The composition of any one of embodiments 1-11, wherein the HBV is HBV-A, HBV-B, HBV-C, HBV-D, HBV-E, HBV-F, HBV-G, or HBV-H genotype.
16. The composition of any one of embodiments 1-11, wherein the HBV is HBV-A1, HBV-A2, HBV-QS-A3, or HBV-A4 genotype.
17. The composition of any one of embodiments 1-11, wherein the HBV is HBV-B1, HBV-B2, HBV-QS-B3, HBV-B4, or HBV-B5 genotype.
18. The composition of any one of embodiments 1-11, wherein the HBV is HBV-C1, HBV-QS-C2, HBV-C3, HBV-C4, HBV-C5, or HBV-C6-C15 genotype.
19. The composition of any one of embodiments 1-11, wherein the HBV is HBV-D1, HBV-D2, HBV-D3, HBV-D4, HBV-D5, or HBV-D6 genotype.
20. The composition of any one of embodiments 1-11, wherein the HBV is HBV-F1, HBV-F2, HBV-F3, or HBV-F4 genotype.
21. A CRISPR-Cas system comprising:
   (a) a Clustered Regularly Interspaced Short Palindromic Repeat (CRISPR)-associated endonuclease; and
   (b) one or more guide RNAs (gRNAs) or a nucleic acid sequence encoding the one or more gRNAs, the one or more gRNA hybridizes or is complementary to a target nucleic acid sequence within a Hepatitis B Virus (HBV) genome, the HBV genome comprising at least about 90% sequence identity to any one of SEQ ID NOs: 2-5.
22. A nucleic acid encoding the CRISPR-Cas system of embodiment 21.
23. A vector comprising a nucleic acid encoding:
   (a) a Clustered Regularly Interspaced Short Palindromic Repeat (CRISPR)-associated endonuclease; and
   (b) one or more guide RNAs (gRNAs) or a nucleic acid sequence encoding the one or more gRNAs, the one or more gRNA hybridizes or is complementary to a target nucleic acid sequence within a Hepatitis B Virus (HBV) genome, the HBV genome comprising at least about 90% sequence identity to any one of SEQ ID NOs: 2-5.
24. The vector of embodiment 23, wherein the HBV genome comprises a sequence of any one of SEQ ID NOs: 2-5.
25. The vector of any one of the preceding embodiments, wherein a gRNA of the one or more gRNAs is encoded by a sequence according to any one of SEQ ID NOs: 6-272 or a sequence according to any one of SEQ ID NOs: 6-272 comprising 1, 2, or 3 modifications.
26. The vector of embodiment 25, wherein the modification is a substitution, deletion, insertion, or a combination thereof.
27. The vector of any one of the preceding embodiments, wherein a gRNA comprises a region that hybridizes to a target nucleic acid sequence within the HBV genome, the HBV genome comprising at least about 90% sequence identity to any one of SEQ ID NOs: 2-5.
28. The vector of any one of the preceding embodiments, wherein the target nucleic acid sequence is located within a structural gene, non-structural gene, or combinations thereof.
29. The vector of any one of the preceding embodiments, wherein n the target nucleic acid sequence is located within a C, X, P, or S region.
30. The vector of any one of the preceding embodiments, wherein the CRISPR-associated endonuclease is Type I, Type II, or Type III Cas endonuclease.
31. The vector of any one of the preceding embodiments, wherein the CRISPR-associated endonuclease is a Cas9 endonuclease, a Cas12 endonuclease, a CasX endonuclease, or a CasΦ endonuclease.
32. The vector of any one of the preceding embodiments, wherein the CRISPR-associated endonuclease is a Cas9 endonuclease.
33. The vector of embodiment 32, wherein the Cas9 endonuclease is a Staphylococcus aureus Cas9 endonuclease.
34. The vector of any one of the preceding embodiments, wherein, wherein the HBV is HBV-A genotype.
35. The vector of any one of the preceding embodiments, wherein the HBV is HBV-B genotype.
36. The vector of any one of the preceding embodiments, wherein the HBV is HBV-C genotype.
37. The vector of any one of the preceding embodiments, wherein the HBV is HBV-A, HBV-B, HBV-C, HBV-D, HBV-E, HBV-F, HBV-G, or HBV-H genotype.
38. The vector of any one of the preceding embodiments, wherein the HBV is HBV-A1, HBV-A2, HBV-QS-A3, or HBV-A4 genotype.
39. The vector of any one of the preceding embodiments, wherein the HBV is HBV-B1, HBV-B2, HBV-QS-B3, HBV-B4, or HBV-B5 genotype.
40. The vector of any one of the preceding embodiments, wherein the HBV is HBV-C1, HBV-QS-C2, HBV-C3, HBV-C4, HBV-C5, or HBV-C6-C15 genotype.
41. The vector of any one of the preceding embodiments, wherein the HBV is HBV-D1, HBV-D2, HBV-D3, HBV-D4, HBV-D5, or HBV-D6 genotype.
42. The vector of any one of the preceding embodiments, wherein the HBV is HBV-F1, HBV-F2, HBV-F3, or HBV-F4 genotype.
43. The vector of any one of the preceding embodiments, wherein the nucleic acid further comprises a promoter.
44. The vector of embodiment 43, wherein the promoter is a ubiquitous promoter.
45. The vector of embodiment 43, wherein the promoter is a tissue-specific promoter.
46. The vector of embodiment 43, wherein the promoter is a constitutive promoter.
47. The vector of embodiment 43, wherein the promoter is a human cytomegalovirus promoter.
48. The vector of any one of the preceding embodiments, wherein the nucleic acid further comprises an enhancer element.
49. The vector of embodiment 48, wherein the enhancer element is a human cytomegalovirus enhancer element.
50. The vector of any one of the preceding embodiments, wherein the nucleic acid further comprises a 5′ ITR element and 3′ ITR element.
51. The vector of any one of the preceding embodiments, wherein the vector is an adeno-associated virus (AAV) vector.
52. The vector of embodiment 51, wherein the adeno-associated virus (AAV) vector is an AAV2, AAV5, AAV6, AAV7, AAV8, or AAV9 vector.
53. The vector of embodiment 51, wherein the vector is an AAV6 vector or an AAV9 vector.
54. A method of excising part or all of a Hepatitis B Virus (HBV) sequence from a cell, the method comprising providing to the cell the composition of any one of embodiments 1-14, the CRISPR-Cas system of embodiment 21, or the vector of any one of the preceding embodiments.
55. A method of inhibiting or reducing Hepatitis B Virus (HBV) replication in a cell, the method comprising providing to the cell the composition of any one of embodiments 1-14, the CRISPR-Cas system of embodiment 21, or the vector of any one of the preceding embodiments.
56. The method of embodiment 54 or 55, wherein the cell is in a subject.
57. The method of embodiment 57, wherein the subject is a human.
58. A composition comprising:
   (a) a Clustered Regularly Interspaced Short Palindromic Repeat (CRISPR)-associated endonuclease or a nucleic acid sequence encoding the CRISPR-associated endonuclease; and
   (b) one or more guide RNAs (gRNAs) or a nucleic acid sequence encoding the one or more gRNAs, the one or more gRNA hybridizes or is complementary to a target nucleic acid sequence within a Hepatitis B Virus (HBV) genome, the HBV genome comprising at least about 90% sequence identity to any one of SEQ ID NOs: 2-5.
59. The composition of embodiment 58, wherein the HBV genome comprises a sequence of any one of SEQ ID NOs: 2-5.
60. The composition of embodiment 58 or 59, wherein a gRNA of the one or more gRNAs is encoded by a sequence according to any one of SEQ ID NOs: 272-548 sequence according to any one of SEQ ID NOs: 272-548 comprising 1, 2, or 3 modifications.
61. The composition of embodiment 60, wherein the modification is a substitution, deletion, insertion, or a combination thereof.
62. The composition of any one of embodiments 58-61, wherein a gRNA comprises a region that hybridizes to a target nucleic acid sequence within the HBV genome, the HBV comprising at least about 90% sequence identity to any one of SEQ ID NOs: 2-5.
63. The composition of any one of embodiments 58-62, wherein the target nucleic acid sequence is located within a structural gene, non-structural gene, or combinations thereof.
64. The composition of any one of embodiments 58-62, wherein the target nucleic acid sequence is located within a C, X, P, or S region.
65. The composition of any one of embodiments 58-64, wherein the CRISPR-associated endonuclease is Type I, Type II, or Type III Cas endonuclease.
66. The composition of any one of embodiments 58-64, wherein the CRISPR-associated endonuclease is a CasX endonuclease, a Cas12 endonuclease, a CasX endonuclease, or a CasΦ endonuclease.
67. The composition of any one of embodiments 58-64, wherein the CRISPR-associated endonuclease is a CasX endonuclease.
68. The composition of embodiment 67, wherein the CasX endonuclease is a deltaproteobacteria CasX or planctomycetes CasX endonuclease.
69. The composition of embodiment 68, the CasX endonuclease is a deltaproteobacteria CasX endonuclease.
70. The composition of embodiment 68, the CasX endonuclease is a planctomycetes CasX endonuclease.
71. The composition of any one of embodiments 58-70, wherein the HBV is HBV-A genotype.
72. The composition of any one of embodiments 58-70, wherein the HBV is HBV-B genotype.
73. The composition of any one of embodiments 58-70, wherein the HBV is HBV-C genotype.
74. The composition of any one of embodiments 58-70, wherein the HBV is HBV-A, HBV-B, HBV-C, HBV-D, HBV-E, HBV-F, HBV-G, or HBV-H genotype.
75. The composition of any one of embodiments 58-70, wherein the HBV is HBV-A1, HBV-A2, HBV-QS-A3, or HBV-A4 genotype.
76. The composition of any one of embodiments 58-70, wherein the HBV is HBV-B1, HBV-B2, HBV-QS-B3, HBV-B4, or HBV-B5 genotype.
77. The composition of any one of embodiments 58-70, wherein the HBV is HBV-C1, HBV-QS-C2, HBV-C3, HBV-C4, HBV-C5, or HBV-C6-C15 genotype.
78. The composition of any one of embodiments 58-70, wherein the HBV is HBV-D1, HBV-D2, HBV-D3, HBV-D4, HBV-D5, or HBV-D6 genotype.
79. The composition of any one of embodiments 58-70, wherein the HBV is HBV-F1, HBV-F2, HBV-F3, or HBV-F4 genotype.
80. A CRISPR-Cas system comprising:
   (a) a Clustered Regularly Interspaced Short Palindromic Repeat (CRISPR)-associated endonuclease; and
   (b) one or more guide RNAs (gRNAs) or a nucleic acid sequence encoding the one or more gRNAs, the one or more gRNA hybridizes or is complementary to a target nucleic acid sequence within a Hepatitis B Virus (HBV) genome, the HBV genome comprising at least about 90% sequence identity to any one of SEQ ID NOs: 2-5.
81. A nucleic acid encoding the CRISPR-Cas system of embodiment 21.
82. A vector comprising a nucleic acid encoding:
   (a) a Clustered Regularly Interspaced Short Palindromic Repeat (CRISPR)-associated endonuclease; and
   (b) one or more guide RNAs (gRNAs) or a nucleic acid sequence encoding the one or more gRNAs, the one or more gRNA hybridizes or is complementary to a target nucleic acid sequence within a Hepatitis B Virus (HBV) genome, the HBV genome comprising at least about 90% sequence identity to any one of SEQ ID NOs: 2-5.
83. The vector of embodiment 82, wherein the HBV genome comprises a sequence of any one of SEQ ID NOs: 2-5.
84. The vector of any one of the preceding embodiments, wherein a gRNA of the one or more gRNAs is encoded by a sequence according to any one of SEQ ID NOs: 272-548 or a sequence according to any one of SEQ ID NOs: 272-548 comprising 1, 2, or 3 modifications.
85. The vector of embodiment 84, wherein the modification is a substitution, deletion, insertion, or a combination thereof.
86. The vector of any one of the preceding embodiments, wherein a gRNA comprises a region that hybridizes to a target nucleic acid sequence within the HBV genome, the HBV genome comprising at least about 90% sequence identity to any one of SEQ ID NOs: 2-5.
87. The vector of any one of the preceding embodiments, wherein the target nucleic acid sequence is located within a structural gene, non-structural gene, or combinations thereof.
88. The vector of any one of the preceding embodiments, wherein the target nucleic acid sequence is located within a C, X, P, or S region.
89. The vector of any one of the preceding embodiments, wherein the CRISPR-associated endonuclease is Type I, Type II, or Type III Cas endonuclease.
90. The vector of any one of the preceding embodiments, wherein the CRISPR-associated endonuclease is a Cas9 endonuclease, a Cas12 endonuclease, a CasX endonuclease, or a CasΦ endonuclease.
91. The vector of any one of the preceding embodiments, wherein the CRISPR-associated endonuclease is a Cas9 endonuclease.
92. The vector of embodiment 91, wherein the Cas9 endonuclease is a Staphylococcus aureus Cas9 endonuclease.
93. The vector of any one of the preceding embodiments, wherein the HBV is HBV-A genotype.
94. The vector of any one of the preceding embodiments, wherein the HBV is HBV-B genotype.
95. The vector of any one of the preceding embodiments, wherein the HBV is HBV-C genotype.
96. The vector of any one of the preceding embodiments, wherein the HBV is HBV-A, HBV-B, HBV-C, HBV-D, HBV-E, HBV-F, HBV-G, or HBV-H genotype.
97. The vector of any one of the preceding embodiments, wherein the HBV is HBV-A1, HBV-A2, HBV-QS-A3, or HBV-A4 genotype.
98. The vector of any one of the preceding embodiments, wherein the HBV is HBV-B1, HBV-B2, HBV-QS-B3, HBV-B4, or HBV-B5 genotype.
99. The vector of any one of the preceding embodiments, wherein the HBV is HBV-C1, HBV-QS-C2, HBV-C3, HBV-C4, HBV-C5, or HBV-C6-C15 genotype.
100. The vector of any one of the preceding embodiments, wherein the HBV is HBV-D1, HBV-D2, HBV-D3, HBV-D4, HBV-D5, or HBV-D6 genotype.
101. The vector of any one of the preceding embodiments, wherein the HBV is HBV-F1, HBV-F2, HBV-F3, or HBV-F4 genotype.
102. The vector of any one of the preceding embodiments, wherein the nucleic acid further comprises a promoter.
103. The vector of embodiment 102, wherein the promoter is a ubiquitous promoter.
104. The vector of embodiment 102, wherein the promoter is a tissue-specific promoter.
105. The vector of embodiment 102, wherein the promoter is a constitutive promoter.
106. The vector of embodiment 102, wherein the promoter is a human cytomegalovirus promoter.
107. The vector of any one of the preceding embodiments, wherein the nucleic acid further comprises an enhancer element.
108. The vector of embodiment 107, wherein the enhancer element is a human cytomegalovirus enhancer element.
109. The vector of any one of the preceding embodiments, wherein the nucleic acid further comprises a 5′ ITR element and 3′ ITR element.
110. The vector of any one of the preceding embodiments, wherein the vector is an adeno-associated virus (AAV) vector.
111. The vector of embodiment 110, wherein the adeno-associated virus (AAV) vector is an AAV2, AAV5, AAV6, AAV7, AAV8, or AAV9 vector.
112. The vector of embodiment 110, wherein the vector is an AAV6 vector or an AAV9 vector.
113. A method of excising part or all of a Hepatitis B Virus (HBV) sequence from a cell, the method comprising providing to the cell the composition of any one of embodiments 58-79, the CRISPR-Cas system of embodiment 21, or the vector of any one of the preceding embodiments.
114. A method of inhibiting or reducing Hepatitis B Virus (HBV) replication in a cell, the method comprising providing to the cell the composition of any one of embodiments 58-79, the CRISPR-Cas system of embodiment 21, or the vector of any one of the preceding embodiments.
115. The method of embodiment 113 or 114, wherein the cell is in a subject.
116. The method of embodiment 115, wherein the subject is a human.
117. A computer-implemented system to select, evaluate, or prioritize a target site and/or a guide RNA candidate, the system comprising: at least one processor, a memory, and instructions executable by the at least one processor comprising:
   a. a data storage system comprising a target sequence (e.g., a consensus sequence); and
   b. one or more modules communicatively coupled to the data storage system, wherein the one or more modules are configured to:
      i. identify one or more targeting sites in the target sequence;
      ii. identify one or more guide target sequences in proximity to the one or more targeting sites; and
      iii. calculate one or more criteria based on the one or more guide target sequences or a guide RNA corresponding to at least one of the one more guide target sequence,
   wherein the guide RNA candidate is selected based on the one or more criteria of step (b) (iii).
118. A computer-implemented system to select, evaluate, or prioritize a target site and/or a guide RNA candidate, the system comprising: at least one processor, a memory, and instructions executable by the at least one processor comprising:
   a. a data storage system comprising a target sequence (e.g., consensus sequence);
   b. a targeting site identifier configured to identify one or more targeting sites in the target sequence;
   c. a proximity identifier configured to identify one or more guide target sequences in proximity to the one or more targeting sites; and
   d. a criteria analysis module configured to calculate one or more criteria based on the one or more guide target sequences or a guide RNA corresponding to at least one of the one or more guide target sequence, wherein the guide RNA candidate is selected based on the one or more criteria of step (d).
119. The computer-implemented system of embodiment 117 or 118, wherein the one or more targeting sites are adjacent or proximal to (e.g., within 10 bp) protospacer adjacent motifs (PAMs).
120. The computer-implemented system of embodiment 117 or 118, wherein the target sequence is a reference sequence.
121. The computer-implemented system of embodiment 117 or 118, wherein the target sequence is a single natural or computed sequence.
122. The computer-implemented system of embodiment 117 or 118, wherein the target sequence is a single natural sequence.
123. The computer-implemented system of embodiment 117 or 118, wherein the target sequence is a single computed sequence.
124. The computer-implemented system of embodiment 117 or 118, wherein the target sequence is generated by aligning a plurality of sequences.
125. The computer-implemented system of embodiment 124, wherein the target sequence is a consensus sequence generated by a module configured to (1) align the plurality of sequences.
126. The computer-implemented system of embodiment 125, wherein the consensus is generated by a module configured to (1) align the plurality of sequences with common geographies, pathologies, or combinations thereof.
127. The computer-implemented system of embodiment 126, wherein the module is further configured to (2) compare nucleotides at each position of the plurality of sequences; and (3) select a nucleotide at each position that appears at a frequency higher than other nucleotides.
128. The computer-implemented system of any one of embodiments 124-127, wherein the plurality of sequences comprises at least 5 sequences.
129. The computer-implemented system of any one of embodiments 124-127, wherein the plurality of sequences comprises at least 50 sequences.
130. The computer-implemented system of any one of embodiments 124-127, wherein the plurality of sequences comprises at least 100 sequences.
131. The computer-implemented system of any one of embodiments 124-127, wherein the plurality of sequences comprises at least 500 sequences.
132. The computer-implemented system of any one of embodiments 124-127, wherein the plurality of sequences comprises at least 1000 sequences.
133. The computer-implemented system of embodiment 124, wherein the guide RNA candidate is compared against at least one (e.g., a plurality) of the plurality of sequences.
134. The computer-implemented system of embodiment 124, wherein the guide RNA candidate is compared against a sequence of a viral strain different from the plurality of sequences.
135. The computer-implemented system of embodiment 134, the sequence of a viral strain different from the plurality of sequences is a viral sequence reference.
136. The computer-implemented system of embodiment 134, the sequence of a viral strain different from the plurality of sequences is derived from multiple genome sequences (e.g., multiple genome sequences from a library of viral genome, e.g., Virus Pathogen Database and Analysis Resource (ViPR) and/or LANL Research Library Database).
137. The computer-implemented system of embodiment 124, wherein the guide RNA candidate is compared against a sequence of a human genome different from the plurality of sequences.
138. The computer-implemented system of embodiment 137, wherein the sequence of a human genome is a human reference sequence.
139. The computer-implemented system of embodiment 137, wherein the sequence of a human genome is derived from multiple genome sequences (e.g., multiple genome sequences from a library of human genome, e.g., from The 100,000 Genomes Project).
140. The computer-implemented system of embodiment 137, wherein the sequence of a human genome comprises one or more single nucleotide polymorphisms (SNPs).
141. The computer-implemented system of any one of embodiments 124-140, wherein the plurality of sequences are found in the data storage system.
142. The computer-implemented system of any one of embodiments 124-141, wherein the plurality of sequences are a plurality of viral sequences.
143. The computer-implemented system of embodiment 142, wherein the plurality of viral sequences are sequences from a single virus.
144. The computer-implemented system of embodiment 142, wherein the plurality of viral sequences are sequences from different viruses.
145. The computer-implemented system of embodiment 142, wherein the plurality of viral sequences are sequences within a same genotype of a virus.
146. The computer-implemented system of embodiment 142, wherein the plurality of viral sequences are sequences within different genotypes of a virus.
147. The computer-implemented system of embodiment 142, wherein the plurality of viral sequences are sequences within different subclades of a virus.
148. The computer-implemented system of embodiment 142, wherein the plurality of viral sequences are sequences within a same subclade of a virus.
149. The computer-implemented system of any one of embodiments 143-148, wherein the virus is Hepatitis B virus (HBV), Human Immunodeficiency virus (HIV), JC virus (JCV), herpes simplex virus (HSV), or SARS-CoV-2.
150. The computer-implemented system of embodiment 149, wherein the virus is Hepatitis B virus (HBV) and the genotype is HBV-A.
151. The computer-implemented system of embodiment 149, wherein the virus is Hepatitis B virus (HBV) and the genotype is HBV-B.
152. The computer-implemented system of embodiment 149, wherein the virus is Hepatitis B virus (HBV) and the genotype is HBV-C.
153. The computer-implemented system of embodiment 149, wherein the virus is Hepatitis B virus (HBV) and the different genotypes comprise HBV-A, HBV-B, HBV-C, or combinations thereof.
154. The computer-implemented system of embodiment 149, wherein the virus is Hepatitis B virus (HBV) and different genotypes comprise HBV-A, HBV-B, HBV-C, HBV-D, HBV-E, HBV-F, HBV-G, HBV-H, or combinations thereof.
155. The computer-implemented system of embodiment 149, wherein the virus is Hepatitis B virus (HBV) and various subclades within HBV-A, HBV-B, HBV-C, HBV-D, HBV-E, HBV-F, HBV-G, HBV-H, or combinations thereof.
156. The computer-implemented system of embodiment 155, wherein the subclade within HBV-A comprises HBV-A1, HBV-A2, HBV-QS-A3, HBV-A4, or combinations thereof.
157. The computer-implemented system of embodiment 155, wherein the subclade within HBV-B comprises HBV-B1, HBV-B2, HBV-QS-B3, HBV-B4, HBV-B5, or combinations thereof.
158. The computer-implemented system of embodiment 155, wherein the subclade within HBV-C comprises HBV-C1, HBV-QS-C2, HBV-C3, HBV-C4, HBV-C5, HBV-C6-C15, or combinations thereof.
159. The computer-implemented system of embodiment 155, wherein the subclade within HBV-D comprises HBV-D1, HBV-D2, HBV-D3, HBV-D4, HBV-D5, HBV-D6, or combinations thereof.
160. The computer-implemented system of embodiment 155, wherein the subclade within HBV-F comprises HBV-F1, HBV-F2, HBV-F3, HBV-F4, or combinations thereof.
161. The computer-implemented system of any one of embodiments 117-160, wherein the one or more criteria are user-supplied criteria.
162. The computer-implemented system of any one of embodiments 117-160, wherein the one or more criteria are based on positional entropy, conservation, a knockout score, an overlapping reading frame, a gene location, a coding region, a non-coding region, predicted cutting rate or efficiency, efficacy, a frequency of a targeting site (e.g., a PAM), or combinations thereof.
163. The computer-implemented system of embodiment 161, wherein the knockout score is an on-target knockout score.
164. The computer-implemented system of embodiment 163, wherein the on-target knockout score is determined by efficiency of cleavage.
165. The computer-implemented system of embodiment 163, wherein the on-target knockout score is determined using an algorithm (e.g., Azimuth 2.0).
166. The computer-implemented system of embodiment 161, wherein the knockout score is an off-target knockout score.
167. The computer-implemented system of embodiment 166, wherein the off-target knockout score is determined using an algorithm (e.g., COSMID, python script, Cas-OFFinder).
168. The computer-implemented system of any one of embodiments 117-167, wherein the guide RNA candidate is selected by a user.
169. The computer-implemented system of any one of embodiments 117-167, wherein the guide RNA candidate is selected by a module configured to analyze the one or more criteria and select the guide RNA candidate based on the one or more criteria.
170. The computer-implemented system of any one of embodiments 117-169, wherein the guide RNA candidate is a clinical candidate.
171. The computer-implemented system of any one of embodiments 117-170, wherein the guide RNA candidate is encoded by a sequence according to any one of SEQ ID NOs: 6-549.
172. The computer-implemented system of any one of embodiments 117-170, wherein the target sequence comprises at least about 90% sequence identity to any one of SEQ ID NOs: 2-5.
173. The computer-implemented system of embodiment 172, wherein the target sequence comprises at least 15 nucleotides.
174. The computer-implemented system of embodiment 172, wherein the target sequence comprises at least 20 nucleotides.
175. The computer-implemented system of embodiment 172, wherein the target sequence comprises at least 30 nucleotides.
176. The computer-implemented system of embodiment 172, wherein the target sequence comprises about 20 nucleotides.
177. A method of selecting, evaluating, or prioritizing a guide RNA candidate, comprising:
   a. providing a target sequence (e.g., consensus sequence);
   b. identifying one or more targeting sites in the target sequence;
   c. identifying one or more guide target sequences in proximity to the one or more targeting sites;
   d. calculating one or more criteria based on the one more guide target sequences or a guide RNA corresponding to at least one of the one more guide target sequence; and
   e. selecting the guide RNA candidate based on the one or more criteria of step (d).
178. The method of embodiment 177, wherein the one or more targeting sites are protospacer adjacent motifs (PAMs).
179. The method of embodiment 177, wherein the target sequence is a reference sequence.
180. The method of embodiment 177, wherein the target sequence is a single natural or computed sequence.
181. The method of embodiment 177, wherein the target sequence is a single natural sequence.
182. The method of embodiment 177, wherein the target sequence is a single computed sequence.
183. The method of embodiment 177, wherein the target sequence is generated by aligning a plurality of sequences.
184. The method of embodiment 183, wherein the target sequence is a consensus sequence is determined by aligning the plurality of sequences.
185. The method of embodiment 184, wherein the consensus is determined by aligning sequences with common geographies, pathologies, or combinations thereof.
186. The method of embodiment 185, wherein the consensus sequence is determined by comparing nucleotides at each position of the plurality of sequences; and selecting a nucleotide at each position that appears at a frequency higher than other nucleotides.
187. The method of any one of embodiments 183-186, wherein the plurality of sequences comprises at least 5 sequences.
188. The method of any one of embodiments 183-186, wherein the plurality of sequences comprises at least 50 sequences.
189. The method of any one of embodiments 183-186, wherein the plurality of sequences comprises at least 100 sequences.
190. The method of any one of embodiments 183-186, wherein the plurality of sequences comprises at least 500 sequences.
191. The method of any one of embodiments 183-186, wherein the plurality of sequences comprises at least 1000 sequences.
192. The method of any one of embodiments 183-191, wherein the guide RNA candidate is compared against at least one (e.g., a plurality) of the plurality of sequences.
193. The method of any one of embodiments 183-191, wherein the guide RNA candidate is compared against a sequence of a viral strain different from the plurality of sequences.
194. The method of any one of embodiments 183-191, the sequence of a viral strain different from the plurality of sequences is a viral sequence reference.
195. The method of embodiment 196, the sequence of a viral strain different from the plurality of sequences is derived from multiple genome sequences (e.g., multiple genome sequences from a library of viral genome, e.g., Virus Pathogen Database and Analysis Resource (ViPR) and/or LANL Research Library Database).
196. The method of any one of embodiments 183-191, wherein the guide RNA candidate is compared against a sequence of a human genome different from the plurality of sequences.
197. The method of embodiment 196, wherein the sequence of a human genome is a human reference sequence.
198. The method of embodiment 196, wherein the sequence of a human genome is derived from multiple genome sequences (e.g., multiple genome sequences from a library of human genome, e.g., from The 100,000 Genomes Project).
199. The method of embodiment 196, wherein the sequence of a human genome comprises one or more single nucleotide polymorphisms (SNPs).
200. The method of any one of embodiments 183-199, wherein the plurality of sequences are a plurality of viral sequences.
201. The method of embodiment 200, wherein the plurality of viral sequences are sequences from one virus.
202. The method of embodiment 200, wherein the plurality of viral sequences are sequences from different viruses.
203. The method of embodiment 200, wherein the plurality of viral sequences are sequences within a same genotype of a virus.
204. The method of embodiment 200, wherein the plurality of viral sequences are sequences within different genotypes of a virus.
205. The method of embodiment 200, wherein the plurality of viral sequences are sequences within different subclades of a virus.
206. The method of embodiment 200, wherein the plurality of viral sequences are sequences within a same subclade of a virus.
207. The method of any one of embodiments 200-206, wherein the virus is Hepatitis B virus (HBV), Human Immunodeficiency virus (HIV), JC virus (JCV), herpes simplex virus (HSV), or SARS-CoV-2.
208. The method of embodiment 207, wherein the virus is Hepatitis B virus (HBV) and a genotype is HBV-A.
209. The method of embodiment 207, wherein the virus is Hepatitis B virus (HBV) and a genotype is HBV-B.
210. The method of embodiment 207, wherein the virus is Hepatitis B virus (HBV) and genotype is HBV-C.
211. The method of embodiment 207, wherein the virus is Hepatitis B virus (HBV) and different genotypes comprise HBV-A, HBV-B, HBV-C, or combinations thereof.
212. The method of embodiment 207, wherein the virus is Hepatitis B virus (HBV) and different genotypes comprise HBV-A, HBV-B, HBV-C, HBV-D, HBV-E, HBV-F, HBV-G, HBV-H, or combinations thereof.
213. The method of embodiment 207, wherein the virus is Hepatitis B virus (HBV) and various subclades within HBV-A, HBV-B, HBV-C, HBV-D, HBV-E, HBV-F, HBV-G, HBV-H, or combinations thereof.
214. The method of embodiment 213, wherein the subclade within HBV-A comprises HBV-A1, HBV-A2, HBV-QS-A3, HBV-A4, or combinations thereof.
215. The method of embodiment 213, wherein the subclade within HBV-B comprises HBV-B1, HBV-B2, HBV-QS-B3, HBV-B4, HBV-B5, or combinations thereof.
216. The method of embodiment 213, wherein the subclade within HBV-C comprises HBV-C1, HBV-QS-C2, HBV-C3, HBV-C4, HBV-C5, HBV-C6-C15, or combinations thereof.
217. The method of embodiment 213, wherein the subclade within HBV-D comprises HBV-D1, HBV-D2, HBV-D3, HBV-D4, HBV-D5, HBV-D6, or combinations thereof.
218. The method of embodiment 213, wherein the subclade within HBV-F comprises HBV-F1, HBV-F2, HBV-F3, HBV-F4, or combinations thereof.
219. The method of any one of embodiments 177-211, wherein the one or more criteria are user-supplied criteria.
220. The method of any one of embodiments 177-211, wherein the one or more criteria are based on positional entropy, conservation, a knockout score, an overlapping reading frame, a gene location, a coding region, a non-coding region, predicted cutting rate or efficiency, efficacy, a frequency of a target site (e.g., a PAM), or combinations thereof.
221. The method of embodiment 220, wherein the knockout score is an on-target knockout score.
222. The method of embodiment 221, wherein the on-target knockout score is determined by efficiency of cleavage.
223. The method of embodiment 221, wherein the on-target knockout score is determined using an algorithm (e.g., Azimuth 2.0).
224. The method of embodiment 220, wherein the knockout score is an off-target knockout score.
225. The method of embodiment 224, wherein the off-target knockout score or off-target cutting potential is determined using an algorithm (e.g., COSMID, python script, Cas-OFFinder).
226. The method of any one of embodiments 177-225, wherein the guide RNA candidate is a clinical candidate.
227. The method of any one of embodiments 177-226, wherein the guide RNA candidate is encoded by a sequence according to any one of SEQ ID NOs: 6-549.
228. The method of any one of embodiments 177-226, wherein the target sequence comprises at least about 90% sequence identity to any one of SEQ ID NOs: 2-5.
229. The method of embodiment 228, wherein the target sequence comprises at least 15 nucleotides.
230. The method of embodiment 228, wherein the target sequence comprises at least 20 nucleotides.
231. The method of embodiment 228, wherein the target sequence comprises at least 30 nucleotides.
232. The method of embodiment 228, wherein the target sequence comprises about 20 nucleotides.
233. A computer-implemented system to select, evaluate, or prioritize a targeting site, the system comprising: at least one processor, a memory, and instructions executable by the at least one processor comprising:
   a. a data storage system comprising a target sequence (e.g., consensus sequence) comprising at least about 90% sequence identity to any one of SEQ ID NOs: 2-5;
   b. a criteria analysis module configured to calculate one or more criteria of the targeting site; and
   c. a target identifier for selecting the target site based on the one or more criteria of step (b).
234. The computer-implemented system of embodiment 233, wherein the one or more criteria is based on positional entropy, conservation, a knockout score, an overlapping reading frame, a gene location, a coding region, a non-coding region, predicted cutting rate or efficiency, efficacy, a frequency of a targeting site (e.g., a PAM), or combinations thereof.
235. A computer-implemented system to select, evaluate, or prioritize a targeting site, the system comprising: at least one processor, a memory, and instructions executable by the at least one processor comprising:
   a. a data storage system comprising a target sequence (e.g., consensus sequence; and
   b. a criteria analysis module configured to calculate one or more criteria of the targeting site, wherein the one or more criteria is based on positional entropy, conservation, a knockout score, an overlapping reading frame, a gene location, a coding region, a non-coding region, predicted cutting rate or efficiency, efficacy, a frequency of a targeting site (e.g., a PAM), or combinations thereof; and
   c. a target identifier for selecting the target site based on the one or more criteria of step (b).
236. The computer-implemented system of any one of embodiments 233-235, wherein the targeting site is recognized by a zinc finger nuclease, a transcription activator-like effector nuclease (TALEN), a meganuclease, or a CRISPR-associated protein (e.g., Cas).

1. A composition comprising:
(a) a Clustered Regularly Interspaced Short Palindromic Repeat (CRISPR)-associated endonuclease or a nucleic acid sequence encoding the CRISPR-associated endonuclease; and
(b) one or more guide RNAs (gRNAs) or a nucleic acid sequence encoding the one or more gRNAs, the one or more gRNA hybridizes or is complementary to a target nucleic acid sequence within a Hepatitis B Virus (HBV) genome, the HBV genome comprising at least about 90% sequence identity to any one of SEQ ID NOs: 2-5.

2.-22. (canceled)
